(19) United States
(12) Patent Application Publication
(10) Pub. No.: US 2002/0198254 A1
(43) Pub. Date: Dec. 26, 2002

(54) Polyamide analogs

(76) Inventors: Mark D. Velligan, Momtara, CA (US); Alexander Khorlin, Mountain View, CA (US); Natalia B. Dyatkina, Mountain View, CA (US); Dong-Fang Shi, San Mateo, CA (US); Janos Botyanszki, Cupertinto, CA (US); Sebastian Liehr, East Palo Alto, CA (US)

Correspondence Address: Gerald F. Swiss, Esq., BURNS, DOANE, SWECKER & MATHIS, L.L.P., P.O. Box 1404, Alexandria, VA, 22313-1404, US

(21) Appl. No.: 10/026,963

(22) Filed: Dec. 27, 2001

Related U.S. Application Data
(60) Provisional application No. 60/258,842, filed on Dec. 27, 2000.

(51) Int. Cl. A61K 31/4015; C 07D 4 3/02

(52) U.S. Cl. 514/422; 548/518

(57) ABSTRACT

This invention is directed to novel antibacterial/antifungal/antiparasitic agents of Formula (I):

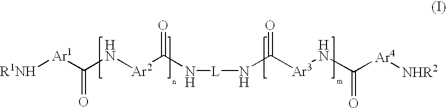

pharmaceutical compositions containing them, methods for their use and methods for preparing these compound

### CROSSREFERENCE TO RELATED APPLICATIONS

This application claims the benefit under 35 U.S.C. 119(e) of U.S. Provisional Application No. 60/258,842, filed on Dec. 27, 2000 (Attorney Docket 033052-003), the disclosure of which is incorporated herein in its entirety.

### BACKGROUND OF THE INVENTION

1. Field of Invention

The present invention provides novel polyamide compounds that are useful in the treatment of diseases caused by pathogenic organisms such as viruses, bacteria, parasites, and fungi. The compounds of the present invention are also useful in the treatment of cancer. Pharmaceutical compositions containing these compounds, methods of making and methods for using these compounds are also provided.

2. State of the Art

The binding of the antibacterial netropsin and distamycin to AT-rich sequences in the minor groove of double stranded DNA is a well studied phenomenon. Because such binding can be used to regulate DNA expression, e.g., by blocking and/or displacement of regulatory proteins, or by inhibiting the activity of enzymes acting on DNA, such as reverse transcriptase or topoisomerase, optimization of this binding has been the subject of numerous recent studies.

As described in a recent review by Bailly and Chaires (Bioconj. Chem. 9(5):513-38, 1998), the pyrrolecarboxamide unit in netropsin and distamycin is actually about 20% longer than required to perfectly match the corresponding base pair sequence in the minor groove. Accordingly, in oligomeric analogs having multiple binding moieties, successive binding moieties can become out of phase with the base pairs of the minor groove. Several studies have therefore been directed to dimers of netropsin or distamycin containing different linkers, in order to improve binding to longer target sequences. In these reports, effectiveness of various netropsin or distamycin dimers was determined, for example, in the inhibition of transcription by HIV-1 reverse transcriptase (M. Filipowsky et al., Biochemistry 35:15397-410, 1996), in the inhibition of mammalian DNA topoisomerase I (Z. Wang et al., Biochem. Pharmacol. 53:309-16, 1997), or in the inhibition of HIV 1 integrase (N. Neamati et al., Mol. Pharmacol. 54:280-90, 1998).

Preferred linkers in these studies included p-phenylene, trans-vinyl, cyclopropyl, 3,5-pyridyl, and six- and eight-carbon aliphatic chains. Several of these linkers restrict rotation around the linking group, thus reducing the extent of purely monodentate binding (e.g. by only one netropsin moiety; see Bailly, supra) which can occur with flexible linkers. However, Kissinger et al. (Chem. Res. Toxicol. 3(2):162-8, 1990) reported that aryl-linked groups had reduced DNA binding affinity compared to alkyl and alkylene linkers, and Neamati et al. (cited above) reported that the trans-vinyl linked compound was many times more potent (in inhibiting HIV-1 integrase) than the “more rigid” cyclobutanyl and norbornyl linkers. It was suggested in Wang and in Bailly (supra) that, for certain applications, the more rigid linkers (cyclopropyl and p-phenylene) may not allow for optimal simultaneous (bidentate) binding of the two netropsin moieties flanking the linker. Therefore, it would be desirable to provide compounds which reduce monodentate binding but which provide suitable geometries for bidentate binding and thus assist in combating diseases such as cancer and those caused by pathogenic agents such as bacteria and fungi.

The compounds of the present invention fulfill this need.

### SUMMARY OF THE INVENTION

In a first aspect, the present invention provides a polyamide compound of Formula (I):

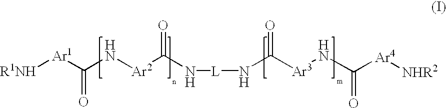

wherein:

R¹ and R² are, independently of each other:

(i) hydrogen;

(ii) alkyl; or

(iii) —COR³ wherein R³ is selected from the group consisting of alkyl, amino, monosubstituted amino, disubstituted amino, or alkyl substituted with one, two or three substituents selected from the group consisting of amino, monosubstituted amino, disubstituted amino, guanidino, amidino, aminoacyl, —NHCOR^{a} (wherein R^{a} is hydrogen, alkyl, substituted alkyl, aryl, substituted aryl, aralkyl, substituted aralkyl, cycloalkyl, substituted cycloalkyl, cycloalkylalkyl, substituted cycloalkylalkyl, heteroaryl, substituted heteroaryl, heteroaralkyl, substituted heteroaralkyl, or polyoxyalkylene), —NHCONHR^{a} (wherein R^{a} is as defined above), aryl, substituted aryl, heteroaryl, substituted heteroaryl, carboxy, alkoxycarbonyl, —OR^{b} (where R^{b} is hydrogen, alkyl, substituted alkyl, aryl, substituted aryl, aralkyl, substituted aralkyl, cycloalkyl, substituted cycloalkyl, cycloalkylalkyl, substituted cycloalkylalkyl, heteroaryl, substituted heteroaryl, heteroaralkyl, substituted heteroaralkyl, or polyoxyalkylene), and polyoxyalkylene, provided that at least one of R¹ and R² is a group that can form a pharmaceutically acceptable acid addition salt;

n and m are independently an integer from 0 to 4; and

Ar¹, Ar², Ar³, and Ar⁴ are independently selected from the group consisting of arylene, substituted arylene, and optionally substituted heteroarylene; and

L is:

(i) alkylene;

(ii) alkylene substituted with one, two or three substituent(s) selected from the group consisting of aryl, —CONHR⁴ (wherein R⁴ is hydrogen, alkyl, substituted alkyl, hydroxyalkyl, alkoxyalkyl, aminoalkyl, aryl, substituted aryl, aralkyl, substituted aralkyl, heteroaryl, substituted heteroaryl, heteroaralkyl, or substituted heteroaralkyl, heterocyclic, substituted heterocyclic, heterocyclicalkyl, heteroarylthioalkyl, or —(CHR⁵)ₙ₁—CO—(NH—Ar³—CO)ₘ—NH—Ar⁴—CO—NHR³ where n1 is 1 to 3, R⁵ is hydrogen or alkyl, substituted alkyl, and Ar³, m, Ar⁴, and R³ are as defined above), —CONHNHR⁶ [wherein R⁶ is alkyl, substituted alkyl, aryl, substituted aryl, aralkyl, substituted aralkyl, —COR⁷, —COOR⁸ (wherein R⁷ and R⁸ are independently of each other alkyl, substituted alkyl, aryl, substituted aryl, aralkyl, cycloalkyl, substituted cycloalkyl, cycloalkylalkyl, substituted cycloalkylalkyl, heteroaryl, substituted heteroaryl, or heteroaralkyl), heteroaryl, or heteroaralkyl], —NHR⁹ (wherein R⁹ is hydrogen, alkyl, substituted alkyl, hydroxyalkyl, alkoxyalkyl, aminoalkyl, aminoalkylcarbonyl, or heterocycliccarbonyl), and guanidino; or

(iii) -(alkylene)ₓ-Z-(alkylene)_{y}- wherein x, y and z are independently 0, 1, or 2 and Z and Z^{a} are, independently of each other, phenylene, cycloalkylene optionally fused to one or two phenylene ring(s), heterocyclene, —O—, —S—, —NR¹⁰— [wherein R¹⁰ is hydrogen, alkyl, substituted alkyl, cycloalkylcarbonyl, hydroxyalkyl, alkoxyalkyl, aminoalkyl, —CONHR⁴, —COR⁷, —COOR⁸ (where R⁴, R⁷ and R⁸ are as defined above), —SO₂R¹¹ (where R¹¹ is alkyl, substituted alkyl, aryl, substituted aryl, aralkyl, substituted aralkyl, heteroaryl, substituted heteroaryl, heteroaralkyl, or substituted heteroaralkyl) or —(CHR⁵)ₙ₂—NH—(CO—Ar³—NH)ₘ—CO—Ar⁴—NHR² where n2 is 2 to 4, R⁵ is hydrogen, alkyl, or substituted alkyl, and Ar³, m, Ar⁴, and R² are as defined above], —CO—NH—, or —NH—CO—, provided that when Z and/or Z^{a} is —NR¹⁰— then it is separated from another nitrogen atom by at least two carbon atoms;

or a pharmaceutically acceptable salt thereof.

In a second aspect, the present invention provides pharmaceutical compositions comprising a therapeutically effective amount of a compound of Formula (I) and a pharmaceutically suitable carrier.

In a third aspect, the present invention provides methods for the treatment of diseases caused by pathogenic organisms, which comprises administering to a mammal in need of such treatment a therapeutically effective amount of a compound of Formula (I) or a pharmaceutical composition containing a therapeutically effective amount of a compound of Formula (I).

In a fourth aspect, the present invention provides methods for the treatment of cancer, which comprises administering to a mammal in need of such treatment a therapeutically effective amount of a compound of Formula (I) or a pharmaceutical composition containing a therapeutically effective amount of a compound of Formula (I).

In a fifth aspect, the present invention provides the use of a compound of Formula (I) in the preparation of a medicament. Preferably, the medicament is for the treatment of diseases caused by pathogenic organisms or cancer.

### BRIEF DESCRIPTION OF THE DRAWINGS

FIGS. 1-14 illustrate various methods of preparing compounds of Formula (I).

FIGS. 15-16 illustrate some specific of compounds of Formula (I).

### DETAILED DESCRIPTION OF THE INVENTION

Unless otherwise stated, the following terms used in the specification and claims have the meanings given below:

“Alkyl” means a linear or branched saturated monovalent hydrocarbon radical of one to twenty, preferably one to ten, more preferably one to six carbon atoms, e.g., methyl, ethyl, propyl, 2-propyl, n-butyl, iso-butyl, tert-butyl, pentyl, hexyl, heptyl, octyl, and the like.

“Substituted alkyl” means a linear or branched saturated monovalent hydrocarbon radical of one to twenty carbon atoms, preferably 1 to 10 carbon atoms, more preferably one to six carbon atoms, which is substituted with 1 to 5 group(s), preferably 1 to 3, and more preferably 1 or 2 group(s), selected from the group consisting of hydroxy, alkoxy, acyl, acylamino, halo, thio, thioalkoxy, amido, amino, mono or disubstituted amino, carboxy, amidino, guanidino, amidoxime, sulfonylamino, cycloalkyl, heterocyclic, aryl, substituted aryl, heteroaryl, substituted heteroaryl and —NRSO₂NR′R″ (where R is hydrogen or alkyl and R′ and R″ are independently hydrogen, alkyl, haloalkyl, aryl, substituted aryl, heteroaryl, and substituted heteroaryl). Representative examples include, but are not limited to, 2-hydroxyethyl, 3-hydroxypropyl, 2-hydroxy-1-hydroxymethylethyl, 2-hydroxymethylethyl, 1-hydroxymethylethyl, 3-hydroxybutyl, 2,3-dihydroxypropyl, 1,3-dihydroxypropyl, 2-hydroxy-1-methylpropyl, 2-methoxyethyl, 3-methoxypropyl, 2-acetylethyl, 3-acetylpropyl, 2-acetylaminoethyl, 3-acetylaminopropyl, 2-aminoethyl, 3-aminopropyl, dimethylaminoethyl, dimethylaminopropyl, 2-piperidin-1-ylethyl, 2-piperazin-1-ylethyl, 3-piperazin-1-ylpropyl, 3-piperazin-1-ylpropyl, 3-amidinopropyl, 3-guanidinopropyl, 2-imidazol-2-ylethyl, 3-imidazol-2-ylpropyl, and the like.

“Alkylene” means a linear or branched saturated divalent hydrocarbon radical of one to twenty, preferably one to six carbon atoms, e.g., methylene, ethylene, 2,2-dimethylethylene, propylene, 2-methylpropylene, butylene, pentylene, and the like.

“Hydroxyalkyl” means a linear or branched saturated monovalent hydrocarbon radical of one to six carbon atoms that is substituted with 1 to 3 group, preferably 1 or 2 hydroxy group(s). Representative examples include, but are not limited to, 2-hydroxyethyl, 3-hydroxypropyl, 2-hydroxy-1-hydroxymethylethyl, 2-hydroxymethylethyl, 1-hydroxymethylethyl, 3-hydroxybutyl, 2,3-dihydroxypropyl, 1,3-dihydroxypropyl, 2-hydroxy-1-methylpropyl, and the like.

“Alkoxyalkyl” means a linear or branched saturated monovalent hydrocarbon radical of one to six carbon atoms that is substituted with 1 to 3 group, preferably 1 or 2 alkoxy group(s). Representative examples include, but are not limited to, 2-methoxyethyl, 3-methoxypropyl, 2-methoxy-1-methoxymethylethyl, 1-methoxymethylethyl, 3-methoxybutyl, 2,3-dimethoxypropyl, 1,3-dimethoxypropyl, 2-methoxy-1-methylpropyl, and the like.

“Aminoalkyl” means a linear or branched saturated monovalent hydrocarbon radical of one to six carbon atoms that is substituted with 1 to 3 group(s), preferably 1 or 2 amino group(s). Representative examples include, but are not limited to, 2-aminoethyl, 2-aminomethylethyl, 1-aminomethylethyl, 3-aminoybutyl, 2-amino-1-methylpropyl, and the like.

“Alkenyl” means a linear monovalent hydrocarbon radical of two to six carbon atoms or a branched monovalent hydrocarbon radical of three to six carbon atoms, containing at least one double bond, e.g., ethenyl, propenyl, and the like.

“Substituted alkenyl” means an alkenyl radical, as defined herein, that is substituted with 1 to 3 group(s), preferably 1 or 2 group(s) selected from the group consisting of hydroxy, alkoxy, acyl, acylamino, halo, amino, mono or disubstituted amino, carboxy, amidino, guanidino, sulfonylamino, heterocyclic, aryl, substituted aryl, heteroaryl, substituted heteroaryl, and —NRSO₂NR′R″ (where R is hydrogen or alkyl and R′ and R″ are independently hydrogen, alkyl, haloalkyl, aryl, or heteroaryl).

“Cycloalkyl” means a saturated monovalent or divalent cyclic hydrocarbon radical of three to six ring carbons, e.g., cyclopropyl, cyclopentyl, cyclohexyl, and the like. When the cycloalkene is divalent, the divalent structure is sometimes referred to herein as “cycloalkene”.

“Substituted cycloalkyl” means a cycloalkyl radical as defined herein, that is substituted with one, two or three substituents, preferably one or two substituents, independently selected from alkyl, alkoxy, substituted alkyl, acyl, acylamino, sulfonylamino, halo, nitro, cyano, amino, monosubstituted or disubstituted amino and —NRSO₂NR′R″ (where R is hydrogen or alkyl and R′ and R″ are independently hydrogen, alkyl, haloalkyl, aryl, substituted aryl, heteroaryl, and substituted heteroaryl). When the substituted cycloalkene is divalent, the divalent structure is sometimes referred to herein as “substituted cycloalkene”.

“Sulfonylamino” means a radical —NRSO₂R′ where R is hydrogen or alkyl and R′ is alkyl, substituted alkyl, amino, monosubstituted amino, disubstituted amino, aryl, substituted aryl, aralkyl, substituted aralkyl, heteroaryl, substituted heteroaryl, heteroaralkyl, and substituted heteroaralkyl e.g., methylsulfonylamino, benzylsulfonylamino, N-methylaminosulfonylamino, and the like.

“Alkoxy” means a radical —OR where R is an alkyl as defined above e.g., methoxy, ethoxy, propoxy, butoxy and the like.

“Acyl” means a radical —C(O)R, where R is hydrogen, alkyl, substituted alkyl, aryl, substituted aryl, aralkyl, substituted aralkyl, heteroaryl, substituted heteroaryl, heteroaralkyl, substituted heteroaralkyl, heterocyclic and heterocyclicalkyl group as defined herein. Representative examples include, but are not limited to formyl, acetyl, benzoyl, benzylcarbonyl, glycyl and the like.

“Acylamino” means a radical —NR′C(O)R, where R′ is hydrogen or alkyl, and R is hydrogen, alkyl, substituted alkyl, aryl, substituted aryl, aralkyl, substituted aralkyl, heteroaryl, substituted heteroaryl, heteroaralkyl, substituted heteroaralkyl, heterocyclic, and heterocyclicalkyl group as defined herein. Representative examples include, but are not limited to formylamino, acetylamino, benzoylamino, benzylcarbonylamino, and the like. Preferred acylamino groups include the following: —NHC(O)CH(NH₂)CH₃; —NHC(O)CH(NH₂)—(CH₂)₃—NH—C(NH)NH₂; —NHC(O)CH(NH₂)—CH₂—C(O)NH₂; —NHC(O)CH(NH₂)—CH₂—CO₂H; —NHC(O)CH(NH₂)—CH₂—SH; —NHC(O)CH(NH₂)—(CH₂)₂—C(O)NH₂; —NHC(O)CH(NH₂)—(CH₂)₂—CO₂H; —NHC(O)CH₂—NH₂; —NHC(O)CH(NH₂)—CH₂—(C₃H₂N₂); —NHC(O)CH(NH₂)—CH(CH₃)CH₂CH₃; —NHC(O)CH(NH₂)—CH₂CH(CH₃)₂; —NHC(O)CH(NH₂)—(CH₂)₄—NH₂; —NHC(O)CH(NH₂)—(CH₂)₂—SCH₃; —NHC(O)CH(NH₂)—CH₂Ph; —NHC(O)CH(NH₂)—(C₄H₈N); —NHC(O)CH(NH₂)—CH₂OH; —NHC(O)CH(NH₂)—CH(OH)CH₃; —NHC(O)CH(NH₂)—CH₂—(C₈H₆N); —NHC(O)CH(NH₂)—CH₂—Ph-p-OH; and, —NHC(O)CH(NH₂)—CH(CH₃)₂.

“Aminoacyl” means a radical —C(O)NHR, R is hydrogen, alkyl, alkylamino, hydroxyalkyl, aryl, aralkyl, heteroaryl, heteroaralkyl, heterocyclyl, and heterocyclicalkyl group as defined herein. Representative examples include, but are not limited to aminocarbonyl, methyllaminocarbonyl, benzylaminocarbonyl, and the like.

“Amine” or “amino” groups are represented by the formula —NR′R″ where R′ and R″ are independently hydrogen, alkyl, substituted alkyl, alkenyl, substituted alkenyl, aryl, substituted aryl, cycloalkyl, substituted cycloalkyl, heterocyclic, heteroaryl, substituted heteroaryl, and where R′ and R″, together with the nitrogen to which they are attached, form a heterocyclic or heteroaryl group.

“Monosubstituted amino” means a radical —NHR where R represents an alkyl, acyl, aryl, substituted aryl, aralkyl, substituted aralkyl, heteroaryl, substituted heteroaryl, heteroaralkyl, substituted heteroaralkyl, heterocyclic, and heterocyclicalkyl group as defined herein. Representative examples include, but are not limited to methylamino, ethylamino, phenylamino, benzylamino, and the like.

“Disubstituted amino” means a radical —NRR′ where R and R′ are independently selected from the group consisting of alkyl, acyl, aryl, aralkyl, substituted aralkyl, heteroaryl, substituted heteroaryl, heteroaralkyl, substituted heteroaralkyl, heterocyclic, and heterocyclicalkyl group as defined herein. Representative examples include, but are not limited to dimethylamino, diethylamino, ethylmethylamino, diphenylamino, dibenzylamino, and the like.

“Hydrazines” are represented by the formula —NHNR′R″ where R′ and R″ are independently hydrogen, alkyl, substituted alkyl, alkenyl, substituted alkenyl, aryl, substituted aryl, cycloalkyl, substituted cycloalkyl, heterocyclic, heteroaryl, substituted heteroaryl, and where R′ and R″, together with the nitrogen to which they are attached, form a heterocyclic or heteroaryl group.

“Amidino” groups are represented by the formula —C(═NR′″) NR′R″ where R′ R″ and R′″ are independently hydrogen, alkyl, substituted alkyl, alkenyl, substituted alkenyl, aryl, substituted aryl, cycloalkyl, substituted cycloalkyl, heterocyclic, heteroaryl, substituted heteroaryl, and where R′ and R″, together with the nitrogen to which they are attached, form a heterocyclic or heteroaryl group.

“Guanidino” groups is represented by the formula —NR″″C(═NR′″)NR′R″ where R′ R″ R′″ and R″″ are independently hydrogen, alkyl, substituted alkyl, alkenyl, substituted alkenyl, aryl, substituted aryl, cycloalkyl, substituted cycloalkyl, heterocyclic, heteroaryl, substituted heteroaryl, and where R′ and R″, together with the nitrogen to which they are attached, form a heterocyclic or heteroaryl group.

The term “amidoxime” refers to hydroxy substituted amidino groups, where amidino is as defined herein.

The term “thioalkoxy” refers to —S-alkyl and —S-substituted alkyl.

The term “aryloxy” refers to —O-Aryl and —O-substituted aryl.

The term “cycloalkylene” refers to a divalent cycloalkyl group where cycloalkyl is as defined herein.

The term “ornithylamino” refers to an amino terminated ornithine group:

—NH—C(O)—CH((CH₂)₃NH₂)(NH₂)

“polyoxyalkylene” is a group of the formula (-alk-O—)_{q}—R, where R′ is hydrogen, alkyl, cycloalkyl, aryl, heteroaryl, heterocyclickalkyl, where R is selected from the group of hydrogen, alkyl, cycloalkyl, aryl, heteroaryl, heterocyclicalkyl, where alk is selected from the group consisting of alkylene and substituted alkylene and q is an integer from 1 to 20.

“Halo” means fluoro, chloro, bromo, or iodo, preferably fluoro and chloro.

“Haloalkyl” means alkyl substituted with one or more of the same or different halo atoms, and preferably 1 to 5 halo atoms, e.g., —CH₂Cl, —CF₃, —CH₂CF₃, —CH₂CCl₃, and the like.

“Aryl” means a monovalent or divalent monocyclic or bicyclic aromatic hydrocarbon radical of 6 to 14 ring atoms e.g., phenyl, naphthyl, or anthryl, and phenylene, naphthylene, or anthrylene. When aryl is divalent, the divalent structure is sometimes referred to herein as “arylene”.

“Substituted aryl” means an aryl ring as defined above which is substituted independently with one to five substituents and preferably one, two or three substituents, and more preferably one or two substituents, selected from alkyl, alkoxy, aryloxy, substituted alkyl, acyl, acylamino, sulfonylamino, halo, nitro, cyano, amino, monosubstituted or disubstituted amino and —NRSO₂NR′R″ (where R is hydrogen or alkyl and R′ and R″ are independently hydrogen, alkyl, haloalkyl, aryl, substituted aryl, heteroaryl, and substituted heteroaryl). When substituted aryl is divalent, the divalent structure is sometimes referred to herein as “substituted arylene”.

“Heteroaryl” means a monovalent or divalent monocyclic, bicyclic or trycyclic radical of 5 to 12 ring atoms having at least one aromatic ring containing one, two, three, or four ring heteroatoms selected from N, O, or S, the remaining ring atoms being C, with the understanding that the attachment point(s) of the heteroaryl radical will be on an aromatic ring. More specifically the term heteroaryl includes, but is not limited to, pyridyl, furanyl, thienyl, tetrazolyl, thiazolyl, isothiazolyl, triazolyl, imidazolyl, isoxazolyl, pyrrolyl, pyrazolyl, pyrimidinyl, benzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, pyridylene, furanylene, thienylene, tetrazolylene, thiazolylene, isothiazolylene, triazolylene, imidazolylene, isoxazolylene, pyrrolylene, pyrazolylene, pyrimidinylene, benzofuranylene, isobenzofuranylene, benzothiazolylene, benzoisothiazolylene, benzotriazolylene, indolylene, isoindolylene, benzoxazolylene, quinolylene, tetrahydroquinolinylene, isoquinolylene, benzimidazolylene, benzisoxazolylene or benzothienylene. When heteroaryl is divalent, the divalent structure is sometimes referred to herein as “heteroarylene”.

“Substituted heteroaryl” means a heteroaryl ring as defined herein which is substituted with one, two or three substituents, preferably one or two substituents, independently selected from the group consisting of alkyl, aryloxy, alkoxy, substituted alkyl, acyl, acylamino, sulfonylamino, halo, nitro, cyano, amino, monosubstituted or disubstituted amino and —NRSO₂NR′R″ (where R is hydrogen or alkyl and R′ and R″ are independently hydrogen, alkyl, haloalkyl, aryl, substituted aryl, heteroaryl or substituted heteroaryl). When the substituted heteroaryl is divalent, the divalent structure is sometimes referred to herein as “Substituted heteroarylene”.

“Aralkyl”, “heteroaralkyl”, “substituted aralkyl”, “substituted heteroaralkyl”, mean a radical —R^{a′}R^{b′} where R^{a′} is an alkylene group and R^{b′} is a aryl or substituted aryl, heteroaryl or substituted heteroaryl group as defined herein, e.g., benzyl, pyridin-3-ylmethyl, imidazolylethyl, pyridinylethyl, 3-(benzofuran-2-yl)propyl, and the like.

“Cycloalkylalkyl” means mean a radical —R^{c}R^{d} where R^{c} is an alkylene group and R^{d} is a cycloalkyl group.

“Substituted cycloalkylalkyl” means a cycloalkylalkyl radical, where cycloalkylalkyl is defined above, in which either the alkyl ring or the alkylene chain is substituted with one, two or three substituents selected from alkyl, aryl, substituted aryl, substituted alkyl, acyl, acylamino, sulfonylamino, halo, nitro, cyano, amino, monosubstituted or disubstituted amino and —NRSO₂NR′R″ (where R is hydrogen or alkyl and R′ and R″ are independently hydrogen, alkyl, haloalkyl, aryl, substituted aryl, heteroaryl or substituted heteroaryl).

“Heterocyclic” means a saturated non-aromatic cyclic radical of 5 to 8 ring atoms in which one or two ring atoms are heteroatoms selected from NR (where R is independently hydrogen, alkyl, or heteroalkyl), O, or S(O)ₙ (where n is an integer from 0 to 2), the remaining ring atoms being C, where one or two C atoms may optionally be replaced by a carbonyl group. The heterocyclic ring may be optionally substituted independently with one, two, or three substituents selected from alkyl, alkoxy, substituted alkyl, acyl, acylamino, sulfonylamino, halo, nitro, cyano, amino, monosubstituted or disubstituted amino and —NRSO₂NR′R″ (where R is hydrogen or alkyl and R′ and R″ are independently hydrogen, alkyl, haloalkyl, aryl, substituted aryl, heteroaryl, and substituted heteroaryl). More specifically the term heterocyclic includes, but is not limited to, tetrahydropyranyl, 2,2-dimethyl-1,3-dioxolane, piperidino, N-methylpiperidin-3-yl, piperazino, N-methylpyrrolidin-3-yl, 3-pyrrolidino, morpholino, thiomorpholino, thiomorpholino-1-oxide, thiomorpholino-1,1-dioxide, pyrrolinyl, imidazolinyl, and the derivatives thereof.

“Heterocyclicalkyl” means a radical —R^{e}R^{f} where R^{e} is an alkylene group and R^{f} is a heterocyclic group as defined herein, e.g., tetrahydropyran-2-ylmethyl, 4-methylpiperazin-1-ylethyl, 3-piperidinylmethyl, 2,2-dimethyl-1,3-dioxoxolan-4-ylmethyl, benzyl, and the like.

“Optionally substituted phenyl” means a phenyl group that is substituted independently with one to five substituents and preferably one, two or three substituents, and more preferably one or two substituents, selected from alkyl, alkoxy, aryloxy, substituted alkyl, acyl, acylamino, sulfonylamino, halo, nitro, cyano, amino, monosubstituted or disubstituted amino and —NRSO₂NR′R″ (where R is hydrogen or alkyl and R′ and R″ are independently hydrogen, alkyl, haloalkyl, aryl, substituted aryl, heteroaryl, and substituted heteroaryl).

“Optionally substituted heteroaryl” means a monovalent monocyclic radical of 5 or 6 ring atoms having one or two ring heteroatoms selected from N, O, or S, the remaining ring atoms being C. The heteroaryl ring is optionally fused to a phenyl ring and is optionally substituted with one to three, preferably one or two substituents independently selected from alkyl, alkoxy, hydroxy, halo, haloalkyl, amino, and mono or disubstituted amino.

“Aminoalkylcarbonyl”, “heterocycliccarbonyl” and “cycloalkylcarbonyl” means a radical —COR^{a} where R^{a} is an alkylamino, a heterocyclic, and a cycloalkyl group respectively, as defined herein.

“Heteroarylthioalkyl” means a radical -alkylene-S—R^{a} where R^{a} is a heteroaryl group as defined herein.

“Optional” or “optionally” means that the subsequently described event or circumstance may but need not occur, and that the description includes instances where the event or circumstance occurs and instances in which it does not. For example, “heterocyclic group optionally mono- or di-substituted with an alkyl group” means that the alkyl may but need not be present, and the description includes situations where the heterocyclic group is mono- or disubstituted with an alkyl group and situations where the heterocyclic group is not substituted with the alkyl group.

“Hydroxy or amino protecting group” refers to those organic groups intended to protect oxygen and nitrogen atoms against undesirable reactions during synthetic procedures. Suitable oxygen and nitrogen protecting groups are well known in the art e.g., trimethylsilyl, dimethyl-tert-butylsilyl, benzyl, benzyloxy-carbonyl (CBZ), tert-butoxycarbonyl (Boc), trifluoroacetyl, 2-trimethylsilylethanesulfonyl (SES), and the like. Others can be found in the book by T. W. Greene and G. M. Wuts, Protecting Groups in Organic Synthesis, Second Edition, Wiley, New York, 1991, or T. W. Greene and G. M. Wuts, Protecting Groups in Organic Synthesis, Third Edition, Wiley, New York, 1999, and references cited therein.

“Amino acid” refers to any of the naturally occurring amino acids, as well as synthetic analogs (e.g., D-stereoisomers of the naturally occurring amino acids, such as D-threonine) and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). Unnatural amino acids are also known in the art, as set forth in, for example, Williams (ed.), Synthesis of Optically Active .alpha.-Amino Acids, Pergamon Press (1989); Evans et al., J. Amer. Chem. Soc., 112:4011-4030 (1990); Pu et al., J. Amer. Chem. Soc., 56:1280-1283 (1991); Williams et al., J. Amer. Chem. Soc., 113:9276-9286 (1991); and all references cited therein. The present invention includes the side chains of unnatural amino acids as well.

Compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. For example, if the R² substituent in a compound of formula (I) is 2-hydroxyethyl, then the carbon to which the hydroxy group is attached is an asymmetric center and therefore the compound of Formula (I) can exist as an (R)- or (S)-stereoisomer. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992 or 5^{th} edition J. March, John Wiley and Sons, New York, 2001).

A “pharmaceutically acceptable excipient” means an excipient that is useful in preparing a pharmaceutical composition that is generally safe, non-toxic or pharmaceutically acceptable toxicity and neither biologically nor otherwise undesirable, and includes an excipient that is acceptable for veterinary use as well as human pharmaceutical use. A “pharmaceutically acceptable excipient” as used in the specification and claims includes both one and more than one such excipient.

“Pharmaceutically acceptable acid addition salts” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Groups which form pharmaceutically acceptable acid addition salts include amines, hydrazines, amidines, guanidines, substituted aryl/heteroaryl and substituted alkyl groups that carry at least a nitrogen bearing substituent such as amino, hydrazino, amidino, guanidino and the like.

A compound of Formula (I) may act as a pro-drug. Prodrug means any compound that releases an active parent drug according to Formula (I) in vivo when such prodrug is administered to a mammalian subject. Prodrugs of a compound of Formula (I) are prepared by modifying functional groups present in the compound of Formula (I) in such a way that the modifications may be cleaved in vivo to release the parent compound. Prodrugs include compounds of Formula (I) wherein a hydroxy, amino, or sulfhydryl group in compound (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylamino-carbonyl) of hydroxy functional groups in compounds of Formula (I), and the like. “Treating” or “treatment” of a disease includes:

(1) preventing the disease, i.e. causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Anti-fungal” or “anti-bacterial” means that growth of the fungus or bacterial is inhibited or stopped.

“Anti-tumor” means the compound has the property of inhibiting the growth of tumor cells. Preferably, when the compound is contacted with a tumor cell line at a concentration of 100 μm, growth of the tumor cells is 32% or less as that of a no growth control.

“Bacteriostatic” means the compound has the property of inhibiting bacterial or fungal multiplication, wherein multiplication resumes upon removal of the active compound. For a bacteriostatic compound, preferably its minimum bacteriocidal concentration (MBC) is greater than 4× its minimum inhibitory concentration (MIC).

“Bacteriocidal” or “fungicidal” means that the compound has the property of killing bacteria or fungi. Bactericidal/fungicidal action differs from bacteriostasis or fungistasis only in being irreversible. For example, the “killed” organism can no longer reproduce, even after being removed form contact with the active compound. In some cases, the active compound causes lysis of the bacterial or fungal cell; in other cases the bacterial or fungal cell remains intact and may continue to be metabolically active. A bacteriocidal compound preferably exhibits a MBC that is less than 4× its MIC. Similarly, a fungicidal compound exhibits a minimum fungicidal concentration (MFC) that is preferably less than 4× its MIC.

“Minimum inhibitory concentration” or “MIC” refers to the minimum concentration of a compound necessary to completely inhibit growth of the organism tested. Compounds of this invention having an MIC of at least 1 mM are active in the assays described in the examples below Preferred compounds have an MIC of 500 μM, and even more preferably an MIC of 100 μM.

“dsDNA” means double stranded DNA.

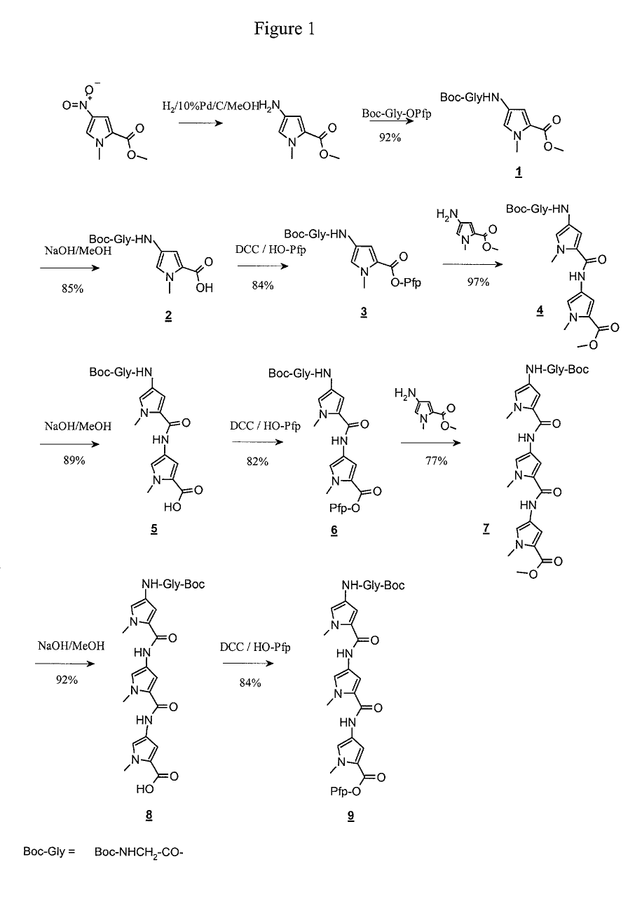

Representative compounds of this invention are shown below. FIGS. 1-7 show various ways to make these compounds.

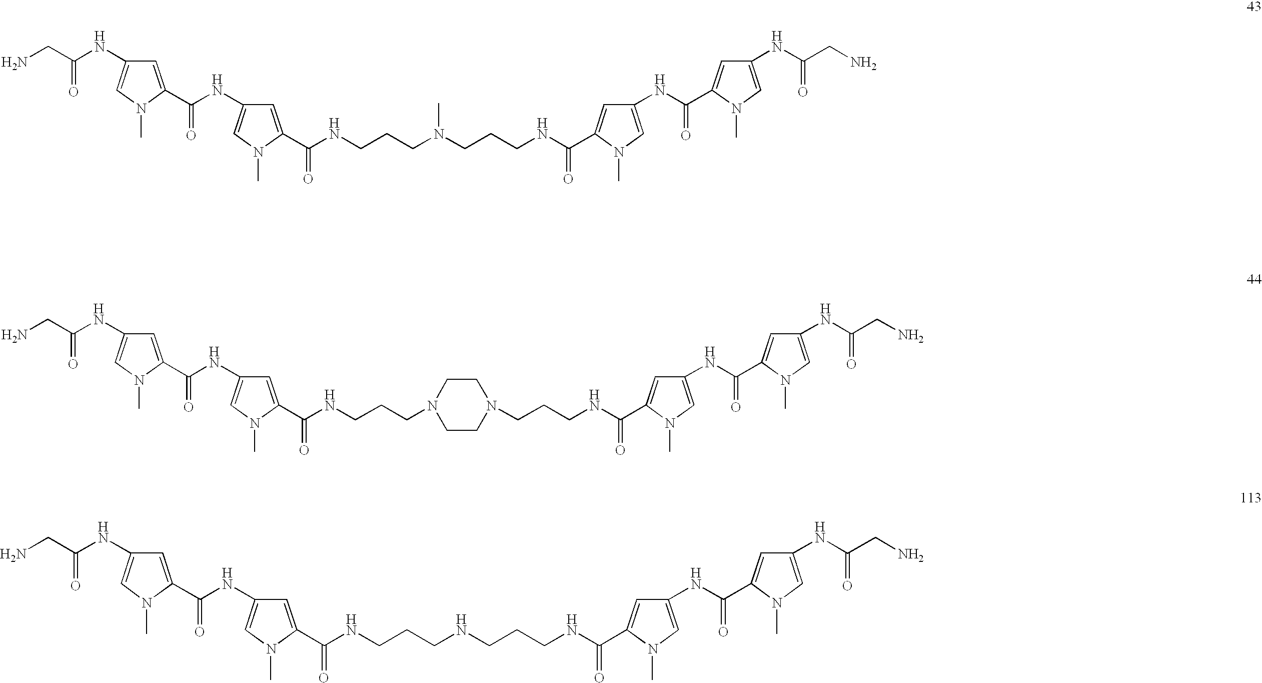

### Preferred Embodiments

While the broadest definition of this invention is set forth in the Summary of the Invention, certain compounds of Formula (I) are preferred.

1. A preferred group of compounds is that wherein Ar¹, Ar², Ar³ and Ar⁴ are independently selected from the group consisting of an optionally substituted phenyl and optionally substituted heteroaryl, preferably an optionally substituted pyrrole, indole, benzimidazole, benzofuran, or benzoxazole. More preferably, Ar¹, Ar², Ar³ and Ar⁴ are independently pyrrole, 1-alkyl pyrrole, indole, 1-alkylindole, benzimidazole, 1-alkylbenzimidazole, benzofuran, benzoxazole or 1-methylbenzoxazole wherein the pyrrole rings are attached to the amino group at the 4-position and the carbonyl group at the 2-position of the pyrrole ring and the indole, benzimidazole, benzofuran or benzoxazole rings are attached to the carbonyl group at the 2-position and the amino group at either the 5- or 6-position of the indole, benzimidazole, benzofuran or benzoxazole ring. Even more preferably, Ar¹, Ar², Ar³ and Ar⁴ are independently 1-methylpyrrole that is linked to the carbonyl group at the 2-position and the amino group at the 4-position of the pyrrole ring or indole that is attached to the carbonyl group at the 2-position and the amino group at either the 5- or 6-position of the indole ring.

2. Another preferred group of compounds is that wherein n and m are independently 0 to 3, preferably 0 to 2, more preferably 0 or 1.

3. Yet another preferred group of compounds is that wherein R¹ and R² are independently —COR³ wherein R³ is alkyl, amino, monosubstituted amino, disubstituted amino, or alkyl substituted with one, two or three substituents selected from the group consisting of amino, monosubstituted amino, disubstituted amino, guanidino, amidino, —NHCOR^{a} (wherein R^{a} is hydrogen, alkyl, aryl, aralkyl, cycloalkyl, cycloalkylalkyl, heteroaryl, heteroaralkyl, or polyoxyalkylene), —NHCONHR^{a} (wherein R^{a} is as defined above), aryl, heteroaryl, carboxy, alkoxycarbonyl, —OR^{b} (where R^{b} is hydrogen, alkyl, aryl, aralkyl, cycloalkyl, cycloalkylalkyl, heteroaryl, heteroaralkyl, or polyoxyalkylene), and polyoxyalkylene, provided that at least one of R¹ and R² is a group that can form a pharmaceutically acceptable acid addition salt.

Preferably R³ is alkyl, alkyl substituted with one, two or three substituents selected from the group consisting of amino, guanidino, amidino, —NHCOR^{a} (wherein R^{a} is hydrogen, alkyl, aryl, aralkyl, cycloalkyl, cycloalkylalkyl, heteroaryl, heteroaralkyl, or polyoxyalkylene), —NHCONHR^{a} (wherein R^{a} is as defined above), and —OR^{b} (where R^{b} is alkyl, aryl, aralkyl, cycloalkyl, cycloalkylalkyl, heteroaryl, heteroaralkyl, or polyoxyalkylene) provided that at least one of R¹ and R² is a group that can form a pharmaceutically acceptable acid addition salt. More preferably R¹ and R² are independently of each other aminomethylcarbonyl, 1-amino-4-guanidinobutylcarbonyl, 1,4-diaminobutylcarbonyl, 1,5-diaminopentylcarbonyl, 1-amino-5-(3,4-difluorophenylureido)-pentylcarbonyl, 1-(3,4-difluorophenylureido)-4-guanidinobutylcarbonyl, 1-[4-(N,N-(2-chloroethyl)aminophenyl-butanoyl)]amino-4-guanidinobutylcarbonyl, 1-amino-5-[4-(N,N-(2-chloroethyl)aminophenyl-butanoyl)]aminopentylcarbonyl, or pyrene-1-ylmethyloxy, more preferably R¹ and R² are identical and are aminomethylcarbonyl, 1-amino-4-guanidino-butylcarbonyl, 1,4-diaminobutylcarbonyl, 2-aminoethylcarbonyl or 3-aminopropylcarbonyl.

4. Yet another preferred group of compounds is that wherein L is alkylene, preferably 1,2-ethylene, 1,3-propylene, 1,4-butylene, 1,6-hexylene, 1,8-octylene, 1,12-dodecyl, 1-methylethylene, or 1,2-hexadecyl, more preferably 1,2-ethylene.

5. Yet another preferred group of compounds is that wherein L is alkylene substituted with one, two or three substituent(s) selected from the group consisting of aryl, —CONHR⁴ (wherein R⁴ is hydrogen, alkyl, hydroxyalkyl, alkoxyalkyl, aminoalkyl, aryl, aralkyl, heteroaryl, heteroaralkyl, heterocyclic, heterocyclicalkyl, heteroarylthioalkyl or —(CHR⁵)ₙ₁—CO—(NH—Ar³—CO)ₘ—NH—Ar⁴—CO—NHR³ where n1 is 1 to 3, R⁵ is hydrogen or alkyl, and Ar³, m, Ar⁴, and R³ are as defined above), —CONHNHR⁶ [wherein R⁶ is alkyl, aryl, aralkyl, —COR⁷, —COOR⁸ (wherein R⁷ and R⁸ are independently of each other alkyl, aryl, aralkyl, cycloalkyl, cycloalkylalkyl, heteroaryl or heteroaralkyl), heteroaryl, or heteroaralkyl], —NHR⁹ (wherein R⁹ is hydrogen, alkyl, aminoalkyl, hydroxyalkyl, alkoxyalkyl, aminoalkylcarbonyl, or heterocycliccarbonyl), and guanidino; preferably meso-1,2-diphenylethylene, 1-(p-nitrophenylaminocarbonyl)-1,5-pentylene, 1-(napth-2-ylaminocarbonyl)-1,5-pentylene, 1-(pentafluorophenylhydrazidocarbonyl)-1,5-pentylene, 1-(5-trifluoropyrimidin-2-ylhydrazidocarbonyl)-1,5-pentylene, 1-(2-pyrene-1-ylethylaminocarbonyl)-1,5-pentylene, 1-[2-(6-nitrobenzimidazol-1-ylethylaminocarbonyl]-1,5-pentylene, 1-[2-(indol-3-yl)-ethylaminocarbonyl]-1,5-pentylene, 1-[2-(5-fluoroindol-3-yl)ethylaminocarbonyl]-1,5-pentylene, 1-[2-(4-nitrophenyl)ethylaminocarbonyl]-1,5-pentylene, 1-(benzyloxycarbonyl-hydrazidocarbonyl)-1,2-ethylene, 1-(napth-1-ylaminocarbonyl)-1,5-pentylene, 1-(4-pyrene-1-ylbutylaminocarbonyl)-1,5-pentylene, 1-(2-(2-trifluoromethylquinolin-4-yl)thio-ethylaminocarbonyl)-1,5-pentylene, 1-(pentafluorophenylhydrazidocarbonyl)-1,4-butylene, 1-(4-pyrene-1-ylmethylaminocarbonyl)-1,5-pentylene, 1-(2-hydroxyethylaminocarbonyl)-1,5-pentylene, 1-(2-aminoethylaminocarbonyl)-1,5-pentylene, 1-(3-dimethylaminopropyl-aminocarbonyl)-1,5-pentylene, 1-(bis-(2-aminoethyl)aminoethylaminocarbonyl)-1,5-pentylene, 1-(N-(2-aminoethyl)aminoethylaminocarbonyl)-1,5-pentylene, 2-(aminomethylcarbonyl-amino)-1,3-propylene, and 2-(3-hydroxypyrrolidin-5-ylcarbonylamino)-1,3-propylene.

6. Yet another preferred group of compounds is that wherein L is -(alkylene)ₓ-Z-(alkylene)_{y}-(Z^{a})_{z}- [wherein x, y and z are independently 0, 1, or 2 and Z and Z^{a} are, independently of each other, phenylene, cycloalkylene optionally fused to one or two phenylene ring(s), heterocyclene, —O—, —S—, —NR¹⁰— [wherein R¹⁰ is hydrogen, alkyl, cycloalkylcarbonyl, hydroxyalkyl, alkoxyalkyl, aminoalkyl, —CONHR⁴, —COR⁷, —COOR⁸ (where R⁴, R⁷ and R⁸ are as defined above), —SO₂R¹¹ (where R¹¹ is alkyl, aryl, aralkyl, heteroaryl, or heteroaralkyl) or —(CHR⁵)ₙ₂—NH—(CO—Ar³—NH)ₘ—CO—Ar⁴—NHR² where n2 is 2 to 4, R⁵ is hydrogen or alkyl, and Ar³, m, Ar⁴, and R² are as defined above], —CO—NH— or —NH—CO—, provided that when Z and/or Z^{a} is —NR¹⁰— then it is separated from another nitrogen atom by at least two carbon atoms; preferably Z and Z^{a} are, independently of each other, phenylene, cycloalkylene optionally fused to one or two phenylene ring(s), heterocyclene, —NR¹⁰— [wherein R¹⁰ is hydrogen, alkyl, cycloalkylcarbonyl, hydroxyalkyl, alkoxyalkyl, aminoalkyl, —CONHR⁴, —COR⁷, —COOR⁸ (where R⁴, R⁷ and R⁸ are as defined above), —SO₂R¹¹ (where R¹¹ is alkyl, aryl, aralkyl, heteroaryl, or heteroaralkyl), —CO—NH— or —NH—CO—. Even more preferably L is m-xylene, p-xylene, m-phenylene, p-phenylene, flyorendiyl, preferably 2,7-fluorendiyl, napthalenediyl, preferably 2,7-napthalenediyl, indolediyl, preferably 2,5- or 2,6-indolediyl, benzimidazolediyl, preferably 2,5- or 2,6-benzimidazolediyl, bis-(3-N- benzyloxycarbonylamino)propylene [—(CH₂)₃—N(BzOCO—)—(CH₂)₃—], bis-(2-napth-2-ylsulfonylamino)ethylene [—(CH₂)₂—N(—SO₂napth-2-yl)—(CH₂)₂—], bis-(2-N-3,5-dinitrophenylcarbonylamino)ethylene [—(CH₂)₂—N(—CO-3,5-dinitrophenyl)—(CH₂)₂—], 1,3-cyclohexyl-bis-methylene [—(CH₂)-(1,3-C₆H₁₀)—(CH₂)—], 1,4-cyclohexyl-bis-methylene [—(CH₂)-(1,4-C₆H₁₀)—(CH₂)—], 4,4′-methylene-bis-1,4-cyclohexylene [-(1,4-C₆H₁₀)—(CH₂)-(1,4-C₆H₁₀)—], 1,2-cyclohexylene (1,2-C₆H₁₀—), bis-(2-adamantyl-1-ylcarbonylamino)ethylene, bis-(3-N-methylamino)propylene [—(CH₂)₃—N(—CH₃)—(CH₂)₃—], bis-(3-amino)propylene [—(CH₂)₃—NH—(CH₂)₃—], bis-(3-N-methylamino)propylene [—(CH₂)₃—N—(CH₃)—(CH₂)₃—], 1,4-piperazino-bis-propylene [—(CH₂)₃-(1,4-piperazino)-(CH₂)₃—], bis-(2-(2-aminoethyl)amino)ethylene [—(CH₂)₂—N(—(CH₂)₂NH₂)—(CH₂)₂—], and bis-(2-amino)ethylene [—(CH₂)₂—NH—(CH₂)₂

], particularly preferably bis-(3-N-benzyloxycarbonylamino)propylene [—(CH₂)₃—N—(—OCOC₆H₅)—(CH₂)₃—], bis-(3-aminopropylene) [—(CH₂)₃—NH—(CH₂)₃—], 2,7-fluorendiyl, 2,7-napthalenediyl, 2,5- or 2,6-indolediyl, or 2,5- or 2,6-benzimidazolediyl.

7. Yet another group of preferred compounds are N-substituted pyrrole polyamide compounds, and pharmaceutically acceptable salts, of formula (II).

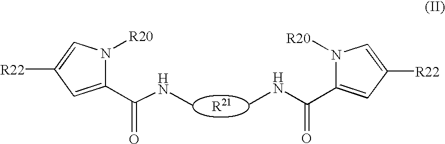

wherein R²¹ is an arylene, heteroarylene, substituted arylene or substituted heteroarylene, preferably R²¹ is 1,4-phenylene, 1,3-phenylene, substituted 1,4-phenylene, substituted 1,3-phenylene, 1,4-pyridylene, 1,3-pyridylene, 2,4-pyrimidinylene or 2,5-pyrimidinylene, 3,5-(1,2,4-)trizolene, 2,5-thiazolene, and 2,7-naphthylene; each R²⁰ is independently alkyl or substituted alkyl, more preferably independently methyl, ethyl, propyl, isoamyl or cyclopropylmethyl; and each R²² is independently guanidino or amidino. More preferred are compounds and pharmaceutically acceptable salts of the compounds represented by formulae (III), (IV), (V), and (VI) below:

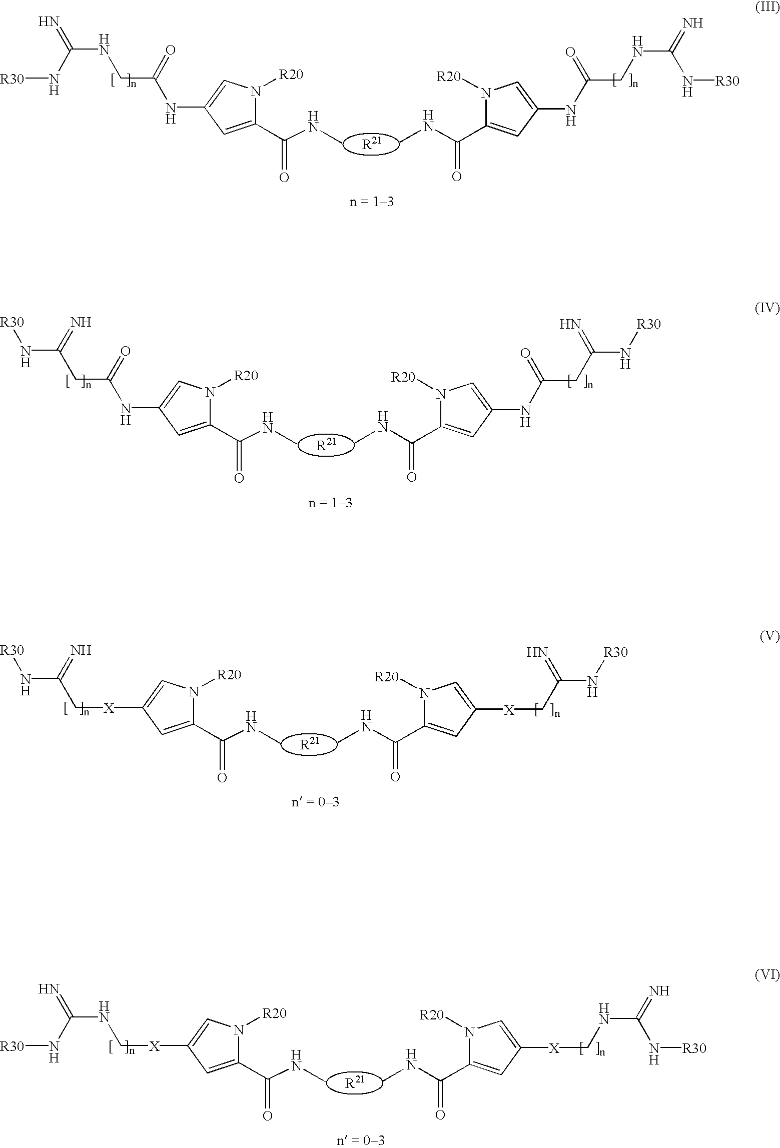

wherein each R³⁰ is independently hydrogen or alkyl or substituted alkyl, more preferably, independently hydrogen, methyl, or ethyl; and each X is independently a bond, —O— or —NH—, more preferably —NH—; n is 1 to 3 and n′ is 0-3.

8. Yet another group of preferred compounds are those of Formula (VII) below and pharmaceutically acceptable salts of these compounds:

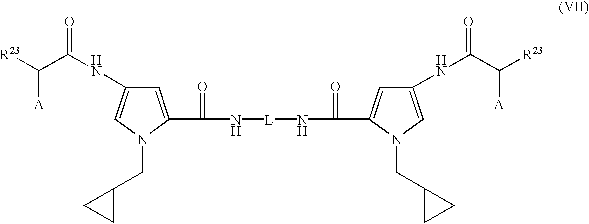

wherein L is selected from the group consisting of alkylene and cycloalkylene, more preferably, —(CH₂)₂— or 1,4 cyclohexylene. A is an amino acid side chain preferably the side chain from Gly, Val, Pro, and His, and R²³ is selected from the group consisting of guanidino, amino and ornithylamino. More preferred are compounds 160-174 and 177 below and pharmaceutically acceptable salts of these compounds.

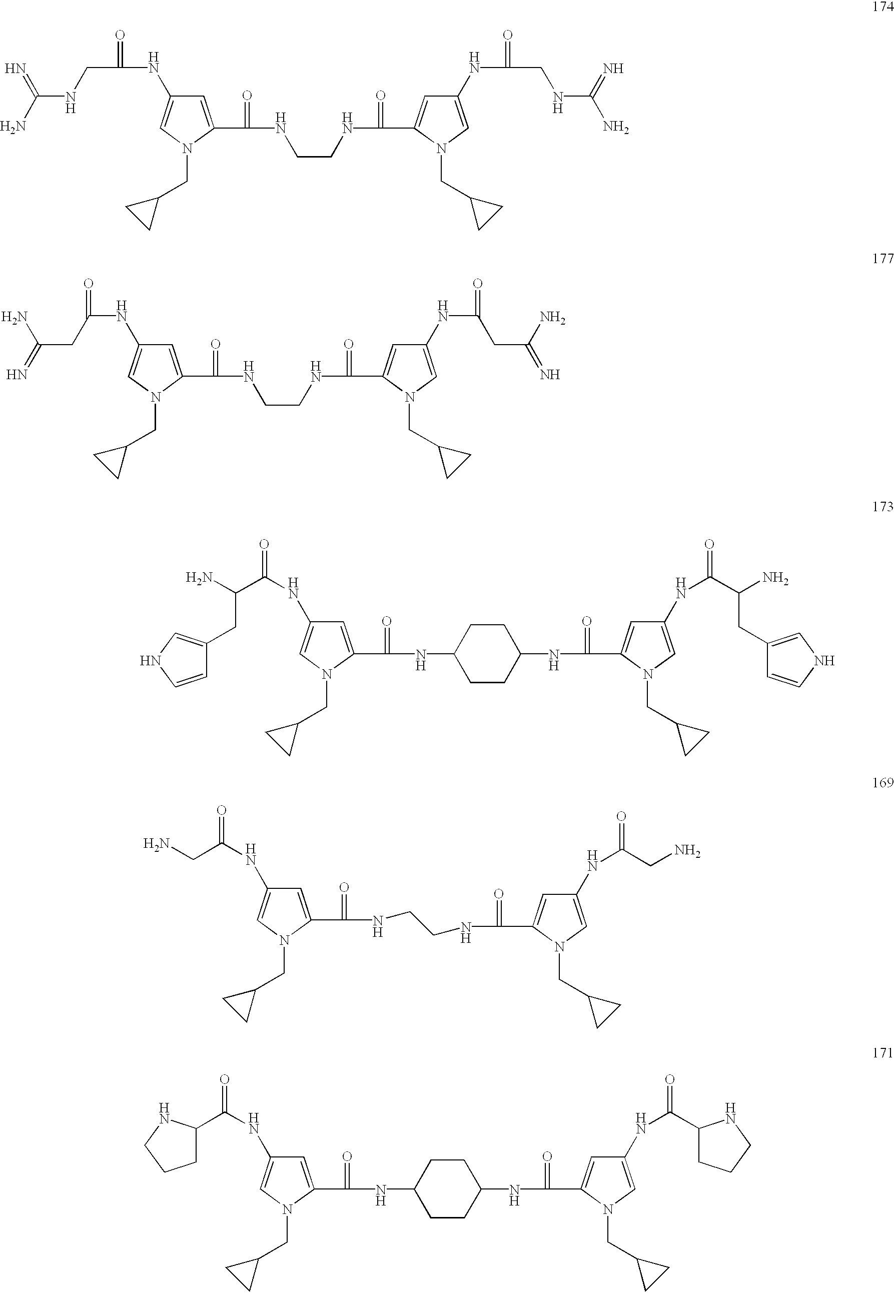

### General Synthetic Scheme

Compound of this invention can be made by the methods depicted in the reaction schemes shown below.

The starting materials and reagents in preparing these compounds are either available from commercial suppliers such as Aldrich Chemical Co., (Milwaukee, Wis., USA), Bachem (Torrance, Calif., USA), Emka-Chemie. or Sigma (St. Louis, Mo., USA) or are prepared by methods known to those skilled in the art following procedures set forth in references such as Fieser and Fieser's Reaagents for Organic Synthesis, Volumes 1-15 (John Wiley and Sons, 1991); Rodd's Chemistry of Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography, and the like. Such materials may be characterized using conventional means, including physical constants and spectral data.

### Preparation of Compounds of Formula (I)

In general, compounds of Formula (I) where Ar¹-Ar⁴, R¹ and R² are the same can be prepared as shown in Schemes A below.

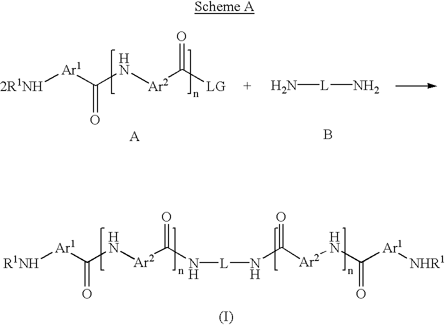

Coupling of a compound of formula A where LG is a suitable leaving group such as halo or activated alkoxy group such as pentafluorophenoxy and the like, with a diamine of formula L—(NH₂)₂ B, wherein L is as defined in the Summary of the Invention provides a compound of Formula (I). The coupling reaction is typically carried out in an inert organic solvent such as dichloromethane, dimethylformamide, and the like, and at an ambient temperature. It will be recognized by a person skilled in the art, that if compound A or B has any protecting group e.g., if R¹ and or R² are a suitable protecting group, then they would have to be removed before or after the coupling reaction to provide a compound of Formula (I).

A compound of Formula (I) can be converted to other compounds of Formula (I). For example, a compound of Formula (I) where R¹ is hydrogen can be reacted with an alkyl halide to provide a corresponding compound of Formula (I) where R¹ is alkyl. Alkyl halides are either commercially available or they can be prepared by methods well known in the art. For example, ethyl bromide, propyl bromide, butyl bromide are commercially available. A compound of Formula (I) where R¹ is hydrogen can be reacted with an acylating agent to provide a compound of Formula (I) where R¹ is —COR³ where R³ is as defined in the Summary of the Invention. The reaction is carried out in the presence of a base such as diethylisopropylamine, triethylamine and the like and in an inert organic solvent such as methylene chloride, tetrahydrofuran and the like. Acylating agent such as acetic anhydride and succinic anhydride are commercially available. Compounds of Formula (I) where R¹ is —COR³ where R³ is an alkyl substituted with groups such as aryl, heteroaryl, amino, carboxy, alkoxycarbonyl can be prepared by treating a compound of Formula (I) where R¹ is hydrogen with a commercially available N-protected amino acids such as glycine, alanine, glutamic acid, glutamine, aspartic acid, arginine, histidine, serine, and lysine followed by deprotection of the amino group.

Compounds of Formula (I) where Ar¹, Ar², and R¹ are the same or different from Ar³, Ar⁴, and R² can be prepared as shown in Schemes B below.

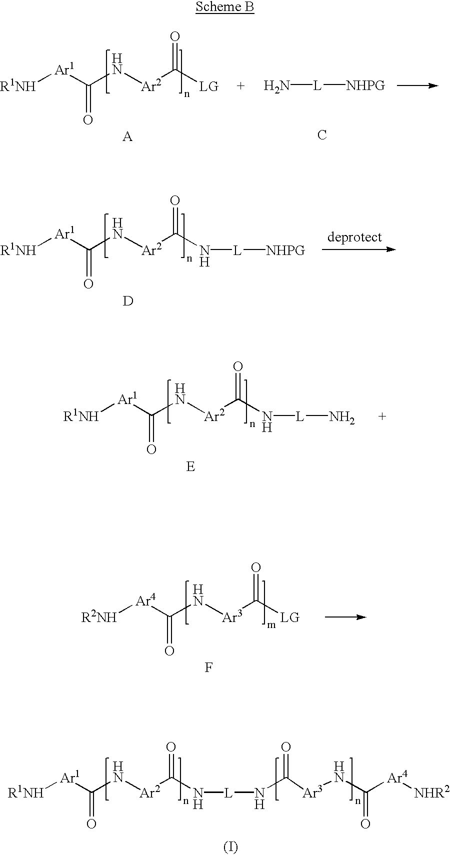

Alternatively, a compound of Formula (I) can be prepared in a sequential manner as illustrated in Scheme B above. Briefly, coupling of a compound of formula A with a monoamino-protected diamine of formula C wherein L is as defined in the Summary of the Invention provides a compound of formula D which after deprotection of the amino protecting group provides a compound of formula E. The reaction conditions utilized for amino group deprotection will depend on the type of protecting group. For example, if the protecting group is tert-butoxycarbonyl, it would be removed under acidic hydrolysis reaction conditions. Coupling E with a compound of formula F under conditions described above provides a compound of Formula (I).

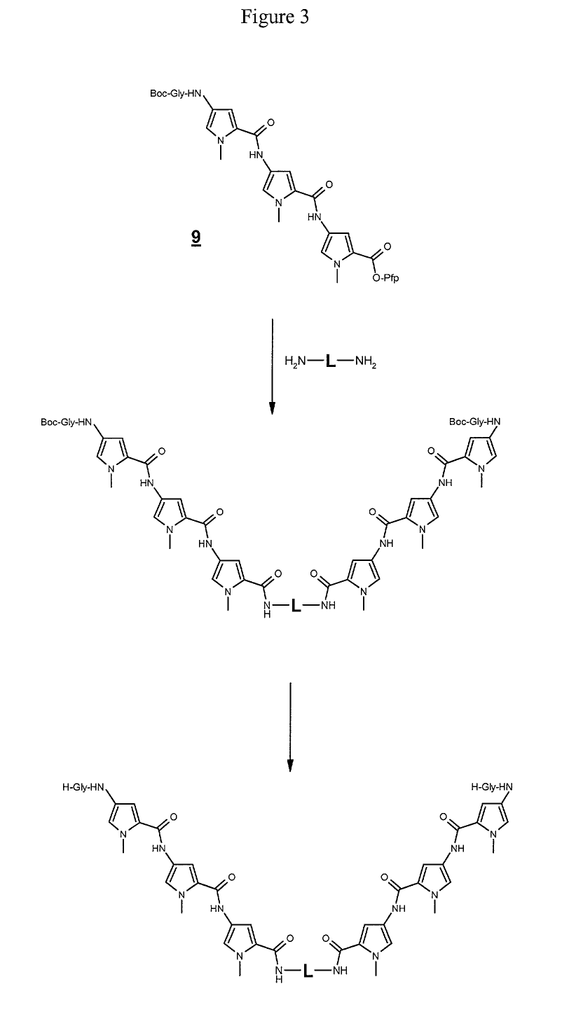

It will also be readily apparent to a person skilled in the art that a compound of Formula (I) can be converted to other compounds of Formula (I). For example, compound (I) where R¹ and/or R² are hydrogen can be converted to corresponding compounds of Formula (I) where they are not hydrogen as shown in FIG. 3.

Compounds of formula A, B, C, and D are commercially available or they can be prepared by methods well known in the art. For example, diamines of formula B such as ethylenediamine, propanediamine, octanediamine, hexadecanediamine, 1,3-, 1,4-cyclohexane(bis-methylamine), 4,4′-methylenebis(cyclohexylamine), lysinamide, 2,7-diaminofluorene, m-, p-xylenediamine, lysine beta-naphthylamide, L-lysyl pentafluorophenylhydrazide, L-lysine (4-trifluoromethylpyrimidin-2-yl)hydrazide, L-lysine 2-(pyrene-1-yl)ethylamide are commercially available. Compounds of formula C can be prepared from these commercially available diamines by any of several methods known to those skilled in the art.

Compounds of formula A used in the reactions described herein are readily prepared using, for example, the procedures illustrated below. In general, the methodology involves the formation of peptide linkages between amino acids. Because of their amino and carboxy groups, and frequently the presence of other reactive groups, it may be necessary to protect the groups and/or the activation of such groups, particularly the carboxy group, in order to achieve a certain reaction or to optimize such a reaction.

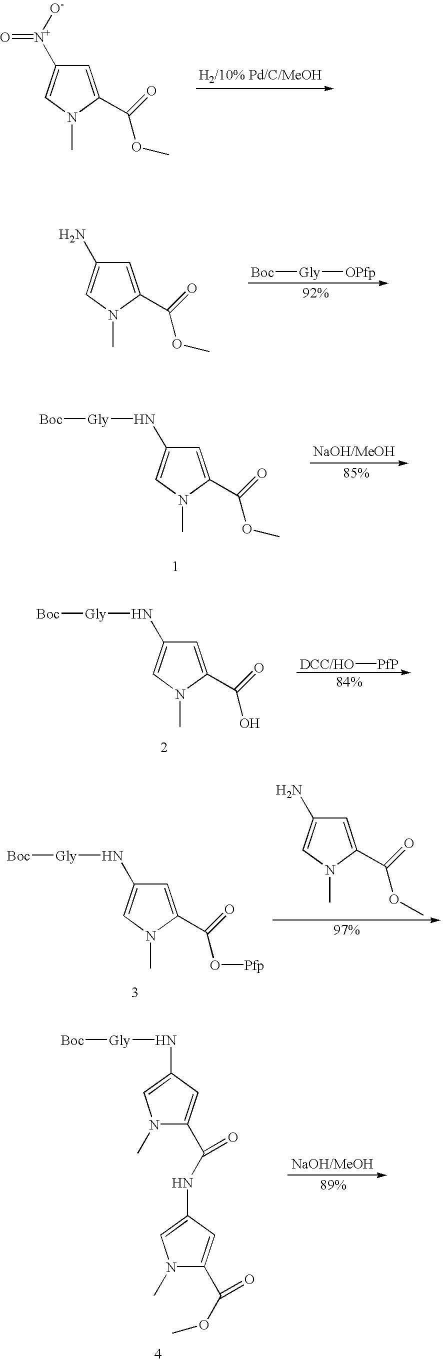

The above scheme illustrates the synthesis of Boc-Gly-Py-Py-OPfp 6 (wherein Gly=—NHCH²CO— and Py=1-methylpyrrole group) and Boc-Gly-Py-Py-Py-OPfp 9 (compound A in Schemes A and B above) used to generate compounds of Formula (I). Briefly, methyl 4-amino-1-methyl-1H-pyrrole-2-carboxylate, obtained by hydrogenation of methyl 4-nitro-1-methyl-1H-pyrrole-2-carboxylate, is converted into Boc-Gly-Py-OMe (compound 1, as in Example 1, infra), saponified to free acid Boc-Gly-Py-OH (compound 2, Example 1). Compound 2 is then converted into pentafluorophenyl ester Boc-Gly-Py-OPfp (compound 3, Example 1) which is then converted into of Boc-Gly-Py-Py-OMe 4 by the reaction with methyl 4-amino-1-methyl-1H-pyrrole-2-carboxylate (Example 1). Compound 4 is converted into Boc-Gly-Py-Py-OPfp 6 in the same way as compound 1 was converted into Boc-Gly-Py-OPfp (Example 5-6). In a similar way Boc-Gly-Py-Py-Py-OPfp (compound 4) is obtained. These reactions are exemplified in Examples 7, 8 and 9 below.

In the examples presented herein, certain protecting and activating groups are specifically illustrated. However, one skilled in the art will recognize that other protecting or activating groups could have been used. The choice of a particular protecting group is dependent to a great extent upon the availability of the necessary reagent, its effect upon solubility of the “protected” compound, its ease of removal and the presence of other groups which might be effected by its use; i.e., its selectivity, or its removal. For example, it will be necessary, or at least desirable, in many reactions to protect the amino groups and/or the carboxy groups. The synthetic route chosen for the peptide synthesis may require removal of one or the other or both of said protecting groups in order to permit further reaction at the regenerated amino or carboxy group; i.e., the protecting groups used are reversible and, in most instances, are removable independently of each other. Additionally, the choice of protecting group for a given amino group depends upon the role of said amino group in the overall reaction scheme.

Amino protecting groups having varying levels of liability, i.e., ease of removal, will be used. The same is true as regards carboxy protecting groups. Such groups are known in the art and attention is directed to the reviews by Bodansky et al., “Peptide Synthesis”, 2nd Ed., John Wiley , Sons, N.Y. (1976); Greene, “Protective Groups in Organic Synthesis”, John Wiley , Sons, N.Y. (1981); McOmie, “Protective Groups in Organic Chemistry”, Plenum Press, N.Y. (1973); and to Sheppard in “Comprehensive Organic Chemistry, The Synthesis and Reactions of Organic Compounds”, Pergaman Press, N.Y. (1979), edited by E. Haslam, Part 23.6, pages 321-339.

Conventional amino and carboxy protecting groups are known to those skilled in the art. Representative amino protecting groups, but by no means limiting thereof, are the following: such as benzyloxycarbonyl; substituted or unsubstituted aralkyl such as benzyl, trityl, benzhydryl and 4-nitrobenzyl; benzylidene; arylthio such as phenylthio, nitrophenylthio and trichlorophenylthio; phosphoryl derivatives such as dimethylphosphoryl and O,O-dibenzylphosphoryl; trialkylsilyl derivatives such as trimethylsilyl; and others as are described in U.S. Pat. No. 4,322,341 and which are incorporated herein by reference. The preferred amino protecting group is benzyloxycarbonyl. Procedures for substituting said group on a given amino group are well known. In general they comprise acylating the appropriate amino compound with benzyloxycarbonyl chloride (benzylchloroformate) in a reaction inert solvent, e.g., water, methylene chloride, tetrahydrofuran, in the presence of a base (acid acceptor) e.g., sodium or potassium hydroxide when water is solvent; and, when an organic solvent is used, in the presence of a tertiary amine such as C. sub. 1-4 trialkylamines and pyridine. When an aqueous solvent system is used the pH of the reaction is held at about pH 8-10, and preferably at pH 9. Alternatively, when the reactant; i.e., the compound, an amino group of which is to be protected, contains basic groups, it can serve as acid acceptor.

Representative carboxy protecting groups are various esters such as silyl esters, including trialkyl silyl esters, trihalosilyl esters and haloalkylsilyl esters; certain hydrocarbyl esters such as C₁₋₄ alkyl, especially t-butyl groups, benzyl and substituted benzyl esters, benzhydryl and trityl; phenacyl and phthalimidomethyl esters; certain substituted hydrocarbyl esters such as chloromethyl, 2,2,2-trichloroethyl, cyanomethyl; tetrahydropyranyl; methoxymethyl; methylthiomethyl; and others as are described in U.S. Pat. No. 4,322,341 and which are incorporated herein by reference. The protected amino is converted to the unprotected amino group by procedures known to those skilled in the art. The t-butoxycarbonyl group is readily removed by treatment will dioxane saturated with hydrogen chloride. Activation of carboxy groups as a means of expediting a given reaction is methodology known to those skilled in the art. Especially useful in the herein described reaction sequence are the use of activated esters, such as those derived from pentafluorophenol which is used in peptide syntheses.

The activated pentafluorophenoxy ester expedites subsequent reactions at said activated ester group. As the skilled artisan will recognize other activating groups could be used such as N-hydroxyphthalimido group. In both instances, a dehydrative coupling agent is used to form the activated ester. Representative of such coupling agents are 1-cyclohexyl-3-(2-morpholinoethyl)-carbodiimide metho-p-toluene sulfonate, dicyclohexyl carbodiimide, N,N′-carbonyldiimidazole, N-(3-dimethylaminopropyl)-N′-ethylcarbodiimide hydrochloride, ethoxyacetylene, diphenylketene and N-ethyl-5-phenylisoxazolene-3′-sulfonate. The reaction conditions for using such coupling agents are well described in the literature. In general they comprise the use of a reaction inert solvent and temperatures ranging from ambient to 100° C. The above-mentioned carbodiimide reagents are favored since they permit use of ambient reaction temperature and afford satisfactory yields of the desired esters. Upon completion of the coupling reactions leading to the final products, the various protecting groups can be removed by the appropriate techniques previously discussed, and the desired compounds are isolated.

Alternate methods of preparing compounds of Formula (I) are illustrated in FIGS. 1-14 below and are described in detail in working examples.

### Utility, Testing, and Administration

Utility

The compounds of present invention are antimicrobial, antifungal, antiparasitic and anti-neoplastic agents. Accordingly, the compounds and compositions containing them are therefore useful in the treatment of bacterial, fungal, and/or parasitic infection(s). The compounds and compositions containing them are therefore useful in the treatment of cancer.

Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of Formula (I) may range from approximately 0.05 to 50 mg per kilogram body weight of the recipient per day; preferably about 0.01-25 mg/kg/day, more preferably from about 0.5 to 10 mg/kg/day. Thus, for administration to a 70 kg person, the dosage range would most preferably be about 35-70 mg per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen which can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract for the treatment of diseases such as asthma and similar or related respiratory tract disorders (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist which is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of Formula (I) in combination with at least one pharmaceutically acceptable excipient. Acceptable excipients are non-toxic or have pharmaceutically acceptable toxicity, aid administration, and do not adversely affect the therapeutic benefit of the compound of Formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of Formula (I) based on the total formulation, with the balance being one or more suitable pharmaceutical excipients. Preferably, the compound is present at a level of about 1-80 wt %. Representative pharmaceutical formulations containing a compound of Formula (I) are described below.

### EXAMPLES

The following preparations and examples are given to enable those skilled in the art to more clearly understand and to practice the present invention. They should not be considered as limiting the scope of the invention, but merely as being illustrative and representative thereof. The following abbreviations are employed:

| | |
|---|---|
| AcOEt = | ethylacetate; |
| Arg = | a arginine amino acid residue; |
| Boc = | a tert-butoxycarbonyl protecting group; |
| Bt = | benzotriazolyl radical; |
| t-Bu = | a tert-butyl protecting group; |
| Bzl = | a benzyl protecting group; |
| DCC = | N,N′-dicyclohexyl carbodiimide; |
| DCE = | 1,2-dichloroethane; |
| DCM = | dichloromethane; |
| DCU = | N,N′-dicyclohexylurea; |
| Dde = | a 1-(4,4-dimethyl-2,6-dioxocyclo- |
| | hexylidene)ethyl protecting group; |
| DIEA = | diisopropylethylamine; |
| DMF = | dimethylformamide; |
| DMSO = | dimethylsulfoxide; |
| Et = | an ethyl radical; |
| EtOH = | ethanol; |
| Fmoc = | a fluorenylmethoxycarbonyl |
| | protecting group; |
| Gly = | a glycine amino acid residue; |
| HATU = | O-(7-azabenzotriazol-l-yl)- N,N,N′,N′- |
| | tetramethyluronium hexafluorophosphate |
| HBTU = | O-benzotriazol-1-yl-N,N,N′,N′-tetra- |
| | methyluronium hexafluorophosphate; |
| HMBA resin = | a hydroxymethylbenzoic acid-modified |
| | polystyrene resin; |
| HMPS resin = | a hydroxymethyl-modified polystyrene |
| | resin; |
| HOAt = | 1-hydroxy-7-azabenzotriazole; |
| HOBt = | 1-hydroxybenzotriazole; |
| HPLC = | high pressure liquid chromatography; Lys |
| | a lysine amino acid residue; |
| Me = | a methyl radical; |
| MBHA resin = | a methylbenzhydrylamine resin; |
| MeOH = | methanol; |
| MMT = | a monomethoxytrityl (p-anisyl- |
| | diphenylmethyl) protecting group; |
| MS = | mass spectrum; |
| Mts = | a mesitilene-2-sulfonyl protecting group; |
| mp = | melting point; |
| mp d = | melting point with decomposition; |
| NMP = | N-methyl-2-pyrrolidinone; |
| NMR = | nuclear magnetic resonance spectrum; |
| Np = | a 4-nitrophenyl radical; |
| Pyr = | pyridine; |
| TFA = | trifluoroacetic acid; |
| TFE = | 2,2,2-trifluoroethanol; |
| THF = | tetrahydrofuran; |
| TLC = | thin layer chromatography on silica gel; |
| Pfp = | a pentafluorophenyl radical; |
| Phe = | a phenyl radical; |
| PS resin = | a polystyrene resin; |
| Py = | a 4-amino-1-methyl-1H-pyrrole-2- |
| | carboxylic acid residue; |
| SA-But-AM resin = | a 4-sulfamylbutyryl aminomethyl- |
| | polystyrene resin; |
| Wang resin = | a 4-alkoxybenzyl alcohol-modified |
| | polystyrene resin; |
| Z = | a benzyloxycarbonyl protecting group; |

In reporting NMR data, chemical shifts are given in ppm and coupling constants (J) given in Hertz (Hz). All melting points are uncorrected. All temperatures are in degrees Celsius (25° C. refers to ambient or room temperature). All parts not otherwise indicated are by weight, except for mixtures of liquids, which are by volume.

All chemicals used were of reagent grade. Melting points were determined on Mel-temp apparatus (Laboratory Devices, Inc.) and are uncorrected. ¹H NMR spectra were recorded on Varian Mercury VX-300 MHz. Chemical shifts are reported in ppm relative to the solvent residual signal. The samples were prepared in methylsulfoxide-d₆ unless otherwise specified. The peaks were assigned based on gCOSY experiments. Electrospray mass spectra were recorded on a spectrometer Mariner-EMS (PE-Biosystems). HPLC-purification were carried out on a Vydac 12 μm C₁₈ (2.2×25 cm) column using a solvent gradient with two solvents: 0.1% TFA in water (A) and 0.1% TFA in acetonitrile (B). Unless otherwise stated, the applied conditions for purification were 10% to 70% eluent B gradient over 40 minutes with a flow rate of 10 mL/min. The monitoring was at 254 nm.

### Example 1

### Preparation of Methyl 4-(Boc-NHCH₂CONH)-1-methyl-1H-pyrrole-2-carboxylate 1 (Following Figure I)

Step 1

To a stirred solution of methyl 4-nitro-1-methyl-1H-pyrrole-2-carboxylate (5.34 g, 29.0 mmole) in a mixture of AcOEt/MeOH (1/1) (100 ml) was added 10% Pd/C (Degauss type, Aldrich) (1.0 g). The flask was evacuated and then flushed 3 times with hydrogen and finally filled with hydrogen at 40 to 50 psi. The filtrate was stirred vigorously at 23° C. for 1 hour. The suspended material was filtered off through a pad of Celite in a Buchner funnel and then the funnel was rinsed several times with a small portion of AcOEt and MeOH. The combined filtrate and washings was evaporated in vacuo to dryness. The resulted methyl 4-amino-1-methyl-1H-pyrrole-2-carboxylate was used for the next step without purification.

Step 2

A solution of Boc-Gly-OPfp (10.0 g, 29.3 mmole) and freshly prepared (as described above) 4-amino-1-methyl-1H-pyrrole-2-carboxylate (29.0 mmole) in dry dioxane was kept at ambient temperature for 15 hours and evaporated. The reaction mixture was dissolved in AcOEt (400 ml) and washed successively with 5% NaHCO₃ (3×100 ml), brine (1×100 ml), 0.01M ice cold sulfuric acid (3×100 ml) and brine (3×100 ml), dried (MgSO₄), and evaporated in vacuo to dryness. The residue was crystallized from AcOEt/hexane to give 5.10 g (56.5%) of methyl 4-(Boc-NHCH₂CONH)-1-methyl-1H-pyrrole-2-carboxylate 1 as white crystals. The mother liquor was evaporated and the residue was chromatographed over a silica gel column (5.0×20 cm) using 30% AcOEt/toluene as eluent to give another 3.20 g (35.4%). The overall yield was 8.30 g (91.9%).

¹H-NMR (DMSO-d₆): δ 9.82 (s, 1H, —C(═O)NH-Py-); 7.31, 6.73 (d, d, 1H, 1H, H-3,H-5, Py); 7.00 (t, 1H, Boc-NHCH₂); 3.79 (s, 3H, NCH₃, Py); 3.70 (s, 3H, —COOCH₃, Py); 3.61 (d, 2H, Boc-NHCH₂); 1.36 (s, 9H, Boc).

### Example 2

### Preparation of 4-(Boc-NHCH₂CONH)-2-carboxy-1-methyl-1H-pyrrole 2 (Following FIG. 1)

A solution of 1 (8.0 g, 25.7 mmole) in MeOH (100 ml), containing 5N NaOH (20 ml, 100 mmole), was stirred for 5 h at 60C and evaporated. The residue was dissolved in water (200 ml) and acidified with 1N HCl up to pH 3.5. The yellowish precipitate was collected, washed with water (5×10 ml) and dried in vacuo over phosphorus pentoxide to give 4-(Boc-NHCH₂CONH)-2-carboxy-1-methyl-1H-pyrrole 2 6.5 g (85%).

¹H-NMR (DMSO-d₆): δ 12.11 (bs, 1H, —COOH, Py); 9.79 (s, 1H, —C(═O)NH-Py-); 7.26, 6.67 (s, s, 1H, 1H, H-3,H-5, Py); 6.99 (t, 1H, Boc-NHCH₂); 3.77 (s, 3H, NCH₃, Py); 3.60 (d, 2H, Boc-NHCH₂); 1.36 (s, 9H, Boc).

### Example 3

### Preparation of Pentafluoro 4-(Boc-NHCH₂CONH)-1-methyl-1H-pyrrole-2-carboxylate 3 (Following FIG. 1)

A solution of 2 (37.5 g, 126.1 mmole), pentafluorophenol (24.0 g, 130 mmole) and DCC (26.8 g, 130 mmole) in DMF (300 ml), was stirred for 18 h at ambient temperature. DCU was filtered off and discarded. The filtrate was evaporated. The residue was crystallized from benzene to give pentafluorophenyl 4-(Boc-NHCH₂CONH)-1-methyl-1H-pyrrole-2-carboxylate 3 49.0 g (84%) as a white crystalline material.

¹H-NMR (DMSO-d₆): δ 9.79 (s, 1H, —C(═O)NH-Py-); 7.26, 6.67 (s, s, 1H, 1H, H-3,H-5, Py); 6.99 (t, 1H, Boc-NHCH₂); 3.77 (s, 3H, NCH₃, Py); 3.60 (d, 2H, Boc-NHCH₂); 1.36 (s, 9H, Boc).

¹⁹F-NMR (DMSO-d₆): δ −153.65 (m, 2F, F-2,F-6,-OPfp); −158.33 (m, 1F, F-4, -OPfp); −162.44 (m, 2F, F-3, F-5, -OPfp).

### Example 4

### Preparation of Methyl 4-[4-(Boc-NHCH₂CONH)-1-methyl-1H-pyrrol-2-ylcarbonylamino]-1-methyl-1H-pyrrole-2-carboxylate 4 (Following FIG. 1)

Step 1

To a stirred solution of methyl 4-nitro-1-methyl-1H-pyrrole-2-carboxylate (20.26 g, 110 mmole) in a mixture of AcOEt/EtOH (3/2) (250 ml) was added 10% Pd/C (Degussa type, Aldrich) (1.0 g). The flask was evacuated and then flushed 3 times with hydrogen and finally filled with hydrogen at 40 to 50 psi. The resultant suspension was stirred vigorously at 23° C. for 1 hour. The suspended material was filtered off through a pad of Celite in a Buchner funnel and then the funnel was rinsed several times with a small portion of AcOEt and EtOH. The combined filtrate and washings was evaporated in vacuo to dryness. The resulted methyl 4-amino-1-methyl-1H-pyrrole-2-carboxylate was used for the next step without purification.

Step 2

A solution of pentafluorophenyl 4-(Boc-NHCH₂CONH)-1-methyl-1H-pyrrole-2-carboxylate 3 (49.0 g, 105.7 mmole) and freshly prepared (as described above) 4-amino-1-methyl-1H-pyrrole-2-carboxylate (110.0 mmole) in dry DMF was kept at ambient temperature for 72 hours and evaporated. The residue was co-evaporated with toluene (3×200 ml) and crystallized from toluene to give 44.5 g (97%) of methyl 4-[4-(Boc-NHCH₂CONH)-1-methyl-1H-pyrrol-2-ylcarbonylamino]-1-methyl-1-pyrrole-2-carboxylate 4 as white crystalline material.

¹H-NMR (DMSO-d₆): δ 9.87, 9.81 (s, s, 1H, 1H, —NHCH₂C(═O)NH-Py-, PyC(═O)NH-Py-); 7.42, 7.14, 6.89, 6.88 (d, d, d, d, 1H, 1H, 1H, 1H, H-3, H-5, Py₁, Py₂); 6.99 (t, 1H, Boc-NHCH₂); 3.81 (s, 6H, NCH₃, Py₁, Py₂); 3.70 (s, 3H, —COOCH₃, Py₂); 3.64 (d, 2H, Boc-NHCH₂); 1.38 (s, 9H, Boc).

### Example 5

### Preparation of 4-[4-(Boc-NHCH₂CONH)-1-methyl-1H-pyrrol-2-ylcarbonylamino]-1-methyl-1H-pyrrole-2-carboxylic Acid (Following FIG. 1)

A solution of methyl 4-[4-(Boc-NHCH₂CONH)-1-methyl-1H-pyrrol-2-ylcarbonyl-amino]-1-methyl-1H-pyrrole-2-carboxylate (21.67 g, 50.0 mmole) in MeOH (300 ml), containing 5N NaOH (40 ml, 200 mmole), was stirred for 5 h at 60C and evaporated. The residue was dissolved in water (300 ml) and acidified with 1N HCl up to pH 3.5. The yellowish precipitate was collected, washed with water (5×10 ml) and dried in vacuo over phosphorus pentoxide to give 4-[4-(Boc-NHCH₂CONH)-1-methyl-1H-pyrrol-2-yl-carbonylamino]-1-methyl-1H-pyrrole-2-carboxylic acid 5 18.66 g (89%).

¹H-NMR (DMSO-d₆): δ 9.85, 9.81 (s, s, 1H, 1H, —NHCH₂C(═O)NH-Py-, PyC(═O)NH-Py-); 7.39, 7.13, 6.87, 6.80 (d, d, d, d, 1H, 1H, 1H, 1H, 1H, H-3, H-5, Py₁, Py₂); 6.99 (t, 1H, Boc-NHCH₂); 3.81, 3.80 (s, s, 3H, 3H, NCH₃, Py₁, Py₂); 3.64 (d, 2H, Boc-NHCH₂); 1.38 (s, 9H, Boc).

### Example 6

### Preparation of Pentafluorophenyl 4-[4-(Boc-NHCH₂CONH)-1-methyl-1H-pyrrol-2-ylcarbonylamino]-1-methyl-1H-pyrrole-2-carboxylate 6 (Following FIG. 1)

A solution of 4-[4-(Boc-NHCH₂CONH)-1-methyl-1H-pyrrol-2-ylcarbonylamino]-1-methyl-1H-pyrrole-2-carboxylic acid 5 (15.3 g, 36.5 mmole), pentafluorophenol (7.36 g, 40 mmole) and DCC (8.24 g, 40 mmole) in DMF (150 ml), was stirred for 18 h at ambient temperature. DCU was filtered off and discarded. The filtrate was evaporated. The residue was co-evaporated with toluene (3×200 ml) and crystallized from toluene to give 17.52 g (82%) of pentafluorophenyl 4-[4-(Boc-NHCH₂CONH)-1-methyl-1H-pyrrol-2-ylcarbonyl-amino]-1-methyl-1H-pyrrole-2-carboxylate 6 as white crystalline material.

¹H-NMR (DMSO-d₆): δ 10.05, 9.85 (s, s, 1H, 1H, —NHCH₂C(═O)NH-Py-, PyC(═O)NH-Py-); 7.73, 7.28, 7.18, 6.95 (d, d, d, d, 1H, 1H, 1H, 1H, H-3, H-5, Py₁, Py₂); 7.02 (t, 1H, Boc-NHCH₂); 3.89, 3.85 (s, s, 3H, 3H, NCH₃, Py₁, Py₂); 3.66 (d, 2H, Boc-NHCH₂); 1.40 (s, 9H, Boc).

¹⁹F-NMR (DMSO-d₆) : δ −153.65 (m, 2F, F-2, F-6, -OPfp); −158.33 (m, 1F, F-4, -OPfp); −162.44 (m, 2F, F-3, F-5, -OPfp).

### Example 7

### Preparation of Methyl 4-{4-[(Boc-NHCH₂CONH)-1-methyl-1H-pyrrol-2-ylcarbonylamino]-1-methyl-1H-pyrrol-2-ylcarbonylamino]-1-methyl-1H-pyrrole-2-carboxylate 7 (Following FIG. 1)

Step 1

To a stirred solution of methyl 4-nitro-1-methyl-1H-pyrrole-2-carboxylate (4.05 g, 22.0 mmole) in a mixture of AcOEt/EtOH (3/2) (100 ml) was added 10% Pd/C (Degussa type, Aldrich) (0.5 g). The flask was evacuated and then flushed 3 times with hydrogen and finally filled with hydrogen at 40 to 50 psi. The resultant suspension was stirred vigorously at 23° C. for 1 hour. The suspended material was filtered off through a pad of Celite in a Buchner funnel and then the funnel was rinsed several times with a small portion of AcOEt and EtOH. The combined filtrate and washings was evaporated in vacuo to dryness. The resulted methyl 4-amino-1-methyl-1H-pyrrole-2-carboxylate was used for the next step without purification.

Step 2

A solution of pentafluorophenyl 4-[4-(Boc-NHCH₂CONH)-1-methyl-1H-pyrrol-2-ylcarbonyl-amino]-1-methyl-1H-pyrrole-2-carboxylate 6 (11.7 g, 20.0 mmole) and freshly prepared (as described above) 4-amino-1-methyl-1H-pyrrole-2-carboxylate (22.0 mmole) in dry DMF (50 ml) was kept at ambient temperature for 72 hours and evaporated. The residue was co-evaporated with toluene (3×200 ml) and crystallized from toluene/hexane to give 8.56 g (77%) of methyl 4-{4-[(Boc-NHCH₂CONH)-1-methyl-1H-pyrrol-2-ylcarbonylamino]-1-methyl-1H-pyrrol-2-ylcarbonylamino]-1-methyl-1H-pyrrole-2-carboxylate 7 as yellowish crystalline material.

¹H-NMR (DMSO-d₆): δ 9.92, 9.91, 9.82 (s, s, s, 1H, 1H, 1H, —NHCH₂C(═O)NH-Py₁-, Py₁-C(═O)NH-Py₂-, Py₂-C(═O)NH-Py₃-); 7.45, 7.22, 7.14, 7.04, 6.89, 6.88 (d, d, d, d, d, d, 1H, 1H, 1H, 1H, 1H, 1H, H-3, H-5, Py₁, Py₂, Py₃); 7.00 (t, 1H, Boc-NHCH₂); 3.82 (s, 9H, NCH₃, Py₁, Py₂); 3.71 (s, 3H, —COOCH₃, Py₃); 3.63 (d, 2H, Boc-NHCH₂); 1.37 (s, 9H, Boc).

### Example 8

### Preparation of 4-{4-[(Boc-NHCH₂CONH)-1-methyl-1H-pyrrol-2-ylcarbonylamino]-1-methyl-1H-pyrrol-2-ylcarbonylamino]-1-methyl-1H-pyrrole-2-carboxylic Acid 8 (Following FIG. 1)

A solution of methyl 4-{4-[(Boc-NHCH₂CONH)-1-methyl-1H-pyrrol-2-yl-carbonylamino]-1-methyl-1H-pyrrol-2-ylcarbonylamino]-1-methyl-1H-pyrrole-2-carboxylate 7 (5.56 g, 10.0 mmole) in MeOH (100 ml), containing 5N NaOH (10 ml, 50 mmole), was stirred for 5 h at 60C and evaporated. The residue was dissolved in water (200 ml) and acidified with 1N HCl up to pH 3.5. The yellowish precipitate was collected, washed with water (5×10 ml) and dried in vacuo over phosphorus pentoxide to give 4-{4-[(Boc-NHCH₂CONH)-1-methyl-1H-pyrrol-2-ylcarbonylamino]-1-methyl-1H-pyrrol-2-ylcarbonyl-amino]-1-methyl-1H-pyrrole-2-carboxylic acid 8 was 4.98 g (92%).

¹H-NMR (DMSO-d₆): δ 9.93, 9.90 (s, s, 3H, —NHCH₂C(═O)NH-Py₁-, Py₁-C(═O)NH-Py₂-, Py₂-C(═O)NH-Py₃-); 7.40, 7.22, 7.14, 7.04, 6.90, 6.82 (d, d, d, d, d, d, 1H, 1H, 1H, 1H, 1H, 1H, H-3, H-5, Py₁, Py₂, Py₃); 6.99 (t, 1H, Boc-NHCH₂); 3.81, 3.80 (s, 9H, NCH₃, Py₁, Py₂, Py₃); 3.64 (d, 2H, Boc-NHCH₂); 1.37 (s, 9H, Boc).

### Example 9

### Preparation of Pentafluorophenyl 4-{4-[(Boc-NHCH₂CONH)-1-methyl-1H-pyrrol-2-yl-carbonylamino]-1-methyl-1H-pyrrol-2-ylcarbonylamino]-1-methyl- 1H-pyrrole-2-carboxylate 9 (Following FIGS. 1 and 3)

A solution of 4-{4-[(Boc-NHCH₂CONH)-1-methyl-1H-pyrrol-2-ylcarbonylamino]-1-methyl-1H-pyrrol-2-ylcarbonylamino]-1-methyl-1H-pyrrole-2-carboxylic 8 (5.3 g, 9.8 mmole), pentafluorophenol (2.03 g, 11.0 mmole) and DCC (2.27 g, 11.0 mmole) in DMF (50 ml), was stirred for 18 h at ambient temperature. DCU was filtered off and discarded. The filtrate was evaporated. The residue was chromatographed over a silica gel column (5.0×20 cm) using 50% AcOEt/toluene as eluent to give pentafluorophenyl 4-{4-[(Boc-NHCH₂CONH)-1-methyl-1H-pyrrol-2-yl-carbonylamino]-1-methyl-1H-pyrrol-2-yl-carbonylamino]-1-methyl-1H-pyrrole-2-carboxylate 9 5.25 g (84%) as a white foam.

¹H-NMR (DMSO-d₆): δ 10.06, 9.92, 9.77 (s, s, s, 1H, 1H, 1H, —NHCH₂C(═O)NH-Py₁-, Py₁-C(═O)NH-Py₂-, Py₂-C(═O)NH-Py₃-); 7.72, 7.28, 7.24, 7.14, 7.09, 6.90 (d, d, d, d, d, d, 1H, 1H, 1H, 1H, 1H, H-3, H-5, Py₁, Py₂, Py₃); 6.98 (t, 1H, Boc-NHCH₂); 3.88, 3.85, 3.83 (s, s, s, 3H, 3H, 3H, NCH₃, Py₁, Py₂, Py₃); 3.64 (d, 2H, Boc-NHCH₂); 1.38 (s, 9H, Boc).

¹⁹F-NMR (DMSO-d₆): δ −153.63 (m, 2F, F-2, F-6, -OPfp); −158.38 (m, 1F, F-4, -OPfp); −162.83 (m, 2F, F-3, F-5, -OPfp).

### Example 10

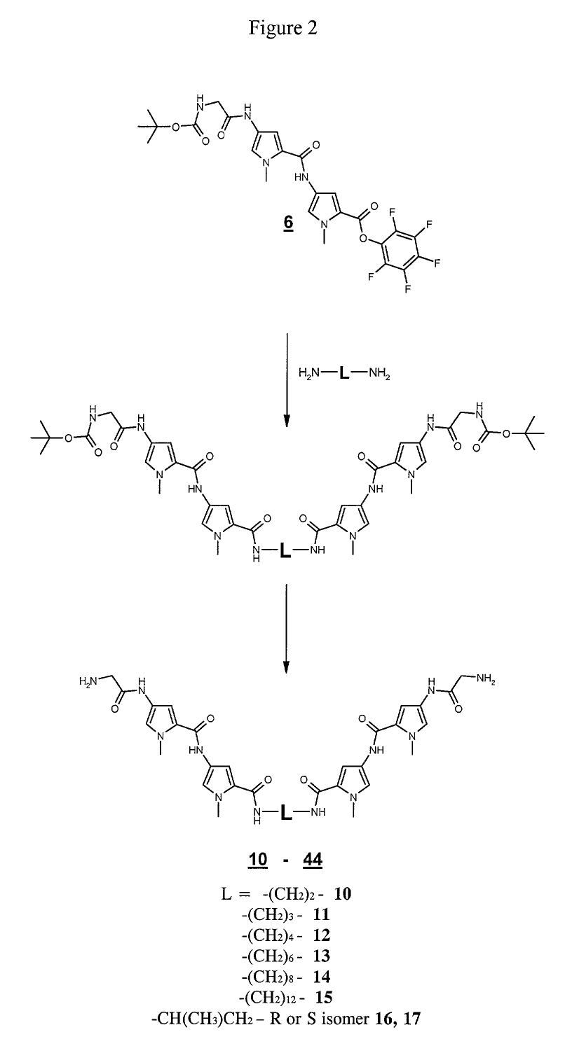

### Preparation of bis-1,2-{4-[(NH₂CH₂CONH)-1-methyl-1H-pyrrol-2-yl-carbonylamino]-1-methyl-1H-pyrrol-2-ylcarbonylamino}ethane 10 (Following FIG. 2)

Step 1

A solution of pentafluorophenyl 4-[4-(Boc-NHCH₂CONH)-1-methyl-1H-pyrrol-2-ylcarbonyl-amino]-1-methyl-1H-pyrrole-2-carboxylate 6 (129 mg, 0.22 mmole) and 1,2-ethylenediamine (60 mg, 0.10 mmole) in dry DMF (2.0 ml) was kept at ambient temperature for 72 hours and evaporated. The residue was chromatographed over a silica gel column (1.0×15 cm) using 5% MeOH/chloroform, containing 0.05% of 24% ammonium hydroxide, as eluent to give 73.3 mg (85%) of di-Boc-protected compound.

¹H-NMR (DMSO-d₆): δ 9.86, 9.80 (s, s, 2H, 2H, Boc-NHCH₂C(═O)NH-Py-, PyC(═O)NH-Py-); 8.08 (bs, 2H, PyC(═O)NHCH₂CH₂); 7.17, 7.13, 6.86, 6.84 (d, d, d, d, 2H, 2H, 2H, 2H, H-3, H-5, Py₁, Py₂); 6.99 (t, 2H, Boc-NHCH₂); 3.80, 3.79 (s, s, 6H, 6H, NCH₃, Py₁, Py₂); 3.63 (d, 4H, Boc-NHCH₂); 3.29 (s, 4H, PyC(═O)NHCH₂CH₂); 1.38 (s, 18H, Boc).

Step 2

A solution of di-Boc-protected compound (6.0 mg, 0.07 mmole) in 2M HCl MeOH/dioxane (1:1) (2.0 ml) was kept at ambient temperature for 30 min and evaporated. The residue was diluted with 0.1% TFA and purified by HPLC to give 34.3 mg (55%) of the title compound 10.

¹H-NMR (DMSO-d₆): δ 10.27, 9.83 (s, s, 2H, 2H, NH₂CH₂C(═O)NH-Py₁-, Py₁-C(═O)NH-Py₂); 8.02 (bs, 2H, PyC(═O)NHCH₂CH₂); 7.17, 7.15, 6.86, 6.85 (d, d, d, d, d, d, 2H, 2H, 2H, 2H, H-3, H-5, Py₁, Py₂); 3.83, 3.80 (s, s, 6H, 6H, NCH₃, Py₁, Py₂); 3.69 (s, 4H, NH₂CH₂); 3.31 (s, 4H, PyC(═O)NHCH₂CH₂).

### Example 11

### Preparation of bis-1,3-{4-[(NH₂CH₂CONH)-1-methyl-1H-pyrrol-2-yl-carbonylamino]-1-methyl-1H-pyrrol-2-yl-carbonylamino}propane 11 (Following FIG. 2)

Step 1

A solution of pentafluorophenyl 4-[4-(Boc-NHCH₂CONH)-1-methyl-1H-pyrrol-2-ylcarbonyl-amino]-1-methyl-1H-pyrrole-2-carboxylate 6 (129 mg, 0.22 mmole) and propanenediamine-1,3 (74 mg, 0.10 mmole) in dry DMF (2.0 ml) was kept at ambient temperature for 72 hours and evaporated. The residue was chromatographed over a silica gel column (1.0×15 cm) using 5% MeOH/chloroform, containing 0.05% of 24% ammonium hydroxide, as eluent to give 66.6 mg (76%) of di-Boc-protected compound.

¹H-NMR (DMSO-d₆): δ 9.87, 9.80 (s, s, 2H, 2H, Boc-NHCH₂C(═O)NH-Py-PyC(═O)NH-Py-); 8.03 (t, 2H, PyC(═O)NHCH₂—); 7.17, 7.13, 6.87, 6.84 (d, d, d, d, 2H, 2H, 2H, 2H, H-3, H-5, Py₁, Py₂); 6.99 (t, 2H, Boc-NHCH₂); 3.81, 3.79 (s, s, 6H, 6H, NCH₃, Py₁, Py₂); 3.64 (d, 4H, Boc-NHCH₂); 3.20 (m, 4H, —NHCH₂CH₂CH₂NH—); 1.66 (m, 2H, —NHCH₂CH₂CH₂NH—); 1.38 (s, 18H, Boc).

Step 2

A solution of di-Boc-protected compound (60 mg, 0.07 mmole) in 2M HCl MeOH/dioxane (1:1) (2.0 ml) was kept at ambient temperature for 30 min and evaporated. The residue was diluted with 0.1% TFA and purified by HPLC to give 40 mg (55%) of the title compound 11.

### Example 12

### Preparation of bis-1,4-{4-[(NH₂CH₂CONH)-1-methyl-1H-pyrrol-2-yl-carbonylamino]-1-methyl-1H-pyrrol-2-yl-carbonylamino}butane 12 (Following FIG. 2)

Step 1

A solution of pentafluorophenyl 4-[4-(Boc-NHCH₂CONH)-1-methyl-1H-pyrrol-2-ylcarbonyl-amino]-1-methyl-1H-pyrrole-2-carboxylate 6 (129 mg, 0.22 mmole) and butanediamine-1,4 (88 mg, 0.10 mmole) in dry DMF (2.0 ml) was kept at ambient temperature for 72 hours and evaporated. The residue was chromatographed over a silica gel column (1.0×15 cm) using 5% MeOH/chloroform, containing 0.05% of 24% ammonium hydroxide, as eluent to give 73 mg (25%) of di-Boc-protected compound.

¹H-NMR (DMSO-d₆): δ 9.84, 9.79 (s, s, 2H, 2H, Boc-NHCH₂C(═O)NH-Py-, PyC(═O)NH-Py-); 8.01 (t, 2H, PyC(═O)NHCH₂—); 7.15, 7.13, 6.87, 6.83 (d, d, d, d, 2H, 2H, 2H, 2H, H-3, H-5, Py₁, Py₂); 6.99 (t, 2H, Boc-NHCH₂); 3.81, 3.78 (s, s, 6H, 6H, NCH₃, Py₁, Py₂); 3.63 (d, 4H, Boc-NHCH₂); 3.17 (m, 4H, —NHCH₂CH₂CH₂CH₂NH—); 1.66 (m, 4H, —NHCH₂CH₂CH₂CH₂NH—); 1.38 (s, 18H, Boc).

### Step 2

A solution of di-Boc-protected compound (73 mg , 0.07 mmole) in 2M HCl MeOH/dioxane (1:1) (2.0 ml) was kept at ambient temperature for 30 min and evaporated. The residue was diluted with 0.1% TFA and purified by HPLC to give 35 mg (57%) of the title compound 12.

### Example 13

### Preparation of bis-1,6-{4-[(NH₂CH₂CONH)-1-methyl-1H-pyrrol-2-yl-carbonylamino]-1-methyl-1H-pyrrol-2-yl-carbonylamino}hexane 13 (Following FIG. 2)

Step 1

A solution of pentafluorophenyl 4-[4-(Boc-NHCH₂CONH)-1-methyl-1H-pyrrol-2-ylcarbonyl-amino]-1-methyl-1H-pyrrole-2-carboxylate 6 (129 mg, 0.22 mmole) and hexanediamine-1,4 (116 mg, 0.10 mmole) in dry DMF (2.0 ml) was kept at ambient temperature for 72 hours and evaporated. The residue was chromatographed over a silica gel column (1.0×15 cm) using 5% MeOH/chloroform, containing 0.05% of 24% ammonium hydroxide, as eluent to give 80 mg (87%) of di-Boc-protected compound.

¹H-NMR (DMSO-d₆): δ 9.84, 9.79 (s, s, 2H, 2H, Boc-NHCH₂C(═O)NH-Py-, PyC(═O)NH-Py-); 8.01 (t, 2H, PyC(═O)NHCH₂—); 7.15, 7.13, 6.87, 6.83 (d, d, d, d, 2H, 2H, 2H, 2H, H-3, H-5, Py₁, Py₂); 6.99 (t, 2H, Boc-NHCH₂); 3.81, 3.78 (s, s, 6H, 6H, NCH₃, Py₁, Py₂); 3.63 (d, 4H, Boc-NHCH₂); 3.17 (m, 4H, —NHCH₂CH₂CH₂CH₂NH—); 1.48 (m, 4H, —NHCH₂CH₂CH₂—); 1.38 (s, 18H, Boc); 1.48 (m, 4H, —NHCH₂CH₂CH₂—).

Step 2

A solution of di-Boc-protected compound (60 mg, 0.065 mmole) in 2M HCl MeOH/dioxane (1:1) (2.0 ml) was kept at ambient temperature for 30 min and evaporated. The residue was diluted with 0.1% TFA and purified by HPLC to give 35 mg (57%) of the title compound 13.

### Example 14

### Preparation of bis-1,8-{4-[(NH₂CH₂CONH)-1-methyl-1H-pyrrol-2-yl-carbonylamino]-1-methyl-1H-pyrrol-2-yl-carbonylamino}octane 14 (Following FIG. 2)

Step 1

A solution of pentafluorophenyl 4-[4-(Boc-NHCH₂CONH)-1-methyl-1H-pyrrol-2-ylcarbonyl-amino]-1-methyl-1H-pyrrole-2-carboxylate 6 (129 mg, 0.22 mmole) and 1,8-octanediamine (134 mg, 0.10 mmole)in dry DMF (2.0 ml) was kept at ambient temperature for 72 hours and evaporated. The residue was chromatographed over a silica gel column (1.0×15 cm) using 5% MeOH/chloroform, containing 0.05% of 24% ammonium hydroxide, as eluent to give 85 mg (90%) of di-Boc-protected compound.

¹H-NMR (DMSO-d₆): δ 9.84, 9.79 (s, s, 2H, 2H, Boc-NHCH₂C(═O)NH-Py-, PyC(═O)NH-Py-); 7.96 (t, 2H, PyC(═O)NHCH₂—); 7.15, 7.13, 6.87, 6.81 (d, d, d, d, 2H, 2H, 2H, 2H, H-3, H-5, Py₁, Py₂); 6.99 (t, 2H, Boc-NHCH₂); 3.81, 3.77 (s, s, 6H, 6H, NCH₃, Py₁, Py₂); 3.63 (d, 4H, Boc-NHCH₂); 3.13 (m, 4H, —NHCH₂CH₂CH₂CH₂NH—); 1.46 (m, 4H, —NHCH₂CH₂CH₂CH₂—); 1.38 (s, 18H, Boc); 1.27 (m, 8H, —NHCH₂CH₂CH₂CH₂—).

Step 2

A solution of di-Boc-protected compound (70.0 mg, 0.074 mmole) in 2M HCl MeOH/dioxane (1:1) (2.0 ml) was kept at ambient temperature for 30 min and evaporated. The residue was diluted with 0.1% TFA and purified by HPLC to give 44 mg (61%) of the title compound 14.

### Example 15

### Preparation of bis-1, 10-{4-[(NH₂CH₂CONH)-1-methyl-1H-pyrrol-2-yl-carbonylamino]-1-methyl-1H-pyrrol-2-yl-carbonylamino}dodecane 15 (Following FIG. 2)

Step 1

A solution of pentafluorophenyl 4-[4-(Boc-NHCH₂CONH)-1-methyl-1H-pyrrol-2-ylcarbonyl-amino]-1-methyl-1H-pyrrole-2-carboxylate 6 (129 mg, 0.22 mmole) and 1,12-dodecanediamine (134 mg, 0.10 mmole) in dry DMF (2.0 ml) was kept at ambient temperature for 72 hours and evaporated. The residue was chromatographed over a silica gel column (1.0×15 cm) using 5% MeOH/chloroform, containing 0.05% of 24% ammonium hydroxide, as eluent to give 81 mg (81%) of di-Boc-protected compound.

¹H-NMR (DMSO-d₆): δ 9.85, 9.81 (s, s, 2H, 2H, Boc-NHCH₂C(═O)NH-Py-, PyC(═O)NH-Py-); 7.97 (t, 2H, PyC(═O)NHCH₂—); 7.17, 7.15, 6.89, 6.82 (d, d, d, d, 2H, 2H; 2H, 2H, H-3, H-5, Py₁, Py₂); 7.01 (t, 2H, Boc-NHCH₂); 3.82, 3.78 (s, s, 6H, 6H, NCH₃, Py₁, Py₂); 3.65 (d, 4H, Boc-NHCH₂); 3.14 (m, 4H, —NHCH₂CH₂CH₂CH₂CH₂CH₂—); 1.46 (m, 4H, —NHCH₂CH₂CH₂CH₂—); 1.38 (s, 18H, Boc); 1.26 (bs, 16H, —NHCH₂CH₂CH₂CH₂CH₂CH₂—).

Step 2

A solution of di-Boc-protected compound (60 mg, 0.06 mmole) in 2M HCl MeOH/dioxane (1:1) (2.0 ml) was kept at ambient temperature for 30 min and evaporated. The residue was diluted with 0.1% TFA and purified by HPLC to give 37 mg (61%) of the title compound 15.

### Example 16

### Preparation of 1-(R)-bis-1,2-{4-[(NH₂CH₂CONH)-1-methyl-1H-pyrrol-2-yl-carbonylamino]-1-methyl-1H-pyrrol-2-yl-carbonylamino} -1-methylethane 16 (Following FIG. 2)

Step 1

A solution of pentafluorophenyl 4-[4-(Boc-NHCH₂CONH)-1-methyl-1H-pyrrol-2-ylcarbonyl-amino]-1-methyl-1H-pyrrole-2-carboxylate 6 (129 mg, 0.22 mmole) and (R)-(+)-propanediamine-1,2 (74 mg, 0.10 mmole)in dry DMF (2.0 ml) was kept at ambient temperature for 72 hours and evaporated. The residue was chromatographed over a silica gel column (1.0×15 cm) using 5% MeOH/chloroform, containing 0.05% of 24% ammonium hydroxide, as eluent to give 64 mg (74%) of di-Boc-protected compound.

¹H-NMR (DMSO-d₆): δ 9.85, 9.84, 9.76 (s, s, s, 4H, Boc-NHCH₂C(═O)NH-Py-, PyC(═O)NH-Py-); 8.08 (t, 1H, PyC(═O)NHCH₂—); 7.83 (d, 1H, —CH(CH₃)NH-Py-); 7.17, 7.13, 6.89, 6.86, 6.86 (d, s, d, s, d, 8H, H-3, H-5, Py₁, Py₂); 6.98 (t, 2H, Boc-NHCH₂); 4.09 (m, 1H, —CH(CH₃)NH-Py-); 3.81, 3.80, 3.79, 3.77 (s, s, s, s, 3H, 3H, 3H, 3H, NCH₃, Py₁, Py₂); 3.65 (d, 4H, Boc-NHCH₂); 3.32 (m, 2H, -PyC(═O)NHCH₂—); 1.38 (s, 18H, Boc); 1.10 (d, 3H, —CH(CH₃)NH-Py-).

Step 2

A solution of di-Boc-protected compound (50.0 mg, 0.06 mmole) in 2M HCl MeOH/dioxane (1:1) (2.0 ml) was kept at ambient temperature for 30 min and evaporated. The residue was diluted with 0.1% TFA and purified by HPLC to give 33 mg (65%) of the title compound 16.

### Example 17

### Preparation of 1-(S)-bis-1,2-{4-[(NH₂CH₂CONH)-1-methyl-1H-pyrrol-2-yl-carbonylamino]-1-methyl-1H-pyrrol-2-yl-carbonylamino}-1-methylethane 17 (Following FIG. 2)

Step 1

A solution of pentafluorophenyl 4-[4-(Boc-NHCH₂CONH)-1-methyl-1H-pyrrol-2-ylcarbonyl-amino]-1-methyl-1H-pyrrole-2-carboxylate 6 (129 mg, 0.22 mmole) and (S)-(−)-propanediamine-1,2 (74 mg, 0.10 mmole)in dry DMF (2.0 ml) was kept at ambient temperature for 72 hours and evaporated. The residue was chromatographed over a silica gel column (1.0×15 cm) using 5% MeOH/chloroform, containing 0.05% of 24% ammonium hydroxide, as eluent to give 68 mg (79%) of di-Boc-protected compound.

¹H-NMR (DMSO-d₆): δ 9.88, 9.86, 9.82 (s, s, s, 4H, Boc-NHCH₂C(═O)NH-Py-, PyC(═O)NH-Py-); 8.11 (t, 1H, PyC(═O)NHCH₂—); 7.86 (d, 1H, —CH(CH₃)NH-Py-); 7.20, 7.15, 6.91, 6.88, 6.86 (d, s, d, s, d, 8H, H-3, H-5, Py₁, Py₂); 7.01 (t, 2H, Boc-NHCH₂); 4.10 (m, 1H, —CH(CH₃)NH-Py-); 3.83, 3.82, 3.81, 3.79 (s, s, s, s, 3H, 3H, 3H, 3H, NCH₃, Py₁, Py₂); 3.65 (d, 4H, Boc-NHCH₂); 3.30 (m, 2H, -PyC(═O)NHCH₂—); 1.39 (s, 18H, Boc); 1.12 (d, 3H, —CH(CH₃)NH-Py-).

Step 2

A solution of di-Boc-protected compound (60 mg, 0.07 mmole) in 2M HCl MeOH/dioxane (1:1) (2.0 ml) was kept at ambient temperature for 30 min and evaporated. The residue was diluted with 0.1% TFA and purified by HPLC to give 40 mg (55%) of the title compound 17.

Proceeding as described in Example 10 above, using compound 6 but substituting the 1,2-ethylenediamine with the diamines listed in Table I below, provided the corresponding compound of Formula (I) 18-44.

TABLE I
Compounds synthesized according to Table (I)
(Compounds 18-44)

| FIG. | | | |
|---|---|---|---|
| II | | | Calculated |
| Cpd | | ES-MS: | for |
| # | Diamine | found | (M + H) |
| 18 | 1,2-hexadecanediamine | 859.54 | 859.53 |
| 19 | 1,3-cyclohexane(bismethylamine) | 745.39 | 745.39 |
| 20 | 1,4-cyclohexane(bismethylamine) | 745.33 | 745.39 |
| 21 | 4,4′-methylenebis(cyclohexylamine) | | 813.45 |
| 22 | L-lysinamide | 748.35 | 748.36 |
| 23 | 2,7-diaminofluorene | 844.38 | 844.31 |
| | | | M + 2Na |
| 24 | m-xylenediamine | 739.36 | 739.34 |
| 25 | p-xylenediamine | 739.35 | 739.34 |
| 26 | meso-1,2-Diphenylethylenediamine | 815.38 | 815.37 |
| 27 | L-lysine β-naphtylamide | 438.2 | 874.41 |
| | | M/2 + H | |
| 28 | L-lysine p-nitroanilide | 869.36 | 869.38 |
| 29 | L-lysine pentafluorophenylhydrazide | 465.17 | 929.36 |
| | | M/2 + H | |
| 30 | L-lysine (4-trifluoromethylpyrimidin-2-yl) | 455.18 | 909.38 |
| | hydrazide | M/2 + H | |
| 31 | L-lysine 2-(pyrene-1-yl)ethylamide | 488.72 | 976.46 |
| | | M/2 + H | |
| 32 | L-lysine 2-(pyrene-1-yl)butylamide | 502.72 | 1003.48 |
| | | M/2 + H | |
| 33 | L-lysine α-naphtylamide | 438.2 | 874.41 |
| | | M/2 + H | |
| 34 | L-lysine 2-(4-nitrophenyl)ethylamide | 897.39 | 897.41 |
| 35 | L-lysine 2-(6-nitrobenzimidazol-1-yl)- | 469.12 | 937.42 |
| | ethylamide | M/2 + H | |
| 36 | L-lysine 2-(indol-3-yl)ethylamide | 891.42 | 891.44 |
| 37 | L-lysine 2-(5-fluoroindol-3-yl)-ethylamide | 909.41 | 909.43 |
| 38 | L-lysine carbobenzoxyhydrazide | | 897.41 |
| 39 | L-diaminopropionic acid | | 854.36 |
| | carbobenzoxyhydrazide | | |
| 40 | L-lysine (pyrene-1-yl)acetylhydrazide | 503.20 | 1005.45 |
| | | M/2 + H | |
| 41 | L-lysine 2-(pyrene-1-yl)methylamide | 962.50 | 962.44 |
| 42 | Bis (3-aminopropyl)-carbamic acid benzyl | 868.46 | 868.42 |
| | ester | | |
| 43 | Bis (3-aminopropyl)-methylamine | 748.35 | 748.40 |
| 44 | 1,4-Bis (3-aminopropyl)-piperazine | 803.44 | 803.44 |

### Example 18

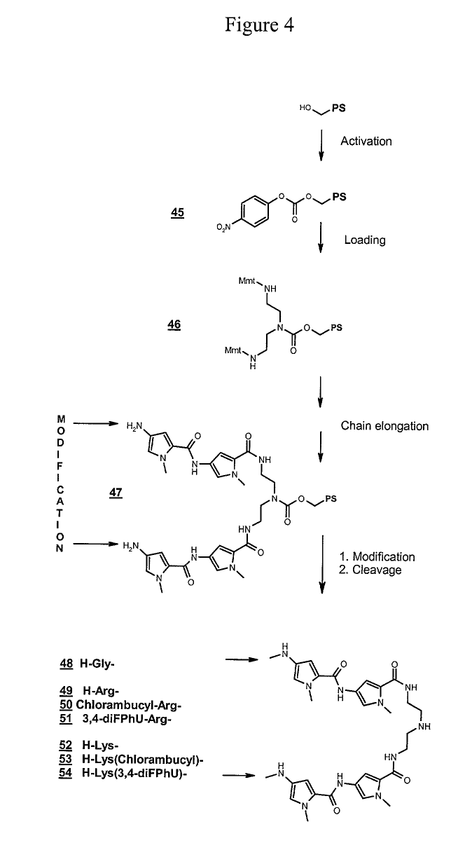

### Preparation of N,N-bis-{4-[(NH₂CH₂CONH)-1-methyl-1H-pyrrol-2-yl-carbonylamino]-1-methyl-1H-pyrrol-2-yl-carbonylaminoethyl}amine 48 (Following FIG. 4)

Step 1

Hydroxymethyl polystyrene (HMPS) resin (2.0 g, S=0.67 mmol/g) was swelled in DCM (25 mL) for 10 minutes. 4-Nitrophenylchloroformate (0.54 g, 2.68 mmol) was added followed by NMM (0.295 mL, 2.68 mmol). The mixture was agitated overnight (18 hrs) at room temperature. The next morning the resin was drained on a fritted funnel, was washed six times with DCM, 3 times with ether and was dried in high vacuum (4 hrs). As calculated from the weight of the activated dry resin 45 (2.25 g), the activation was quantitative.

Step 2

Activated resin 45 (0.8 g, 0.54 mmol) was swelled in DMF (8 mL). Bis-[monomethoxytritylaminoethyl]amine (0.39 g, 0.6 mmol) was added followed by DMAP (0.15 g, 1.2 mmol). The mixture was agitated for two days at room temperature. Methylamine (10 mL, 2M) in THF was added and the quenching reaction proceeded for two hours. The resin was then drained, washed twice with DMF, three times with 10% DIEA in DMF, three times with DMF, three times with DCM, twice with MeOH and twice with ether. It was then dried under high vacuum to yield loaded resin 46 (0.97 g). The degree of substitution was determined by picric acid titration of the amino groups being present after a 10-minute-treatment of a resin aliquot with 50% TFA in DCM followed by thorough washing with DCM and neutralization with 10% DIEA in DMF. The substitution, S=0.38 mmol/g (i.e. Samino=0.76 mmol/g) was high enough for the synthesis of polyamides.

Step 3

Resin bound compound 46 (0.97 g, 0.76 mmol protected amine) was swelled in DCM for 10 minutes. The resin was then drained and treated twice with TFA (20 mL 50%), 2% anisol in DCM for 1 and 20 minutes, respectively. The amino resin was washed three times with DCM and three times with DMF. Meanwhile, in a separate vial, Boc-Py-OH (0.58 g, 2.4 mmol) was dissolved in DMF (2 mL) and was pre-activated by the addition of HBTU (0.865 g, 2.28 mmol), HOBt (0.325 g, 2.4 mmol) and DIEA (0.83 mL, 4.8 mmol). The pre-activation proceeded for 2 minutes at room temperature. The activated acid was added to the drained amino-resin and the mixture was agitated for 1 hr at room temperature. The completeness of the reaction was checked with the Kaiser test described in Kaiser, E. et al., Anal. Biochem., 71, 261, (1970). The resin was drained, washed three times with DMF and three times with DCM.

Step 4

To incorporate the second pyrrole units, the above cycle was repeated the same way to get the bis-Boc protected compound 47 which was deblocked through a double treatment with the 50% TFA/2% anisol/DCM solution for 1 and 20 minutes, respectively. The free amino containing compound 47 was washed three times with DCM, three times with MeOH and twice with ether. The resin was divided at this stage into parts to make the diversifying modifications separately.

Step 5

In a separate vessel, Boc-Gly-OH (53 mg, 0.3 mmol) was dissolved in DMF (2 mL) and was treated with HBTU (108 mg, 0.28 mmol), HOBt (40 mg, 0.3 mmol) and DIEA (104 μL, 0.6 mmol) at room temperature for 2 min. 1/7^{th} of the above resin (140 mg, about 0.1 mmol amine) was reacted with the activated Boc-Gly-OH for 1 hr at room temperature followed by draining, washing with DMF (3×), DCM (3×), MeOH (2×) and ether (2×). The resin bound N,N-{4-[(NH₂CH₂CONH)-1-methyl-1H-pyrrol-2-yl-carbonylamino]-1-methyl-1H-pyrrol-2-yl-carbonylaminoethyl}amine was dried to give 146 mg dry material.

Step 6

To the resin bound N,N-{4-[(NH₂CH₂CONH)-1-methyl-1H-pyrrol-2-yl-carbonyl-amino]-1-methyl-1H-pyrrol-2-yl-carbonylaminoethyl}amine (146 mg, 0.1 mmol), thioanisol (250 mL), ethanedithiol (125 mL) and TFA (2.5 mL) were added. The mixture was cooled to 0° C. and TFMSA (300 μL) was added drop wise with continuous stirring. The reaction vessel was sealed, and was shaken for 2 hrs at room temperature. The resin was filtered off, washed with pure TFA (1 mL) and the product was precipitated from the supernatant with 40 mL cold ether. The precipitate was spinned down, the supernatant was discarded. The precipitate was washed two more times with ether then was dried. The product was purified with HPLC (Vydac 12 μm C₁₈ 2.2×25 cm column, 0% to 60% acetonitrile gradient over 30 minutes, flow 20 mL/min). The overall yield of the title compound 48 was (36 mg, 52%).

ES MS: 706.37 (calcd. for M+H⁺: 706.35).

Proceeding as described in Example 18 above, using compound 47 but substituting Boc-Gly with Boc-Arg(Mts)-OH, compound 49 was formed after removing both protecting groups. To make compounds 50 and 51, compound 47 was first coupled with Boc-Arg(Mts)-OH, then partial deblocking (removal of Boc) followed by coupling with chlorambucyl or 3,4-difluorophenyl isocyanate and removal of Mts group produced compounds 50 and 51, respectively.

Proceeding as described in Example 18 above, using compound 47 but substituting Boc-Gly with Boc-Lys(Fmoc)-OH, compound 52 was formed after removing both protecting groups. To make compounds 53 and 54, compound 47 was first coupled with Boc-Lys(Fmoc)-OH, then partial deblocking (removal of Fmoc) followed by coupling with chlorambucyl or 3,4-difluorophenyl isocyanate and removal of Boc group produced compounds 53 and 54, respectively.

TABLE II
Compounds synthesized according to Table (I)
(Compounds 49-54)

| CPD # | | ES-MS: | Calcd. for |
|---|---|---|---|
| in FIG. IV | Amino acid | found | (M + H) |
| 49 | Arginine | 903.51 | 903.51 |
| 50 | Chlorambucylarginine | 1476.59 | 1476.61 |
| 51 | 3,4-diFPhU-arginine | 1214.51 | 1214.55 |
| 52 | Lysine | 848.50 | 848.49 |
| 53 | Lysine(chlorambucyl) | 1420.65 | 1420.59 |
| 54 | Lysine(3,4-diFPhU) | 1158.49 | 1158.54 |

### Example 19

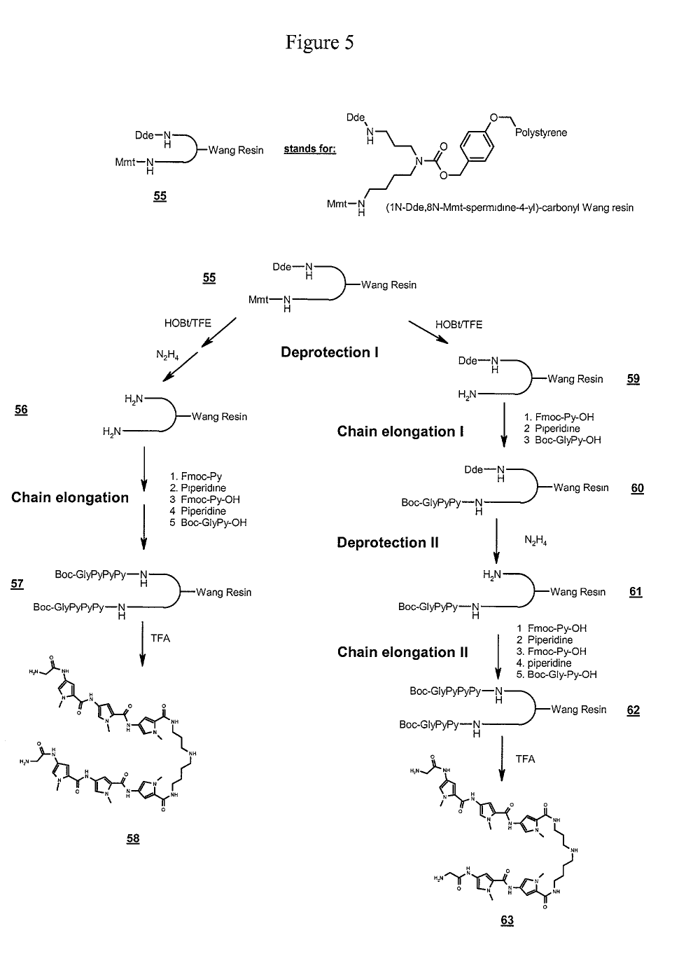

### Preparation of N,N-bis-{4-{4-[(NH₂CH₂CONH)-1-methyl-1H-pyrrol-2-yl-carbonylamino]-1-methyl-1H-pyrrol-2-ylcarbonylamino}-1-methyl-1H-pyrrol-2-yl-carbonylaminopropyl}amine 58 (Following FIG. 5)

Step 1

(1N-Dde, 8N-Mmt-Spermidine-4-yl-carbonyl-Wang resin 55 (100 mg, 0.04 mmol, S=0.42 mmol/g) was swelled in DCM for 5 minutes and then treated with 1M HOBt (2.5 mL) in TFE/DCM 1:1 for 1 hr at room temperature. The resin was drained, washed with DCM (2x), DMF (2×). Hydrazine hydrate (2.5 μL, 2%) in DMF was added and the mixture was agitated for 5 minutes. The hydrazine treatment was repeated three more times without washing the resin between the treatments. After the fourth treatment the resin was washed with DMF (6×) to give compound 56.

Step 2

Fmoc-Py-OH (86.9 mg, 0.24 mmol) was dissolved in DMF (2 mL) and was activated by addition of HATU (86.6 mg, 0.22 mmol), HOAt (16.3 mg, 0.24 mmol) and DIEA (83 μL, 0.48 mmol) for 2 minutes at room temperature. The activated acid was poured into the drained amino-resin and was reacted for 2 hrs at room temperature. The resin was drained, washed with DMF (4×) then was treated with 20% piperidine in DMF twice for 1 and 20 minutes, respectively, followed by washing with DMF (6×) to complete the first elongation cycle.

The cycle was repeated in the same way first using Fmoc-Py-OH (86.9 mg, 0.24 mmol) again then using Boc-Gly-Py-OH (71.4 mg, 0.24 mmol). The latter coupling should have been repeated to make the reaction complete. The resin was washed with DMF (3×), DCM (3×), MeOH (2×), ether (2×) then was dried to give 96 mg resin bound polyamide 57.

Step 3

The product was cleaved from the resin by treatment with 95% TFA (5 mL), 2.5% EDT, 2.5% water for 1 hr at room temperature. The resin was then filtered off, washed with 1 mL pure TFA and the product was precipitated from the supernatant with cold ether (40 mL). The precipitate was spun down, and the supernatant was discarded. The precipitate was washed two more times with ether then was dried. The product was purified with HPLC (Vydac 12 μm C₁₈ 2.2×25 cm column, 0% to 60% acetonitrile gradient over 30 minutes, flow 20 mL/min). The overall yield of the title compound 58 was (1.2 mg, 3%). ES\MS: 992.59 (calcd. for M+H⁺: 994.51).

### Example 20

### Preparation of N-{4-{4-[(NH₂CH₂CONH)-1-methyl-1H-pyrrol-2-yl-carbonylamino]-1-methyl-1H-pyrrol-2-ylcarbonylamino}-1-methyl-1H-pyrrol-2-yl-carbonylamino-propyl}-N-{4-[(NH₂CH₂CONH)-1-methyl-1H-pyrrol-2-yl-carbonylamino]-1-methyl-1H-pyrrol-2-yl-carbonylaminopropyl}amine 63 (Following FIG. 5)

Step 1

1N-Dde, 8N-Mmt-Spermidine-4-yl0-carbonyl-Wang resin, 55 (S=0.42 mmol/g, 100 mg, 0.04 mmol) was swelled in DCM for 5 minutes then was treated with 1M HOBt (2.5 mL) in TFE/DCM 1:1 for 1 hr at room temperature. The resin was drained, washed with DCM (3×), DMF (3×) to get the half protected diamino-resin, 59.

Step 2

Fmoc-Py-OH (86.9 mg, 0.24 mmol) was dissolved in DMF (2 mL) and was activated by addition of HATU (86.6 mg, 0.22 mmol), HOAt (16.3 mg, 0.24 mmol) and DIEA (83 μL, 0.48 mmol) for 2 minutes at room temperature. The activated acid was poured into the drained amino-resin and was reacted for 2 hrs at room temperature. The resin was drained, washed with DMF (4×) then was treated with 20% piperidine in DMF twice for 1 and 20 minutes, respectively, followed by washing with DMF (6×) to complete the first elongation cycle.

In the second cycle the procedure was repeated twice using Boc-Gly-Py-OH (71. 4 mg, 0.24 mmole) each time, to give compound 60.

Step 3

Before continuing the synthesis on the shorter arm of the spermidine linker the Dde protection was removed by treating the resin four times for 5 minutes with 2% hydrazine hydrate (2.5 mL) in DMF each time. The resin was then washed with DMF (6×) to give compound 61.

Step 4

Fmoc-Py-OH (86.9 mg, 0.24 mmol) was dissolved in DMF (2 mL) and was activated by addition of HATU (86.6 mg, 0.22 mmol), HOAt (16.3 mg, 0.24 mmol) and DIEA (83 μL, 0.48 mmol) for 2 minutes at room temperature. The activated acid was poured into the drained amino-resin and was reacted for 2 hrs at room temperature. The resin was drained, washed with DMF (4×) then was treated with 20% piperidine in DMF twice for 1 and 20 minutes, respectively, followed by washing with DMF (6×) to complete the third elongation cycle.

The cycle was repeated in the same way first using Fmoc-Py-OH (86.9 mg, 0.24 mmol) again then using Boc-Gly-Py-OH (71.4 mg, 0.24 mmol). The latter coupling step was performed two times to make the reaction complete. The resin was washed with DMF (3×), DCM (3×), MeOH (2×), ether (2×) then was dried to give 96 mg resin bound polyamide, compound 62.

Step 5

The title compound 63 was cleaved from the resin by treatment with 95% TFA (5 mL), 2.5% EDT, 2.5% water for 1 hr at room temperature. The resin was then filtered off, washed with pure TFA (1 mL) and the product was precipitated from the supernatant with 40 mL cold ether. The precipitate was spun down, and the supernatant was discarded. The precipitate was washed two more times with ether then was dried. The product was purified with HPLC (Vydac 12 μm C₁₈ 2.2×25 cm column, 0% to 60% acetonitrile gradient over 30 minutes, flow 20 mL/min). The overall yield of the title compound 63 was (2.6 mg, 7.5%).

ES MS: 872.39 (calcd. for M+H⁺: 872.46).

### Example 21

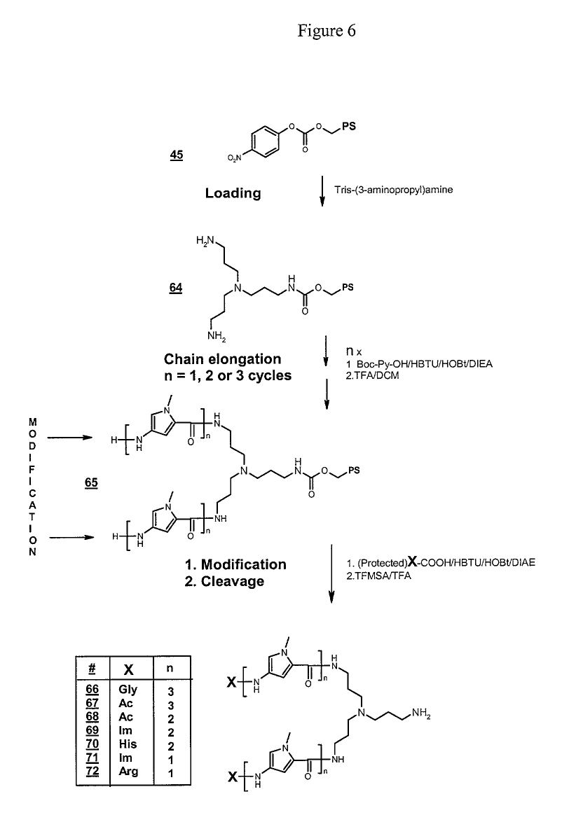

### Preparation of N,N-bis-{4-{4-[(NH₂CH₂CONH)-1-methyl-1H-pyrrol-2-yl-carbonylamino]-1-methyl-1H-pyrrol-2-ylcarbonylamino}-1-methyl-1H-pyrrol-2-yl-carbonylaminopropyl}-N-(3-aminoethylpropyl)amine 66 (Following FIG. 6)

Step 1

Activated resin 45 (2.58 g, 2.58 mmol) was swelled in DMF (15 mL). Tris-(3-aminopropyl)amine (2.5 mL, 12.5 mmol) was added followed by DMAP(0.61 g, 5 mmol). The mixture was agitated overnight at room temperature. The resin was then drained, washed twice with DMF, three times with 10% DIEA in DMF, three times with DMF, three times with DCM, twice with MeOH and twice with ether. It was then dried under high vacuum to yield 2.84 g of loaded resin 64. The degree of substitution was determined by picric acid titration, S=0.26 mmol/g (i.e. Sₐₘᵢₙₒ=0.52 mmol/g).

Step 2

The synthesis of the poly-pyrrole part was carried out as described in Example 18 for compound 47 starting with compound 64 (150 mg, 0.04 mmol), except the initial TFA treatment was omitted and three synthesis cycles were done to give 185 mg resin pound polyamide compound 65.

Step 3

Boc-Gly-OH (53 mg, 0.3 mmol) was dissolved in DMF (2 mL) and was treated with HBTU (108 mg, 0.28 mmol), 40 mg HOBt (0.3 mmol) and 104 μL (0.6 mmol) DIEA at room temperature for 2 min. Compound 65 (185 mg, 0.08 mmol amine) was reacted with the activated Boc-Gly-OH for 1 hr at room temperature followed by draining, washing with DMF (3×), DCM (3×), MeOH (2×) and ether (2×). The resin bound product was dried before cleavage to give 190 mg dry material.

Step 4

To the polyamide resin (190 mg, 0.04 mmol), thioanisol (250 μL), ethanedithiol (125 μL) and TFA (2.5 mL) were added. The mixture was cooled to 0° C. and TFMSA (300 μL) was added dropwise with continuous stirring. The reaction vessel was sealed, and was shaken for 2 hrs at room temperature. The resin was filtered off, washed with pure TFA (1 mL) and the product was precipitated from the supernatant with 40 mL cold ether. The precipitate was spun down, and the supernatant was discarded. The precipitate was washed two more times with ether then was dried. The product was purified with HPLC (Vydac 12 μm C₁₈ 2.2×25 cm column, 0% to 60% acetonitrile gradient over 30 minutes, flow 20 mL/min). The overall yield of the title compound was 4.5 mg (11%).

ES MS: 1035.62 (calcd. for M+H⁺: 1035.54).

Proceeding as described above, but substituting glycine with acetic anhydride, Im-OH, Boc-Arg(Mts)-OH or Boc-His(Bom)-OH, respectively provided compounds 66-72 shown in Table III below.

TABLE III
Compounds synthesized according to FIG. VI
(compounds 66-72)

| | | | ES-MS: | Calculated for |
|---|---|---|---|---|
| CPD # | X | n | found | (M + H) |
| 67 | Ac | 3 | 1005.60 | 1005.52 |
| 68 | Ac | 3 | 761.49 | 761.42 |
| 69 | Im | 2 | 893.56 | 893.47 |
| 70 | His | 2 | 951.67 | 951.52 |
| 71 | Im | 1 | 649.43 | 649.38 |
| 72 | Arg | 1 | 745.70 | 745.51 |

### Example 22

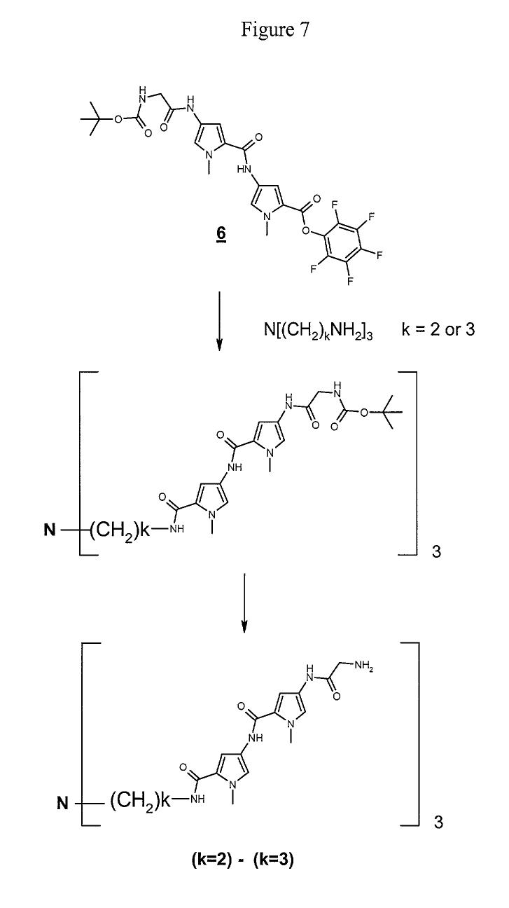

### Preparation of tris-{4-[(NH₂CH₂CONH)-1-methyl-1H-pyrrol-2-yl-carbonylamino]-1-methyl-1H-pyrrol-2-yl-carbonylaminoethyl}amine 73 (Following FIG. 7)

Compound 73 was synthesized as described for Compound 10 above.

### Example 23

### Preparation of tris-{4-[(NH₂CH₂CONH)-1-methyl-1H-pyrrol-2-yl-carbonylamino]-1-methyl-1H-pyrrol-2-yl-carbonylaminopropyl}amine 74 (Following FIG. 7)

Compound 74 was synthesized as described for Compound 10 above.

### Example 24

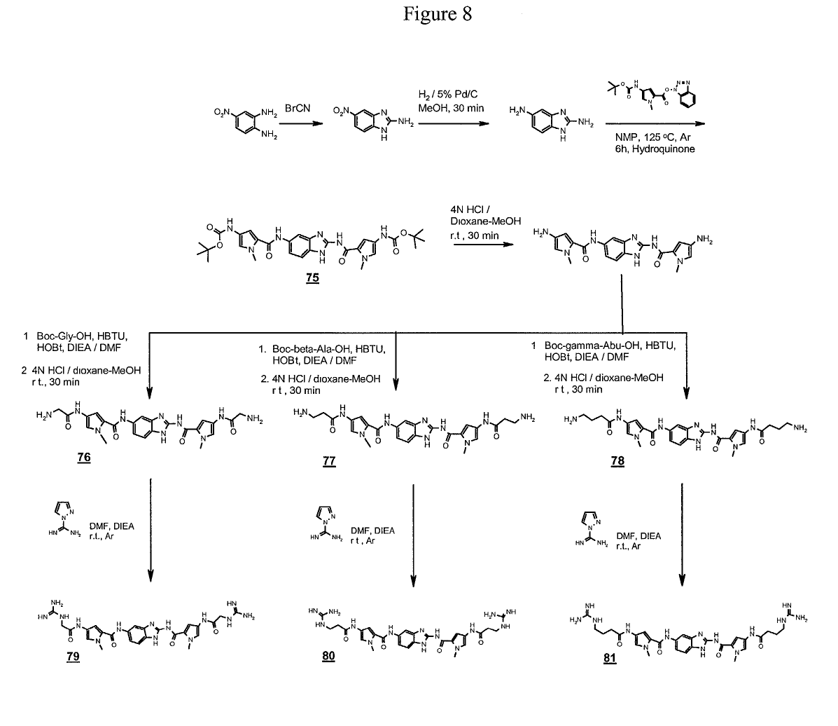

### Preparation of 1,5-bis-{4-[(NH₂CH₂CONH)-1-methyl-1H-pyrrol-2-yl-carbonylamino]-benzimidazole 76 (Following FIG. 8)

Step 1

4-Nitro-1,2-phenylenediamine (2.76 g, 17.66 mmol) was suspended in MeOH (200 ml) and 5 M BrCN (4 ml) in MeCN was added dropwise in a period of 20 min followed by 50 ml of water. The resulting reaction mixture was stirred at room temperature overnight and 30 ml of water was added. The reaction mixture was concentrated to about 80 ml and washed with AcOEt (50 ml×3). The combined AcOEt phase was extracted with 40 ml of water and then discarded. The combined water phase was made basic with a saturated NaHCO₃ solution. The yellow precipitate formed was filtered, washed with cold water and dried. The filtrate was extracted with AcOEt (50 ml×2). The combined AcOEt phase was washed with brine (25 ml), dried over anhydrous Na₂SO₄, and evaporated to give 2-amino-4-nitrobenzimidazole (2.94 g, 93%) as a yellow solid. ¹HNMR (DMSO-d₆): δ 7.92 (s, H), 7.84 (1H, d, J=7.8 Hz), 7.16 (1H, d, J=8.1 Hz), 6.88 (s, 2H); MS: 179.00 (M+1).

Step 2

2-Amino-4-nitrobenzimidazole (0.211 g, 1.18 mmol) in MeOH (20 ml) was hydrogenated under 35 psi of H₂ over 5% palladium on activated carbon for 30 min. After removal of Pd/C, methanol was evaporated to give a colorless solid 2,5-diamino-benzimidazole. A mixture of above 2,5-diaminobenzimidazole, Boc-Py-OBt (0.868 g, 2.43 mmol) and a trace of hydroquinone (1 mg) in NMP (8 ml) was stirred in the dark under argon at 125° C. for 6 h and then cooled to RT. The product was diluted with hexane/ether (2:1) and the precipitate was centrifuged. Further purification was performed by column chromatography using CHCl₃—MeOH (7:1) as eluent to give 1,5-bis-[(4-(tert-butoxycarbonylamino)-1-methyl-1H-pyrrol-2-yl-carbonylamino]benzimidazole 75 (0.38 g, 54%) as a pale yellow powder. ¹H-NMR (DMSO-d₆): δ 9.67 (s, 1H), 9.08 (s, 1H), 9.04 (s, 1H), 7.81 (s, 1H), 7.31-7.24 (m, 2H), 7.03-6.99 (m, 2H), 6.85 (d, 2H, J=5.7 Hz), 3.83 (s, 3H), 3.75 (s, 3H), 1.40 (s, 18H); MS: 593.27 (M+1).

Step 3

A solution of 1,5-bis-[4-(tert-butoxycarbonylamino)-1-methyl-1H-pyrrol-2-yl-carbonylamino]benzimidazole 75 (68 mg, 0.115 mmol) in 2 M HCl in MeOH-dioxane (1:1, 6 ml) was stirred at ambient temperature for 30 min and evaporated to yield 1,5-bis-(4-amino-1-methyl-1H-pyrrol-2-yl-carbonylamino)benzimidazole MS: 393.17 (M+1), 197.07 (M/2+1).

Step 4

A solution of Boc-Gly-OH (40.4 mg, 0.231 mmol), HOBt (87.4 mg, 0.231 mmol), and HBTU (35.4 mg, 0.231 mmol) and DIEA (80 μl ) in anhydrous DMF (5 ml) was stirred at ambient temperature for 4 min., and then transferred to a stirred solution of 1,5-bis-(4-amino-1-methyl-1H-pyrrol-2-yl-carbonylamino)benzimidazole and DIEA (80 μl)in of anhydrous DMF (2 ml). The reaction mixture was stirred at ambient temperature overnight and evaporated. The resulted solid was purified by column chromatography using CHCl₃—MeOH (6:1) as eluent to give 1,5-bis-[4-(BocNHCH₂CONH)-1-methyl-1H-pyrrol-2-yl-carbonylamino)benzimidazole (74.4 mg, 92%) as a pale brown solid. MS: 707.37 (M+1).

Step 5

The solution of purified 1,5-bis-[4-(BocNHCH₂CONH)-1-methyl-1H-pyrrol-2-yl-carbonylamino)benzimidazole (18 mg) in 2 M HCl in MeOH-dioxane (1:1, 4 ml) was stirred at ambient temperature for 30 min and evaporated. The resulted compound was purified by reverse phase HPLC to yield 1,5-bis-[4-(NH₂CH₂CONH)-1-methyl-1H-pyrrol-2-yl-carbonylamino)benzimidazole 14.8 mg (79%) of compound 76. MS: 507.24 (M+1), 254.13 (M/2+1).

### Example 25

### Preparation of 1,5-bis-{4-[(NH₂(CH₂)₂CONH)-1-methyl-1H-pyrrol-2-yl-carbonylamino]-benzimidazole 77 (Following FIG. 8)

Compound 77 was synthesized as described in Example 24, starting from compound 75 (86.6 mg, 0.146 mmol) and Boc-β-aminopropionic acid (55.5 mg, 0.293 mmol). Yield of compound 77: 19.4 mg (81%). ¹H-NMR (D₂O) δ 7.50 (d, 1H), 7.15 (d, 1H, J=9 Hz), 7.02 (m, 1H, J=1.5, 9.0 Hz), 6.95 (d, 1H), 6.84 (d, 1H, J=1.5 Hz), 6.75 (d, 1H, J=1.5 Hz), 6.47 (s, 1H), 3.62 (s, 3H), 3.54 (s, 3H), 3.36-3.30 (m, 4H), 2.47-2.40 (m, 4H). MS: 535.26 (M+1), 268.14 (M/2+1).

### Example 26

### Preparation of 1,5-bis-{4-[(NH₂(CH₂)₃CONH)-1-methyl-1H-pyrrol-2-yl-carbonylamino]-benzimidazole 78 (Following FIG. 8)

Compound 78 was synthesized as described in Example 24, starting from compound 75 (81.4 mg, 0.137 mmol) and Boc-Υ-aminobutyric acid (57.8 mg, 0.275 mmol). Yield of compound 78: 22.5 mg (86%). MS (ESI) 563.37 (M+1), 282.19 (M/2+1).

### Example 27

### Preparation of 1,5-bis-{4-[(guanidino(CH₂)CONH)-1-methyl-1H-pyrrol-2-yl-carbonylamino]benzimidazole 79 (Following FIG. 8)

A solution of compound 76 (36.2 mg, 0.0493 mmol), 1H-pyrazole-1-carboxamidine hydrochloride (14.5 mg, 0.0986 mmol) and DIEA (43 μl) in DMF (3 ml) was stirred under argon at 45° C. overnight and evaporated to dryness. The product was purified by reverse phase HPLC to yield 4.6 mg (11%) of the title compound 79. MS: 591.27 (M+1), 296.14 (M/2+1).

### Example 28

### Preparation of 1,5-bis-{4-[(guanidino(CH₂)₂CONH)-1-methyl-1H-pyrrol-2-yl-carbonylamino]benzimidazole 80 (Following FIG. 8)

Compound 77 (36.9 mg, 0.0493 mmol), 1H-pyrazole-1-carboxamidine hydrochloride (14.5 mg, 0.0986 mmol) and DIEA (43 μl ) in DMF (3 ml) was stirred under argon at 45° C. overnight and evaporated to dryness. The product was purified by reverse phase HPLC to yield 7.8 mg (16%) the title compound 80. MS: 310.15 (M/2+1), 207.10 (M/3+1).

### Example 29

### Preparation of 1,5-bis-{4-[(guanidino(CH₂)₃CONH)-1-methyl-1H-pyrrol-2-yl-carbonylamino]-benzimidazole 81 (Following FIG. 8)

Compound 78 (37.6 mg, 0.0493 mmol), 1H-pyrazole-1-carboxamidine hydrochloride (14.5 mg, 0.0986 mmol) and DIEA (43 μl ) in DMF (3 ml) was stirred under argon at 45° C. overnight and evaporated to dryness. The product was purified by reverse phase HPLC to yield 4.5 mg (14%) the title compound 81. MS: 324.18 (M/2+1), 216.45 (M/3+1).

### Example 30

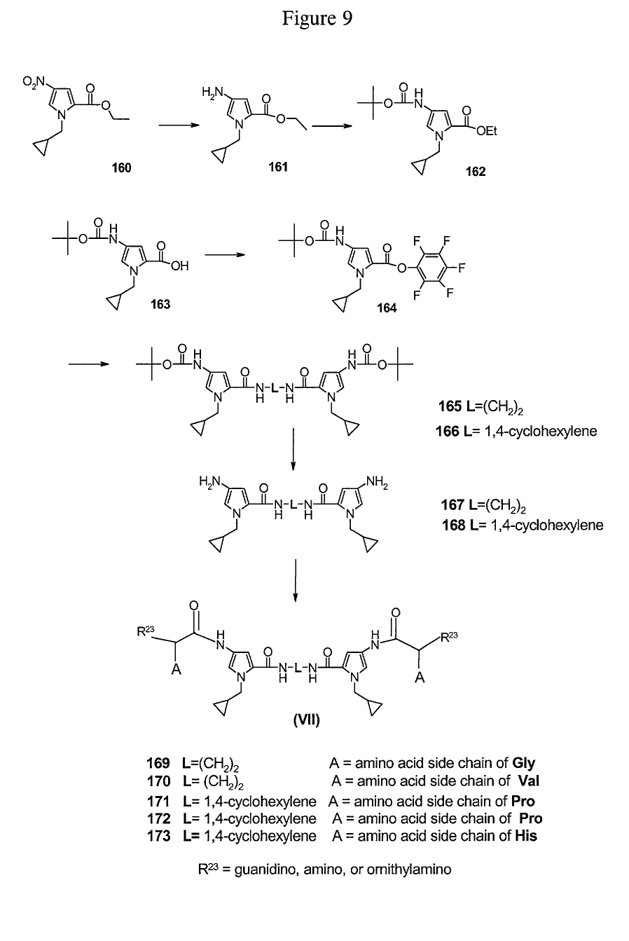

### Bis-1,2-[4-(2-amino-acetylamino)-1-cyclopropylmethyl-1H-pyrrol-2-yl-carbonylamino]-ethane 169. (Following FIG. 9)

Step A: Synthesis: 4-tert-Butoxycarbonylamino-1-cyclopropylmethyl-1H-pyrrole-2-carboxylic Acid 163

To a stirred solution of 1-Cyclopropylmethyl-4-nitro-1H-pyrrole-2-carboxylic acid ethyl ester 160 (256 mg, 1 mmol) in methanol (50 ml) was added 10% Pd/C (Degussa type, Aldrich) (0.5 g). The flask was evacuated and then flushed 3 times with hydrogen and finally filled with hydrogen at 25-30 psi. The resultant suspension was stirred vigorously at 23° C. for 45 min. The suspended material was filtered and the filtrate was evaporated to dryness. The resulting 1-Cyclopropylmethyl-4-amino-1H-pyrrole-2-carboxylic acid ethyl ester 161 was used for the next step without purification. Aminopyrrole 161 was dissolved in 50 ml of DMF and di-tert-butyl-dicarbonate (300 mg) was added. The reaction mixture was allowed to stay at ambient temperature overnight and evaporated. The residue was dissolved in ethyl acetate (50 ml). The organic solution was washed with 10% citric acid (2×10 ml), brine (10 ml), saturated solution of sodium bicarbonate (2×10 ml) and brine again (10 ml). Ethyl acetate solution was dried over sodium sulfate and evaporated. The 4-tert-Butoxycarbonylamino-1-cyclopropylmethyl-1H-pyrrole-2-carboxylic acid ethyl ester 162 was suspended in 50 ml of 2N NaOH and stirred at 55° C. until a clear solution was obtained (3 hours). Then 1N HCl was added to the reaction mixture until the pH was 2. The white precipitate was filtered, washed with water and dried to yield 600 mg (70%) of 163.

H¹-NMR (DMSO-d₆): δ 0.37-0.42, 0.65-0.72 (m, 2H, CH₂), 1.22-1.28 (m, 1H, CH), 1.57 (s, 9H, CH₃), 4.23 (d, 2H, CH₂), 7.44 and 7.81 (d, 1H, pyrrole), 8.85 (s, 1H, NHCO). MS 279.56 (M−1).

Step B: 4-tert-Butoxycarbonylamino-1-cyclopropylmethyl-1H-pyrrole-2-carboxylic Acid Pentafluorophenyl Ester 164.

Pyrrole carboxylic acid 163 (600 mg, 2.14 mmol) was dissolved in the mixture of 50 ml of dry DMF and diisopropyl ethylamine (552 μl, 3 mmol). Pentafluorophenol trifluoroacetate (531 μl, 3 mmol) was added and the reaction mixture was kept for 2 hours at ambient temperature. The solvent was evaporated and the residue purified by column chromatography on silica gel in toluene to yield 800 mg (83%) of title compound.

H¹-NMR (DMSO-d₆): δ 0.37-0.42, 0.65-0.72 (m, 2H, CH₂), 1.22-1.28 (m, 1H, CH), 1.57 (s, 9H, CH₃), 4.23 (d, 2H, CH₂), 6.70 and 7.17 (s, 1H, pyrrole), 9.03 (s, 1H, NHCO). F¹⁹-NMR (DMSO-d₆): δ −45964 (t), −44719 (t), −43380 (d).

Step C: Bis-1,2-(4-tert-Butoxycarbonylamino-1-cyclopropylmethyl-1H-pyrrol-2-yl-carbonylamino)-ethane 165.

Ethylenediamine (48 mg, 0.8 mmol) was added to the solution of 164 (800 mg, 1.8 mmol) in 10 ml of dry DMF and left overnight at 60° C. The DMF was evaporated and the residue was purified by column chromatography on silica gel in chloroform/methanol 97:3 to yield 500 mg of trimer 165 (91%).

H¹-NMR (DMSO-d₆): δ 0.37-0.42, 0.65-0.72 (m, 2H, CH₂), 1.22-1.28 (m, 1H, CH), 1.57 (s, 9H, CH₃), 3.02 (s, 2H, ethylene), 4.23 (d, 2H, CH₂), 6.39 and 6.66 (s, 1H, pyrrole), 7.78 and 8.80 (s, 1H, NHCO).

Step D: Bis-1,2-[4-(2-amino-acetylamino)-1-cyclopropylmethyl-1H-pyrrol-2-yl-carbonylamino]-ethane 169.

Trimer 165 (0.5 mmol, 292 mg) was dissolved in 10 ml trifluoroacetic acid/ anisole/methylene chloride (3/2/5 v/v/v) and in 30 min evaporated. Compound 167 was coevaporated with dry DMF and dissolved in 25 ml of dry DMF. BocGly (175 mg, 1 mmol) was dissolved in 5 ml of DMF, 1-hydroxybenzotriazole (135 mg, 1 mmol), O-benzotriazol-1-yl-N,N,N′,N′-tetramethyluronium hexafluorophosphate (379 mg, 1 mmol) and DIEA (400 μl) were added. The mixture was kept at room temperature for 2 min and added to 167. In 10 hours the solvent was evaporated, the residue was distributed between water and chloroform. The water was extracted with chloroform (2×15 ml). The organic fractions were washed with water, dried over sodium sulfate and evaporated. The Boc-protection was removed as described above for 165. The title compound 169 was isolated by HPLC. Yield 60% of compound 169. MS 499.68 (M+H⁺).

### Example 31

### Bis-1,2-[4-(2-amino-3-methyl-butyrylamino)-1-cyclopropylmethyl-1H-pyrrol-2-yl-carbonylamino]-ethane 170. (Following FIG. 9)

Compound 170 was synthesized as described for compound 169 above (example 1). Activated Boc-valine was used on step D to treat aminotrimer 167. Yield 56% of compound 171. ES MS: 583.73. (M+H⁺).

### Example 32

### Bis-trans-1,4-[4-(pyrrolidine-2-carbonyl-amino)-1-cyclopropylmethyl-1H-pyrrol-2-yl-carbonylamino]cyclohexane 171. (Following FIG. 9)

Step A: Bis-trans-1,4-(4-tert-Butoxycarbonylamino -1-cyclopropylmethyl-1H-pyrrol-2-yl-carbonylamino)-cyclohexane 166.

1,4-trans-diaminocyclohexane (205 mg, 1.8 mmol) was added to the solution of 164 (892 mg, 2 mmol) in 10 ml of dry DMF and left overnight at 60° C. DMF was evaporated and the residue was purified by column chromatography on silica gel in chloroform/methanol 97:3 to yield 500 mg of trimer 166 (80%).

H¹-NMR (DMSO-d₆): δ 0.37-0.42, 0.65-0.72 (m, 2H, CH₂ ), 1.22-1.28 (m, 1H, CH), 1.09-1.19 (m, 2H, CH₂, cyclohexane), 1.27 (s, 9H, CH₃), 1.45-1.60 (m, 2H, CH₂, cyclohexane), 3.27 (m, 1H, CH cyclohexane), 6.39 and 6.66 (s, 1H, pyrrole), 7.78 and 8.80 (s, 1H, NHCO).

Step B: Bis-trans-1,4-[4-(pyrrolidine-2-carbonyl-amino)-1-cyclopropylmethyl-1H-pyrrol-2-yl-carbonylamino]cyclohexane 171.

Trimer 166 (0.14 mmol, 90 mg) was dissolved in 10 ml trifluoroacetic acid/anisole/methylene chloride (3/2/5 v/v/v) and in 30 min evaporated. The obtained 168 was coevaporated with dry DMF and dissolved in 25 ml of dry DMF. BocPro (65 mg, 0.3 mmol) was dissolved in 5 ml of DMF, 1-hydroxybenzotriazole (41 mg, 3 mmol), O-benzotriazol-1-yl-N,N,N′,N′-tetramethyluronium hexafluorophosphate (113 mg, 0.3 mmol) and DIEA (110 μl) were added. The mixture was kept at room temperature for 2 min and added to 168. In 10 hours the solvent was evaporated, the residue was distributed between water and chloroform. The water was extracted with chloroform (2×15 ml). The organic fractions were washed with water, dried over sodium sulfate and evaporated. The Boc-protection was removed as described above for 165. The title compound 171 was isolated by HPLC. Yield 68% of compound 169. MS 633.61 (M+H⁺).

### Example 33

### Bis-trans-1,4-{[4-(2,5-diamino-pentanoyl)pyrrolidine-2-carbonyl-amino]-1-cyclopropylmethyl-1H-pyrrol-2-yl-carbonylamino}cyclohexane 172. (Following FIG. 9)

Polyamide 171 as dichlorohydrate (42 mg, 0.06 mmol) was dissolved in 5 ml of dry DMF. DIEA was added followed with FmocOrn(Boc)OPfp (90 mg, 0.5 mmol). The mixture was kept at room temperature for 10 hours and solvent was evaporated. The residue was treated with 10% solution of piperidine in methylene chloride for 30 min. The solvent was evaporated, the residue dissolved in 5 ml of methanol and precipitated with 30 ml of ether. The precipitate was collected and dissolved in 10 ml trifluoroacetic acid/anisole/methylene chloride (3/2/5 v/v/v) and in 30 min evaporated. The residue dissolved in 5 ml of methanol and precipitated with 30 ml of ether. The precipitate was collected and isolated by HPLC. Yield 75% of compound 172. MS 861.41 (M+H⁺).

### Example 34

### Bis-trans-1,4-{4-[2-amino-3-(1H-imidazol-4-yl)propyonylamino]-1-cyclopropylmethyl-1H-pyrrol-2-yl-carbonylamino]cyclohexane 173. (Following FIG. 9)

Compound 173 was synthesized from compound 168 using FmocHis(Trt) as described in the example 3. Deblocking and isolation was done as described above for 172 in the Example 4. MS 713.99 (M+H⁺).

### Example 35

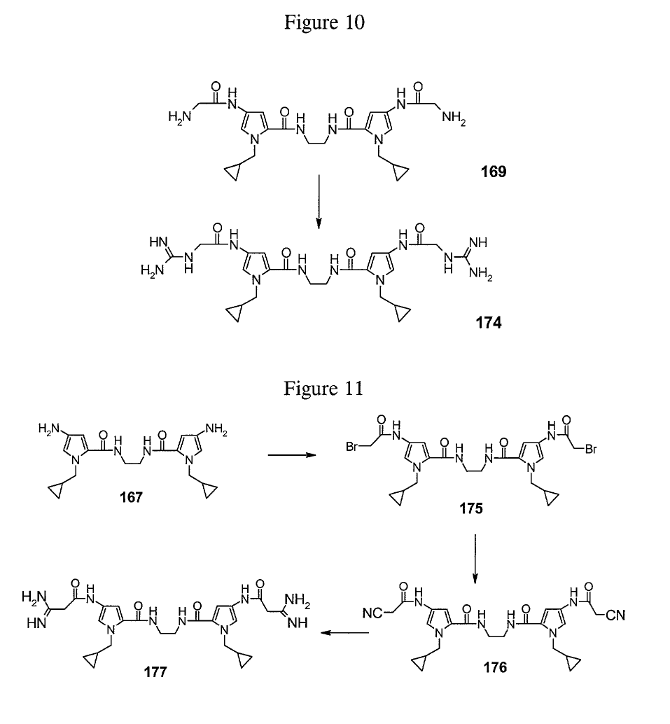

### Bis-1,2-[4-(2-guanidino-ethylamino)-1-cyclopropylmethyl-1H-pyrrol-2-yl-carbonylamino]-ethane 174. (Following FIG. 10)

TFA-salt of amine 169 (25 mg, 0.05 mmol) was dissolved in DMF (3 ml ), pyrazole-1-carboxamidine (13.2 mg, 0.12 mmol) was added and the reaction was stirred at ambient temperature overnight. The title product 174 was isolated by HPLC. The yield is 65%. MS 583.27 (M+H⁺).

### Example 36

### Bis-1,2-[4-(2-carbamimidoyl-acetylamino)-1-methyl-1H-pyrrol-2-yl-carbonylamino]-ethane 177 (Following FIG. 11)

Step A Bis-1,2-[4-(2-bromo-acetylamino)-1-methyl-1H-pyrrol-2-yl-carbonylamino]-ethane 175.

Diamine 167 (152 mg, 0.5 mmol) as TFA salt was prepared as described in example 1, step D. This amine was dissolved in 5 ml of dry methanol and 5 ml of 4N HCl/dioxane was added. The solvent was evaporated and diamine hydrochloride was used for former transformations. The dry residue was dissolved in the mixture of 15 ml of DMF and 400 μl of DIEA, bromoacetic acid (208 mg, 1,5 mmol) and EDCI (380 mg, 2 mmol) was added and the reaction mixture was kept overnight at ambient temperature. Water (50 ml) and ethyl acetate (50 ml) were added. The organic fraction was separated; the water fraction was extracted with ethyl acetate (6×20 ml). The combined organic fraction were washed with brine, dried over Na₂SO₄ and evaporated. The residue was crystallized from methanol/ether to yield 234 mg of 175 (86%).

H¹-NMR (DMSO-d₆): δ 3.29 (s, 2H, CH₂, ethylene), 3.78 (s, 3H, CH₃), 4.15 (s, 2H, CH₂, bromoacetyl), 6.71 and 7.14 (d, 1H, pyrrole), 8.12 and 10.20 (s, 1H, NHCO).

Step B Bis-1,2-[4-(2-cyano-acetylamino)-1-methyl-1H-pyrrol-2-yl-carbonylamino]-ethane 176.

Dibromide 175 (200 mg, 0.36 mmol) was dissolved in 20 ml of DMF, KCN (82 mg, 1.46 mmol) was added and the reaction was stirred overnight at 55° C. The product 176 was isolated by HPLC with the yield of 97 mg (55%).

H¹-NMR (DMSO-d₆): δ 3.26 (s, 2H, CH₂, ethylene), 3.76 (s, 3H, CH₃), 3.86(s, 2H, CH₂, cyanoacetyl), 6.72 and 7.11 (d, 1H, pyrrole), 8.22 and 10.68 (s, 1H, NHCO).

Step D Bis-1,2-[4-(2-carbamimidoyl-acetylamino)-1-methyl-1H-pyrrol-2-yl-carbonylamino]-ethane 177

A solution of cyano-derivative 176 (80 mg, 0.18 mmol) in 10 ml of dry ethanol was cooled to 0-5° C. and saturated with HCl gas. The mixture was sealed and refrigerated for 20 hours. The mixture was allowed to warm to room temperature and ethanol was evaporated. The resulting imino ester was dissolved in 10 ml of anhydrous ethanol and saturated with ammonia gas to get amidine 177. The title compound was isolated by HPLC. MS 237.12 (M+2H)²⁺.

### Example 37

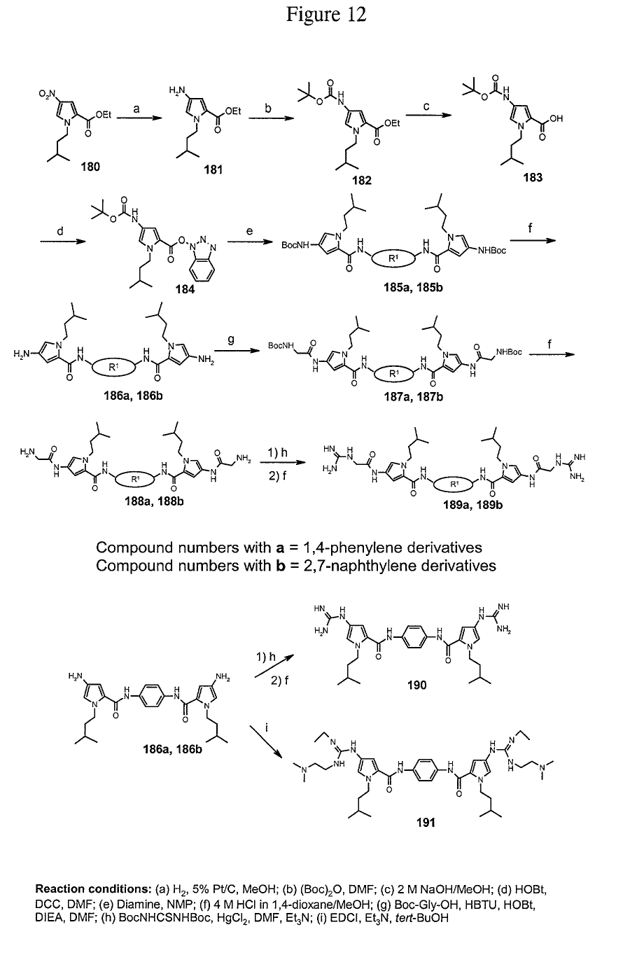

In FIG. 12 the synthesis of compounds of Formula (III) is shown. The synthesis is started using ethyl 1-isoamyl-4-nitropyrrole-2-carboxylate (180), which can be obtained from alkylation of ethyl 4-nitropyrrole-2-carboxlate with bromoisoamyl. Compound 180 is reduced to its amine 181 by hydrogenation over 5% Pd/C in methanol followed protection of Boc-group to give compound 182. After hydrolysis of 3 with 2M NaOH in Methanol, the corresponding acid 183 was obtained, which was coupled with 1-hydroxybenzotrizole (HOBt) in the presence of DCC in DMF to afford the activated ester 184. This ester was then coupled with 1,4-diaminobenzene or 2,7-diaminonaphthalene, which were obtained from the reduction of 4-nitroaniline or 2,7-dinitronaphathalene by catalytic hydrogenation over 5% Pd/C in methanol, to yield a 1,4-disubstituted phenylene derivative 185a or 2,7-disubstituted naphthalene derivative 185b, respectively. Deprotection of Boc-group with 4M HCl in 1,4-dioxane in methanol gave the corresponding amine 186a. The corresponding hydrochloride salt for 186a was used in the next step of the reaction. Compound 186a was coupled with Boc-glycine in the presence of HOBt and HBTU in DMF to afford their glycine derivatives 187a. Under the same reaction conditions used for the deprotection of the Boc-group with 4 M HCl, the corresponding amine 188a was obtained. The amine was converted to the guanidino derivatives 189a by treatment with Boc-protected thiourea and HgCl₂ in the presence of Et₃N in DMF, followed deprotection of Boc-group with 4 M HCl. The guanidino derivative, 189b was made using the same series of reactions, starting with 2,7-disubstituted naphthalene derivative 185b. Guanidination of compound 186a or 186b with Boc-protected thiourea or EDCI provided the corresponding guanidino 190 or 191.

### Example 38

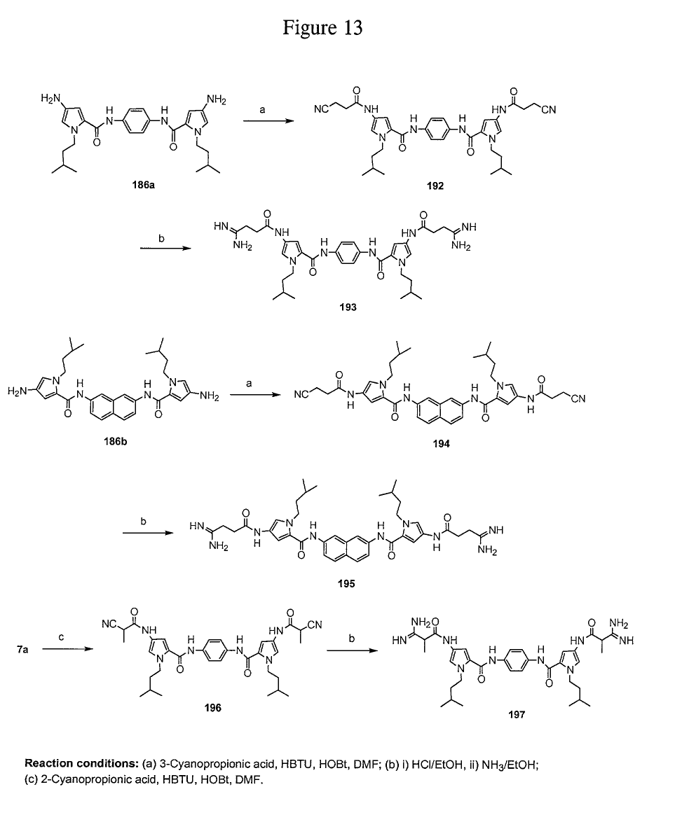

FIG. 13 shows a synthetic route for compounds of the general formula (IV). Compound 193 can be prepared first by coupling 186a with 3-cyanopropionic acid, which was from the hydrolysis of commercial 3-cyanopropinic acid methyl ester, to give compound 192. Pinner reaction on 192 with HCl in ethanol, followed by ammonia in ethanol provide amidino derivative 193. Under similar reaction conditions, compound 195 could be obtained from compound 186b via the preparation of compound 194. Condensation of 186a with 2-cyanopropinic acid in the presence of HBTU, HOBt, and Et₃N afforded its intermediate 196. This compound was consequently converted to the amide derivative 197 according to Pinner reaction.

### Example 39

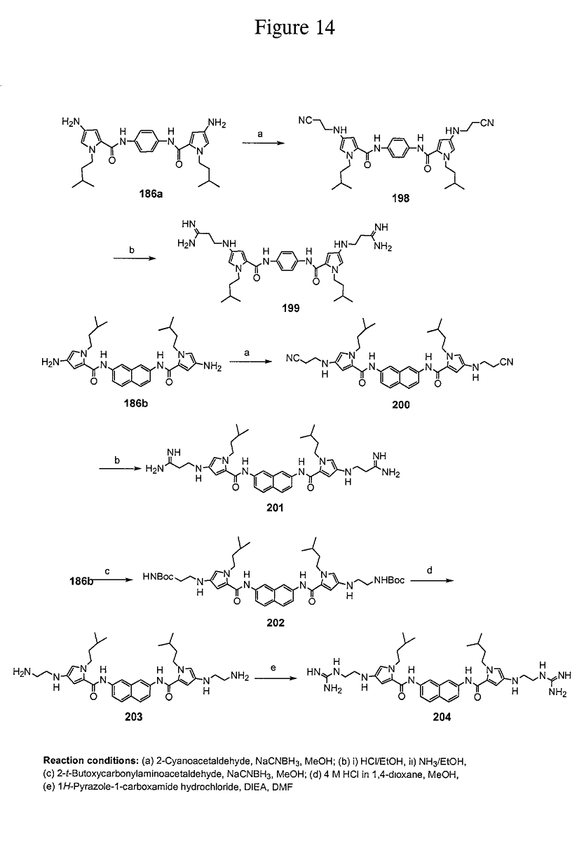

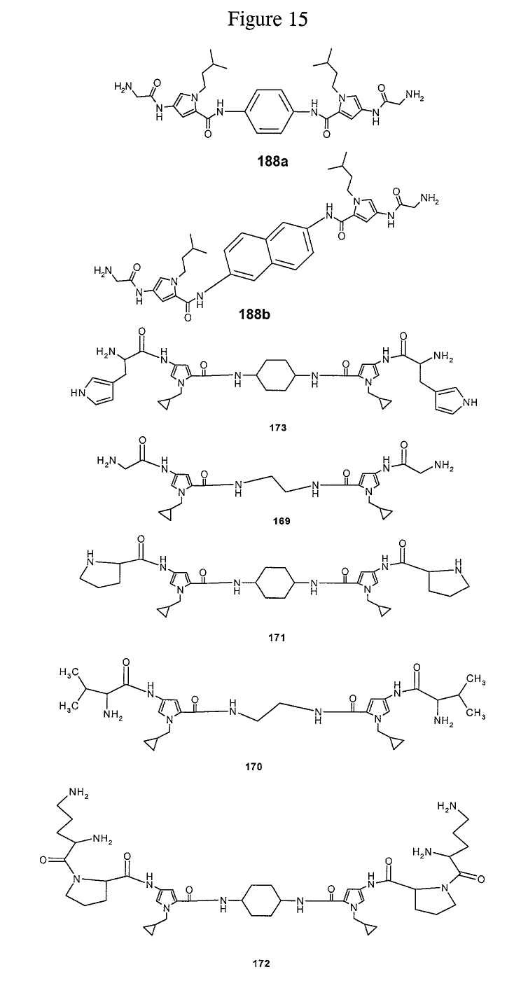

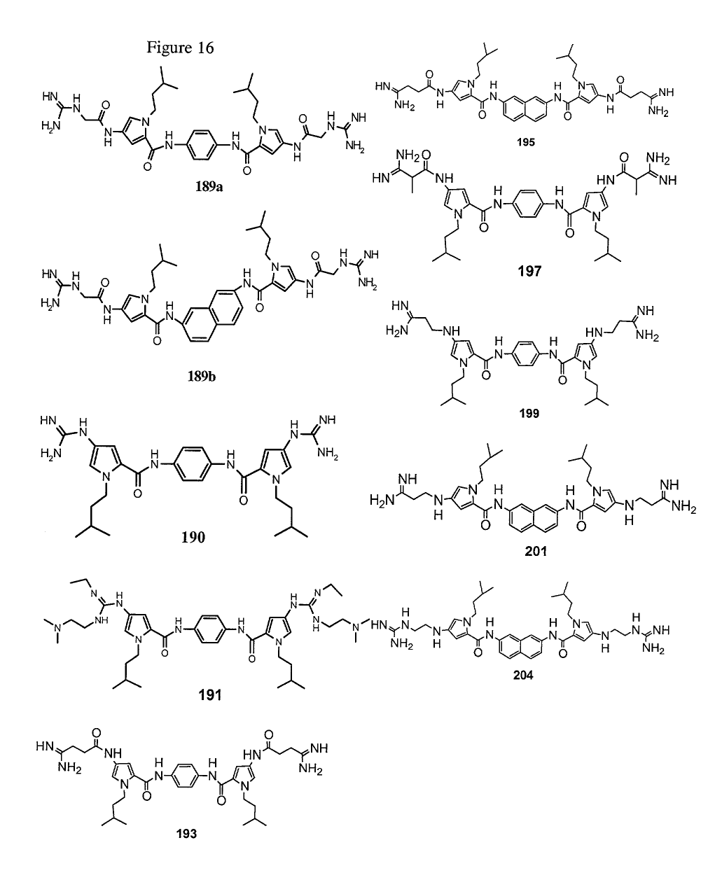

The preparation of compounds from formula (V) and (VI) are shown in FIG. 14. Amidation of compound 186a was achieved using 2-cyanoacetaldehyde in the presence of sodium cyanoboronhydride in methanol to give compound 198. Treatment of 198 with HCl in ethanol, followed by ammonia gave its amidine 199. Under above reaction conditions, compound 201 could be obtained from 186b via the preparation of compound 200. Amidation of compound 186b with 2-tert-butoxycarbonylaminoacetaldehyde provided Boc-protecting compound 202. Deprotection of Boc-group gave the amine 203, was then converted to the guanidine derivative 204 using 1H-pyrazole-1-carboxamidine hydrochloride in the presence of diisopropylethylamine in DMF.

### FORMULATION EXAMPLES

The following are representative pharmaceutical formulations containing a compound of Formula (I).

Example 1
Tablet formulation
The following ingredients are mixed intimately and pressed into
single scored tablets.

| Quantity per Ingredient | tablet, mg |
|---|---|
| compound of this invention | 400 |
| cornstarch | 50 |
| croscarmellose sodium | 25 |
| lactose | 120 |
| magnesium stearate | 5 |

Example 2
Capsule formulation
The following ingredients are mixed intimately and loaded into a
hard-shell gelatin capsule.

| | Quantity |
|---|---|
| Ingredient | per capsule, mg |
| compound of this invention | 200 |
| lactose, spray-dried | 148 |
| magnesium stearate | 2 |

Example 3
Suspension formulation
The following ingredients are mixed to form a suspension
for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | ml |
| colorings | 0.5 | mg |
| distilled water | q.s. to 100 | ml |

Example 4
Injectable formulation
The following ingredients are mixed to form an injectable formulation.

| Ingredient | Amount |
|---|---|
| compound of this invention | 0.2 mg-20 mg |
| sodium acetate buffer solution, 0.4 M | 2.0 ml |
| HCl (1N) or NaOH (1N) | q.s. to suitable pH |
| water (distilled, sterile) | q.s. to 20 ml |

Example 5
Suppository formulation
A suppository of total weight 2.5 g is prepared by mixing the
compound of the invention with Witepsol ® H-15 (triglycerides
of saturated vegetable fatty acid; Riches-Nelson, Inc., New York), and
has the following composition:

| Ingredient | Amount |
|---|---|
| compound of the invention | 500 mg |
| Witepsol ® H-15 | balance |

### BIOLOGICAL EXAMPLES

### Example 1

Toxicity screen was done on a WST-CEM T-cell line and the minimum percent verses a no drug control was measured.

Minimum Inhibitory Concentration (MIC) Assays:

The assays described below were used to measure the minimum inhibitory concentration (MIC) of a compound necessary to completely inhibit visible growth of the organism tested. These assays are adapted from NCCLS protocols M7-A4 and M27-A (NCCLS vol 17:9 and vol 17:2) as modified by Sandven, S. Clin. Micro. (1999) 37:12, p.3856-3859. MIC values for Aspergillus fumigatus were determined using NCCLS protocol M38-P.

Inoculum Preparation, Incubation and Reading Results

All compounds were dissolved in 100% DMSO to a stock concentration of 10 mM and use fresh or stored at −80° C. Stock compounds were kept frozen until needed and used freshly with no more then one freeze-thaw cycle. When used for test purposes, compounds were diluted in the appropriate media depending on the organism being tested.

For yeast and aspergillus species, seven 1:2 serial dilutions of compound in appropriate media buffered with MOPS at pH 7.0 were prepared such that the final starting test compound concentrations were 50.0 uM for yeast and 50 uM aspergillus species. For bacteria, dilutions were made in growth media used for the particular bacteria being tested.

Yeast

Five well-separated colonies from a 24 hr Sabouraud Dextrose plate incubated at 35C were picked and resuspended into 5.0 ml of normal saline. The O.D.₅₃₀ was read and the culture was adjusted to 0.5 McFarland units with normal saline. A 1:2000 dilution was made with RPMI 1640 media buffered with MOPS at pH 7.0 and 100 μL of this inoculum preparation was added to an equal volume of test compound-containing media. 25 μL of the redox indicator Alamar Blue (Biosource International) was added to each well and the plates were incubated for 48 h at 35 C. Wells having yeast growth changed color from blue to pink. Accordingly, the MIC was calculated based on the well with the lowest concentration which did not change color from blue to pink, e.g., growth was inhibited.

Bacteria

Inoculums are made in the same manner as yeast except all dilutions are made in normal saline, with a final dilution of 1:200 and an inoculum of 10 μL. Solid and liquid media, as well as plate incubation times for the various organisms tested, are listed in Table 1 below. VRE are vancomycin resistant enterococci, BM4147 and UCD-3 represent two different sources of VRE. MRSA are methylacillin resistant Staphylococcus Aureus.

TABLE 1

| | | Solid | | |
|---|---|---|---|---|
| | Liquid | media | | |
| Organism | media | (agar) | 96 well plate incubation time | Definition |
| VRE-UCD3 | BHI | BHIA | No vancomycin - 16 h | BHI—BrainHeart Infusion |
| | | | 25 μg/mL Vancomycin - 24 h | |
| VRE-CSUC4 | BHI | BHIA | No vancomycin - 16 h | BHI—Brain Heart Infusion |
| | | | 25 μg/mL Vancomycin - 24 h | |
| VRE-UL17 | BHI | BHIA | No vancomycin - 16 h | BHI—Brain Heart Infusion |
| | | | 25 μg/mL Vancomycin - 24 h | |
| VRE-BM4147 | BHI | BHIA | No vancomycin - 16 h | BHI—Brain Heart Infusion |
| | | | 25 μg/mL Vancomycin - 24 h | |
| Moraxella | BHI | BHIA | 16 h | BHI—Brain Heart Infusion |
| catarrhalis | | | | |
| Bacillus | CAMHB | BHIA | 16 h | BHI—Brain Heart Infusion |
| cereus | | | | |
| Pseudomonas | CAMHB | BHIA | 16 h | BHI—Brain Heart Infusion |
| aeruginosa | | | | |
| Staphylococcus | CAMHB | BHIA | 16 h | CAMHB—Cation adjusted |
| aureus | | | | Muller Hinton broth |
| Haemophilus | HTM | Chocolate | 24 h | Chocolate Agar—Nutrient |
| influenzae | | Agar | agar + 5% heat lysed Sheep | |
| | | | | blood |
| Streptococcus | CAMHB + | MHA + | 24 h | LHB—Lysed Horse Blood |
| pneumoniae | 5% LHB | 5% SB | | |
| Candida | RPMI | SABDEX | 48 h | SABDEX-Sabouraud |
| albicans | | | | Dextrose Agar |

Filamentous fungi

Inoculums are made by incubating Aspergillus fumigatus for 7 days at 35 C on potato dextrose agar slants. Slants are then covered with 1.0 ml of 0.85% saline, one drop of Tween 20 is added and colonies are teased with a sterile transfer loop to create a suspension which is allowed to sit for 5 min so heavier particles can drop out. The upper suspension is separated and adjusted to an optical density of 0.09 to 0.11. The resulting suspension is diluted 1:50, which yields 2× the final inoculum needed. Micro dilution trays are prepared as with yeast and incubated for 48 h at 35C. For our purposes the MIC is defined as the lowest compound concentration at which no visible growth is observed after 48 h.

Compounds of this invention were tested in assays described above and were found to be active. Examples of compounds that exhibited antibacterial activity (MIC<45.5 μM) are shown in FIG. 5. Examples of compounds that exhibited antifungal activity (MIC<45.5 μM) are shown in FIG. 6. Examples of compounds that showed both antifungal and antibacterial activity are shown in FIGURE XX?.

Topoisomerase Inhibition Assays

Candida albicans (C. Albicans) topoisomerases I and II (cTop1 and cTop2) were isolated according to Fostel et al. (1992) and Shen et al. (1992). Human topoisomerases I and II (hTop1 and hTop2) were purchased from Topogen (Columbus, Ohio).

Inhibition of Topoisomerase I

Effects of GL compounds on DNA relaxation by topoisomerase I were studied using gel electrophoresis. Negatively supercoiled plasmid DNA (pARG, 8 kb) was used as the substrate. The reaction for C. albicans topoisomerase I was performed in 25 mM TrisHCl, pH 7.5, 50 mM NaCl, 2.5 mM MgCl₂, 0.5 mM EDTA and 50 ug/mL BSA at 35° C. The reaction was stopped at any given time by adding SDS to a final concentration of 0.5%. Subsequently, proteinase K was added to 250 ug/mL and the mixture was incubated at 60° C. for 30 min. The reaction mixture was further extracted with phenol followed by phenol:isoamyl alcohol: chloroform (25:1:24). Samples were loaded on 0.8% agarose gel and subject to electrophoresis using 1× TBE. Different DNA intercalators were used for better gel resolution. Ethidium bromide was sometimes added to both the gel and the running buffer to 0.25 ug/mL. In other cases, chloroquine was added to 0.25 ug/mL to separate the DNA topoisomers.

Inhibition of Topoisomerase II

Effects of GL compounds on topoisomerase II were investigated by monitoring decatenation reactions using entangled kinetoplast DNA (Topogen). The decatenation reaction was performed in 10 mM TrisHCl, pH 7.5, 50 mM NaCl, 50 mM KCl, 5 mM MgCl₂, 0.1 mM EDTA and 0.5 mM ATP. The reaction was stopped at any given time by adding SDS to a final concentration of 1%. Subsequently, proteinase K was added to 250 ug/mL and the mixture was incubated at 60° C. for 30 min. The reaction mixture was further extracted with phenol followed by phenol:isoamyl alcohol:chloroform (25:1:24). Samples were loaded on 0.8% agarose gel and subject to electrophoresis using 1× TBE. Ethidium bromide was added to both the gel and the running buffer to 0.25 ug/mL.

DNA Binding Properties of Compounds of this Invention

Fluorescence Studies

When compounds prefer to bind to the minor groove of dsDNA, they induce DNA duplex formation. Hybridization of complementary fluorescently labeled strands brings the two labels, fluorescein and dabcyl, in close proximity, thus quenching the fluorescence of fluorescein. Therefore, this hybridization stabilization assay (“HSA”) can be used to measure ligand binding to double-stranded DNA.

The DNA binding properties of several compounds of this invention were investigated by fluorescence spectroscopy. The 11-bp oligo CGA₈G (“FQ11”) having fluorescein at the 5′ end on one strand and dabcyl at the 3′ end on the complementary strand was used as the AT-rich ligand binding target. At room temperature, FQ11 remains largely single-stranded in the HEN buffer (10 mM HEPES, pH 7.2, 0.1 mM EDTA and 10 mM NaCl).

Fluorescence was measured at the excitation wavelength of 485 nm and the emission wavelength of 530 nm using a 96-well plate fluoreader (PE CytoFluor® Series 4000). The FQ11 concentration was kept at 5 nM (for duplex concentration) for the binding experiments and varying concentrations of ligands were added. All experiments were performed in duplicate in the HEN buffer at room temperature unless otherwise stated. Standard deviations were calculated based on the duplicate experiments. The fluorescence signal was normalized against the fluorescence in the absence of compounds. Decreasing fluorescence signals with increasing ligand concentrations indicated binding of the ligand to dsDNA. Through this least-square fitting procedure, apparent dissociation constants (K_{d,app}) for each compound tested were calculated. The studies demonstrated that compounds of this invention bind to DNA very tightly, with apparent K_{d,app} values below 100 nM for most compounds tested.

Circular Dichroism Studies

Because of the electronic interactions between ligand and DNA, ligand binding can often induce circular dichroism (“CD”) signals that are absent when DNA or ligand is alone in solution. DNA binding of compounds of this invention were determined using CD spectroscopy.

All solution conditions were the same as described above. PolydA-polydT was used at 50 μM. CD signal was monitored using a JASCO J-600 CD polarimeter at room temperature. The results showed binding properties that indicated a 2:1 complex. The dramatic CD change in the DNA absorbing region (260-300 nm) upon binding of these compounds demonstrated that compounds of this invention induced DNA conformational changes.

DNA Thermal Melting Studies

Interactions between DNA and compounds of this invention were investigated using thermal melting techniques monitored at UV wavelength 260 nm. All investigated compounds showed a stabilization effect on DNA duplex formation.

During melting experiments, 3 uM GCGA3T3CGC (A3T3) oligo duplex was mixed with 6 uM of compound in HEN buffer in a total volume of 200 uL. The UV absorbance was monitored at 260 nm with a Beckman UV spectrophotometer with temperature control. The melting temperature (Tm) where half of the duplex dissociates was determined at relative absorbance of 0.5. The free A3T3 has a Tm of approximately 42° C. With the presence of ligands, the Tm increases. The results indicated compounds of this invention tend to stabilize duplex DNA by binding to the minor groove. Increases in Tm have also been observed for duplex oligo CGATTATTAAGC in the presence of the compound.

### Anti-Tumor Assays

The anti-tumor properties of the compounds of this invention were tested according to a protocol adapted from a National Cancer Institute protocol (http://www.dtp.nci.nih.gov/branches/btb/ivclsp.html). The protocol is an enzyme based colorimetric assay using the WST-1 reagent. Three human transformed cell lines were used in this screen: NCI-H460, MCF7 and SF268, which originated from lung, breast and central nervous system tumors, respectively. The cells were maintained in RPMI media supplemented with 10% FBS and the antibiotics penicillin and streptomycin.

In a typical test, cells were resuspended in RPMI media that lacked phenol red but contained penicillin, streptomycin and only 5% FBS. Then 100 microliters of resuspended NCI-H460, MCF7 and SF268 cells were seeded at a density of 5000, 10,000 and 8500 cells per well, respectively, onto 96-well microtiter plates. This seeding density results in wells that are approximately 90% confluent at the end of three days. After plating, the cells were incubated at 37° C. in 5% CO₂ for 24 hours before compounds were added for testing for anti-tumor activity.

A 10 mM stock solution of each compound tested was prepared in DMSO. 7.5 microliters of the compound in DMSO were added to 1.5 ml. of the 5% FBS RPMI media containing penicillin and streptomycin but lacking phenol red. 100 microliters of the compound was then added to the seeded well to prepare a final compound concentration of 50 micromolar. Following the addition of compound, the cells were incubated for 48 hours. After the 48 hour incubation, WST-1 reagent (Roche Molecular Biochemicals, cat. No. 1644807) was added to determine the effect of the compound on cell proliferation. The WST-1 reagent is a tetrazolium salt, a red compound that is cleaved by mitochondrial enzymes of respiring cells to form a yellow compound, formazan. The presence of formazan is quantified spectrophotometrically at 440 nm. The amount of formazan detected is directly proportional to the number of viable cells contained within the well. If a compound inhibited cell proliferation by 80% of the no compound control, a secondary screen was performed to determine the compound concentration that inhibits proliferation by 50% (IC₅₀). The protocol for the secondary screen is exactly the same as described above, except that the amounts of compound used was varied in approximately 3-fold dilutions ranging from 50 micromolar to 40 nanomolar. The data for compounds several compounds are presented in the tables below.

All patents, patent applications and publications cited in this application are hereby incorporated by reference in their entirety for all purposes to the same extent as if each individual patent, patent application or publication were so individually denoted.

TABLE 1
Compounds of this invention were tested in the following assays and found to be
active. An active compound had a minimum inhibitory concentration (“MIC”)
value of at least 1 mM. Results of several compounds tested are shown in the table below.

| | | | | | MIC | | | | | | |
|---|---|---|---|---|---|---|---|---|---|---|---|
| | | | | | Assay | | | | | | |
| MIC | | | | | Other | | | | | | MIC |
| Assay | VRE Strains: | | | N/D not done | Strains: | | | | | | Assay |
| Com- | UCD-3 | CSUC-4 | UL-17 | BM4147 | M. | B. | P. | S. | H | S. | C. |

| pound | van₀ | van₂₅ | van₀ | van₂₅ | van₀ | van₂₅ | van₀ | van₂₅ | catarrhalis | cereus | aeruginosa | aureus | influenzae | pneumoniae | Albicans | |
|---|---|---|---|---|---|---|---|---|---|---|---|---|---|---|---|---|
| 10 | >45.5 | >45.5 | >45.5 | >45.5 | >45.5 | >45.5 | >45.5 | >45.5 | >45.5 | >45.5 | >45.5 | >45.5 | >45.5 | >45.5 | >44.4 | uM |
| 21 | >45.5 | >45.5 | >45.5 | >45.5 | >45.5 | >45.5 | >45.5 | >45.5 | >45.5 | >45.5 | >45.5 | >45.5 | >45.5 | >45.5 | >44.4 | uM |
| 42 | 45.5 | 45.5 | 22.7 | 22.7 | 22.7 | 45.5 | >45.5 | >45.5 | 11.4 | 11.4 | >45.5 | >45.5 | >45.5 | 1.4 | >44.4 | uM |
| 113 | 22.7 | 11.4 | 22.7 | 45.5 | >45.5 | 22.7 | 22.7 | 22.7 | 5.7 | 11.4 | >45.5 | >45.5 | >45.5 | 11.4 | >44.4 | uM |
| 73 | n/d | n/d | n/d | n/d | n/d | n/d | n/d | n/d | n/d | n/d | n/d | n/d | n/d | n/d | n/d | |
| 74 | 5.7 | 11.4 | 22.7 | 22.7 | 11.4 | 22.7 | 22.7 | 22.7 | 1.4 | 11.4 | >45.5 | 22.7 | >45.5 | 2.8 | >44.4 | uM |
| 103 | >45.5 | >45.5 | >45.5 | >45.5 | >45.5 | >45.5 | >45.5 | >45.5 | >45.5 | >45.5 | >45.5 | >45.5 | n/d | >45.5 | 5.6- | uM |
| | | | | | | | | | | | | | | | 11.1 | |
| 101 | >45.5 | >45.5 | >45.5 | >45.5 | >45.5 | >45.5 | >45.5 | >45.5 | >45.5 | >45.5 | >45.5 | >45.5 | >45.5 | >45.5 | 22.2- | uM |
| | | | | | | | | | | | | | | | >44.4 | |
| 102 | >45.5 | >45.5 | >45.5 | >45.5 | >45.5 | >45.5 | >45.5 | >45.5 | >45.5 | >45.5 | >45.5 | >45.5 | >45.5 | >45.5 | >44.4 | uM |
| 100 | >45.5 | >45.5 | >45.5 | >45.5 | >45.5 | >45.5 | >45.5 | >45.5 | >45.5 | >45.5 | >45.5 | >45.5 | >45.5 | >45.5 | >44.4 | uM |
| 129 | >45.5 | >45.5 | >45.5 | >45.5 | >45.5 | >45.5 | >45.5 | >45.5 | >45.5 | >45.5 | >45.5 | >45.5 | >45.5 | >45.5 | >44.4 | uM |
| 131 | >45.5 | >45.5 | >45.5 | >45.5 | >45.5 | >45.5 | >45.5 | >45.5 | >45.5 | >45.5 | >45.5 | >45.5 | >45.5 | >45.5 | >44.4 | uM |
| 133 | >45.5 | >45.5 | >45.5 | >45.5 | >45.5 | >45.5 | >45.5 | >45.5 | >45.5 | >45.5 | >45.5 | >45.5 | >45.5 | >45.5 | 22.2 | uM |
| 135 | >45.5 | >45.5 | >45.5 | >45.5 | >45.5 | >45.5 | >45.5 | >45.5 | 45.5 | 45.5 | >45.5 | 22.7 | >45.5 | >45.5 | 11.1 | uM |
| 130 | >45.5 | >45.5 | >45.5 | >45.5 | >45.5 | >45.5 | >45.5 | >45.5 | 45.5 | 45.5 | 45.5 | 45.5 | 45.5 | 45.5 | 5.6 | uM |
| 132 | >45.5 | >45.5 | >45.5 | >45.5 | >45.5 | >45.5 | >45.5 | >45.5 | 45.5 | 45.5 | 45.5 | 45.5 | 45.5 | 45.5 | 1.4 | uM |
| 134 | >45.5 | >45.5 | >45.5 | >45.5 | >45.5 | >45.5 | >45.5 | >45.5 | 45.5 | 45.5 | 45.5 | 45.5 | 45.5 | 45.5 | 5.6 | uM |
| 136 | >45.5 | >45.5 | >45.5 | >45.5 | >45.5 | >45.5 | >45.5 | >45.5 | 45.5 | 45.5 | 45.5 | 45.5 | 45.5 | 45.5 | 22.2 | uM |
| 122 | >45.5 | >45.5 | >45.5 | >45.5 | >45.5 | >45.5 | >45.5 | >45.5 | 45.5 | 22.7 | >45.5 | 22.7 | >45.5 | >45.5 | 5.6 | uM |
| 124 | >45.5 | >45.5 | >45.5 | >45.5 | >45.5 | >45.5 | >45.5 | >45.5 | 5.7 | 11.4 | >45.5 | 11.4 | >45.5 | >45.5 | 5.6 | uM |
| 126 | 11.4 | 11.4 | 22.7 | 22.7 | 45.5 | 22.7 | >45.5 | >45.5 | 2.8 | 5.7 | >45.5 | 11.4 | >45.5 | 5.7 | >44.4 | uM |
| 128 | >45.5 | >45.5 | >45.5 | >45.5 | >45.5 | >45.5 | >45.5 | >45.5 | >45.5 | >45.5 | >45.5 | >45.5 | >45.5 | >45.5 | >44.4 | uM |
| 121 | >45.5 | >45.5 | >45.5 | >45.5 | >45.5 | >45.5 | >45.5 | >45.5 | 45.5 | 45.5 | >45.5 | 45.5 | >45.5 | >45.5 | >44.4 | uM |
| 123 | >45.5 | >45.5 | >45.5 | >45.5 | >45.5 | >45.5 | >45.5 | >45.5 | 22.7 | >45.5 | >45.5 | 45.5 | >45.5 | 45.5 | >44.4 | uM |
| 125 | >45.5 | >45.5 | >45.5 | >45.5 | >45.5 | >45.5 | >45.5 | >45.5 | 11.4 | >45.5 | >45.5 | >45.5 | >45.5 | 45.5 | >44.4 | uM |
| 129 | >45.5 | >45.5 | >45.5 | >45.5 | >45.5 | >45.5 | >45.5 | >45.5 | >45.5 | >45.5 | >45.5 | >45.5 | >45.5 | >45.5 | >44.4 | uM |
| 137 | >45.5 | >45.5 | >45.5 | >45.5 | >45.5 | >45.5 | >45.5 | >45.5 | >45.5 | n/d | >45.5 | >45.5 | >45.5 | 22.7 | >44.4 | uM |
| 138 | >45.5 | >45.5 | >45.5 | >45.5 | >45.5 | >45.5 | >45.5 | >45.5 | 22.7 | 11.4 | >45.5 | 45.5 | >45.5 | 5.7 | >44.4 | uM |
| 140 | >45.5 | >45.5 | >45.5 | >45.5 | >45.5 | >45.5 | >45.5 | >45.5 | >45.5 | >45.5 | >45.5 | >45.5 | >45.5 | >45.5 | >44.4 | uM |
| 72 | n.d | >45.5 | n.d | >45.5 | n.d | >45.5 | n.d | >45.5 | >45.5 | >45.5 | >45.5 | >45.5 | >45.5 | >45.5 | >44.4 | uM |
| 70 | n.d | >45.5 | n.d | >45.5 | n.d | >45.5 | n.d | >45.5 | >45.5 | >45.5 | >45.5 | >45.5 | >45.5 | >45.5 | >44.4 | uM |
| 66 | n.d | >45.5 | n.d | >45.5 | n.d | >45.5 | n.d | >45.5 | >45.5 | >45.5 | >45.5 | >45.5 | >45.5 | >45.5 | >44.4 | uM |
| 67 | n.d | >45.5 | n.d | >45.5 | n.d | >45.5 | n.d | >45.5 | >45.5 | >45.5 | >45.5 | >45.5 | >45.5 | >45.5 | >44.4 | uM |
| 68 | n.d | >45.5 | n.d | >45.5 | n.d | >45.5 | n.d | >45.5 | >45.5 | >45.5 | >45.5 | >45.5 | >45.5 | >45.5 | >44.4 | uM |
| 69 | n.d | >45.5 | n.d | >45.5 | n.d | >45.5 | n.d | >45.5 | >45.5 | >45.5 | >45.5 | >45.5 | >45.5 | >45.5 | >44.4 | uM |
| 71 | n.d | >45.5 | n.d | >45.5 | n.d | >45.5 | n.d | >45.5 | >45.5 | >45.5 | >45.5 | >45.5 | >45.5 | >45.5 | >44.4 | uM |
| 76 | n.d | >45.5 | n.d | n.d | n.d | n.d | n.d | >45.5 | nd | >45.5 | >45.5 | >45.5 | nd | nd | >44.4 | uM |
| 77 | n.d | >45.5 | n.d | n.d | n.d | n.d | n.d | >45.5 | nd | 22.7 | >45.5 | 45.5 | nd | nd | >44.4 | uM |
| 78 | n.d | >45.5 | n.d | n.d | n.d | n.d | n.d | >45.5 | nd | 22.7 | >45.5 | 45.5 | nd | nd | | uM |
| 79 | n.d | 11.4 | n.d | n.d | n.d | n.d | n.d | 45.5 | nd | 11.4 | >45.5 | >45.5 | nd | nd | | uM |
| 80 | n.d | >45.5 | n.d | n.d | n.d | n.d | n.d | >45.5 | nd | 5.7 | >45.5 | >45.5 | nd | nd | >44.4 | uM |
| 81 | n.d | >45.5 | n.d | n.d | n.d | n.d | n.d | >45.5 | nd | 5.7 | >45.5 | 45.5 | nd | nd | 22.2 | uM |

TABLE 2
Compounds of this invention were tested in the following assays and found to be active.
An Active compound had a minimum inhibitory concentration (“MIC”) value of at least
1 mM. Results of several compounds tested are shown in the table below.

| | | | MIC | | | | | | | |
|---|---|---|---|---|---|---|---|---|---|---|
| | | | Assay | | | | | | | |
| MIC | | | Other | | | | | | MIC | |
| Assay | VRE Strains: | N/D not done | Strains: | | | | | | Assay | |
| Com- | UCD-3 | BM4147 | M | B | A | | E | | C | |
| pound | van₂₅ | van₂₅ | catarrhalis | cereus | fumigatus | MRSA | coli | CEM | Albicans | |
| 172] | >45.5 | >45.5 | | n/d | >50 | >45.5 | >45.5 | 25.5% ND | >44.4 | uM |
| 189b | 5.7 | 2.8/5.7 | | 2.8 | 3.1 | 1.4/5.7 | 45.5 | 10.2% ND | 5.6/11.1 | uM |
| 189a | 11.4 | 11.4 | | 2.8 | 12.5 | 2.8/11.4 | 11.4 | 7.7% ND | 22.2 | uM |
| 191 | 11.4 | 5.711.4 | | 5.7 | 12.5 | 2.8/5.7 | 22.7 | 3.7% ND | 11.1 | uM |
| 170 | >45.5 | >45.5 | | n/d | >50 | >45.5 | >45.5 | 90.0% ND | >44.4 | uM |
| 171 | >45.5 | >45.5 | | n/d | >50 | >45.5 | >45.5 | 91.1% ND | >44.4 | uM |
| 174 | >45.5 | >45.5 | | n/d | >50 | >45.5 | >45.5 | 92.9% ND | >44.4 | uM |
| 169 | >45.5 | >45.5 | | >45.5 | >50 | >45.5 | 45.5 | 100% ND | >44.4 | uM |
| 173 | >45.5 | >45.5 | | n/d | >50 | >45.5 | >45.5 | 87.2% ND | >44.4 | uM |
| 188b | 5.7/11.4 | 5.7/11.4 | | 22.7 | >50 | 2.8/5.7 | 22.7 | 9.2% ND | 22.2 | uM |
| 188a | 11.4 | 11.4 | | 22.7 | >50 | 11.4 | 22.7 | 6.9% ND | >44.4 | uM |

What is claimed is:

1. A compound of Formula (I): 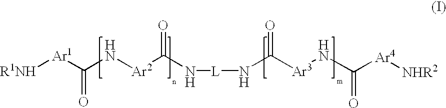
wherein:
R¹ and R² are, independently of each other:
(i) hydrogen;
(ii) alkyl; or
(iii) —COR³ wherein R³ is selected from the group consisting of alkyl, amino, monosubstituted amino, disubstituted amino, or alkyl substituted with one, two or three substituents selected from the group consisting of amino, monosubstituted amino, disubstituted amino, guanidino, amidino, aminoacyl, —NHCOR^{a} (wherein R^{a} is hydrogen, alkyl, substituted alkyl, aryl, substituted aryl, aralkyl, substituted aralkyl, cycloalkyl, substituted cycloalkyl, cycloalkylalkyl, substituted cycloalkylalkyl, heteroaryl, substituted heteroaryl, heteroaralkyl, or substituted heteroaralkyl), —NHCONHR^{a} (wherein R^{a} is as defined above), aryl, substituted aryl, heteroaryl, substituted heteroaryl, carboxy, alkoxycarbonyl, and —OR^{b} (where R^{b} is hydrogen, alkyl, substituted alkyl, aryl, substituted aryl, aralkyl, substituted aralkyl, cycloalkyl, substituted cycloalkyl, cycloalkylalkyl, substituted cycloalkylalkyl, heteroaryl, substituted heteroaryl, heteroaralkyl, or substituted heteroaralkyl), provided that at least one of R¹ and R² is a group that can form a pharmaceutically acceptable acid addition salt;
n and m are independently an integer from 0 to 4; and
Ar¹, Ar², Ar³, and Ar⁴ are independently selected from the group consisting of arylene, substituted arylene, and optionally substituted heteroarylene; and
L is:
(i) alkylene;
(ii) alkylene substituted with one, two or three substituent(s) selected from the group consisting of aryl, —CONHR⁴ (wherein R⁴ is hydrogen, alkyl, substituted alkyl, hydroxyalkyl, alkoxyalkyl, aminoalkyl, aryl, substituted aryl, aralkyl, substituted aralkyl, heteroaryl, substituted heteroaryl, heteroaralkyl, or substituted heteroaralkyl, heterocyclic, substituted heterocyclic, heterocyclicalkyl, heteroarylthioalkyl, or —(CHR⁵)ₙ₁—CO—(NH—Ar³—CO)ₘ—NH—Ar⁴—CO—NHR³ where n1 is 1 to 3, R⁵ is hydrogen or alkyl, substituted alkyl, and Ar³, m, Ar⁴, and R³ are as defined above), —CONHNHR⁶ [wherein R⁶ is alkyl, substituted alkyl, aryl, substituted aryl, aralkyl, substituted aralkyl, —COR⁷, —COOR⁸ (wherein R⁷ and R⁸ are independently of each other alkyl, substituted alkyl, aryl, substituted aryl, aralkyl, cycloalkyl, substituted cycloalkyl, cycloalkylalkyl, substituted cycloalkylalkyl, heteroaryl, substituted heteroaryl, or heteroaralkyl), heteroaryl, or heteroaralkyl], —NHR⁹ (wherein R⁹ is hydrogen, alkyl, substituted alkyl, hydroxyalkyl, alkoxyalkyl, aminoalkyl, aminoalkylcarbonyl, or heterocycliccarbonyl), and guanidino; or
(iii) -(alkylene)ₓ-Z-(alkylene)_{y}-(Z^{a})_{z}- wherein x, y and z are independently 0, 1, or 2 and Z and Z^{a} are, independently of each other, phenylene, cycloalkylene optionally fused to one or two phenylene ring(s), heterocyclene, —O—, —S—, —NR¹⁰— [wherein R¹⁰ is hydrogen, alkyl, substituted alkyl, cycloalkylcarbonyl, hydroxyalkyl, alkoxyalkyl, aminoalkyl, —CONHR⁴, —COR⁷, —COOR⁸ (where R⁴, R⁷ and R⁸ are as defined above), —SO₂R¹¹ (where R¹¹ is alkyl, substituted alkyl, aryl, substituted aryl, aralkyl, substituted aralkyl, heteroaryl, substituted heteroaryl, heteroaralkyl, or substituted heteroaralkyl) or —(CHR⁵)ₙ₂—NH—(CO—Ar³—NH)ₘ—CO—Ar⁴—NHR² where n2 is 2 to 4, R⁵ is hydrogen, alkyl, or substituted alkyl, and Ar³, m, Ar⁴, and R² are as defined above], —CO—NH—, or —NH—CO—, provided that when Z and/or Z^{a} is —NR¹⁰— then it is separated from another nitrogen atom by at least two carbon atoms;
or a pharmaceutically acceptable salt thereof.

2. The compound of claim 1 wherein Ar¹, Ar² Ar³ and Ar⁴ are independently an optionally substituted heteroarylene.

3. The compound of claim 2 wherein Ar¹, Ar², Ar³ and Ar⁴ are independently a 1-methylpyrrole that is linked to the carbonyl group at the 2-position and the amino group at the 4-position of the pyrrole ring.

4. The compound of claim 1 wherein n and m are 0 or 1.

5. The compound of claim 4 wherein Ar¹, Ar², Ar³ and Ar⁴ are independently an optionally substituted heteroarylene.

6. The compound of claim 1 wherein R¹ and R² are independently —COR³.

7. The compound of claim 6 wherein R¹ and R² are independently aminomethylcarbonyl, 1-amino-4-guanidinobutylcarbonyl, 1,4-diaminobutylcarbonyl, 1,5-diaminopentyl-carbonyl, 1-amino-5-(3,4-difluorophenylureido)pentylcarbonyl, 1-(3,4-difluoro-phenylureido)-4-guanidinobutylcarbonyl, 1-[4-(N,N-(2-chloroethyl)-aminophenyl-butanoyl)]amino-4-guanidinobutylcarbonyl, 1-amino-5-[4-(N,N-(2-chloroethyl)-aminophenyl-butanoyl)]aminopentylcarbonyl, or pyrene-1-ylmethyloxy.

8. The compound of claim 1 wherein L is alkylene.

9. The compound of claim 8 wherein L is 1,2-ethylene, 1,3-propylene, 1,4-butylene, 1,6-hexylene, 1,8-octylene, 1,12-dodecylene, 1-methylethylene, or 1,2-hexadecylene.

10. The compound of claim 1 wherein L is substituted alkylene.

11. The compound of claim 10 wherein L is meso-1,2-diphenylethylene, 1-(p-nitrophenylaminocarbonyl)-1,5-pentylene, 1-(napth-2-ylaminocarbonyl)-1,5-pentylene, 1-(pentafluorophenylhydrazidocarbonyl)-1,5-pentylene, 1-(5-trifluoro-pyrimidin-2-ylhydrazidocarbonyl)-1,5-pentylene, 1-(2-pyrene-1-ylethylamino-carbonyl)-1,5-pentylene, 1-[2-(6-nitrobenzimidazol-1-ylethylaminocarbonyl]-1,5-pentylene, 1-[2-(indol-3-yl)-ethylaminocarbonyl]-1,5-pentylene, 1-[2-(5-fluoroindol-3-yl)ethylaminocarbonyl]-1,5-pentylene, 1-[2-(4-nitrophenyl)ethylaminocarbonyl]-1,5-pentylene, 1-(benzyloxycarbonyl-hydrazidocarbonyl)-1,2-ethylene, 1-(napth-1-ylaminocarbonyl)-1,5-pentylene, 1-(4-pyrene-1-ylbutylaminocarbonyl)-1,5-pentylene, 1-(2-(2-trifluoromethylquinolin-4-yl)thio-ethylaminocarbonyl)-1,5-pentylene, 1-(pentafluorophenylhydrazidocarbonyl)-1,4-butylene, 1-(4-pyrene-1-ylmethylaminocarbonyl)-1,5-pentylene, 1-(2-hydroxyethylaminocarbonyl)-1,5-pentylene, 1-(2-aminoethylaminocarbonyl)-1,5-pentylene, 1-(3-dimethylaminopropyl-aminocarbonyl)-1,5-pentylene, 1-(bis-(2-aminoethyl)aminoethylaminocarbonyl)-1,5-pentylene, 1-(N-(2-aminoethyl)aminoethylaminocarbonyl)-1,5-pentylene, 2-(amino-methylcarbonyl-amino)-1,3-propylene, or 2-(3-hydroxypyrrolidin-5-ylcarbonyl-amino)-1,3-propylene.

12. The compound of claim 1 wherein L is -(alkylene)ₓ-Z-(alkylene)_{y}-(Z^{a})_{z}-.

13. The compound of claim 12 wherein L is m-xylene, p-xylene, 2,7-fluorendiyl, bis-(3-N-benzyloxycarbonylamino)propylene [—(CH₂)₃—N(BzOCO—)—(CH₂)₃—], bis-(2-napth-2-ylsulfonylamino)ethylene [—(CH₂)₂—N(—SO₂napth-2-yl)-(CH₂)₂—], bis-(2-N-3,5-dinitrophenylcarbonylamino)ethylene [—(CH₂)₂—N(—CO-3,5-dinitrophenyl)-(CH₂)₂—], 1,3-cyclohexyl-bis-methylene [—(CH₂)-(1,3-C₆H₁₀)—(CH₂)—], 1,4-cyclohexyl-bis-methylene [—(CH₂)-(1,4-C₆H₁₀)—(CH₂)—], 4,4′-methylene-bis-1,4-cyclohexylene [-(1,4-C₆H₁₀)—(CH₂)-(1,4-C₆H₁₀)—], 1,2-cyclohexylene (1,2-C₆H₁₀—), bis-(2-adamantyl1-ylcarbonylamino)ethylene, bis-(3-N-methylamino)propylene [—(CH₂)₃—N(—CH₃)—(CH₂)₃—], bis-(3-amino)propylene [—(CH₂)₃—NH—(CH₂)₃—], 1,4-piperazino-bis-propylene [—(CH₂)₃-(1,4-piperazino)-(CH₂)₃—], bis-(2-(2-aminoethyl)amino)ethylene [—(CH₂)₂—N(—(CH₂)₂NH₂)—(CH₂)₂—], and bis-(2-amino)ethylene [—(CH₂)₂—NH—(CH₂)₂—].

14. A pharmaceutical composition comprising a therapeutically effective amount of a compound of claims 1-13 and a pharmaceutically suitable carrier.

15. A method for the treatment of diseases caused by pathogenic organisms, which comprises administering to a mammal in need of such treatment a therapeutically effective amount of a pharmaceutical composition containing a therapeutically effective amount of a compound of claims 1-13 and a pharmaceutically suitable carrier.

16. The method of claim 15 wherein the disease is cancer.

17. A compound of the formula (II). 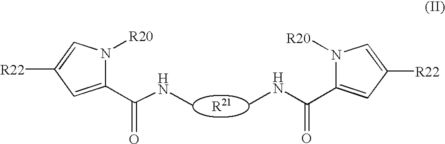
wherein R²¹ is an arylene, heteroarylene, substituted arylene or substituted heteroarylene; each R²⁰ is independently alkyl or substituted alkyl; and each R²² is independently guanidino or amidino.

18. The compound of claim 17 where in R²¹ is selected from the group consisting of 1,4-phenylene, 1,3-phenylene, 1,3-phenylene, 1,4-pyridylene, 1,3-pyridylene, 2,4-pyrimidinylene, 2,5-pyrimidinylene, 3,5-(1,2,4-)trizolene, 2,5-thiazolene, and 2,7-naphthylene; wherein said 1,4-phenylene and 1,3-phenylene are optionally substituted; and each R²⁰ is independently selected from the group consisting of methyl, ethyl, propyl, isoamyl, and cyclopropylmethyl.

19. The compound of claim 18 selected from the group consisting of 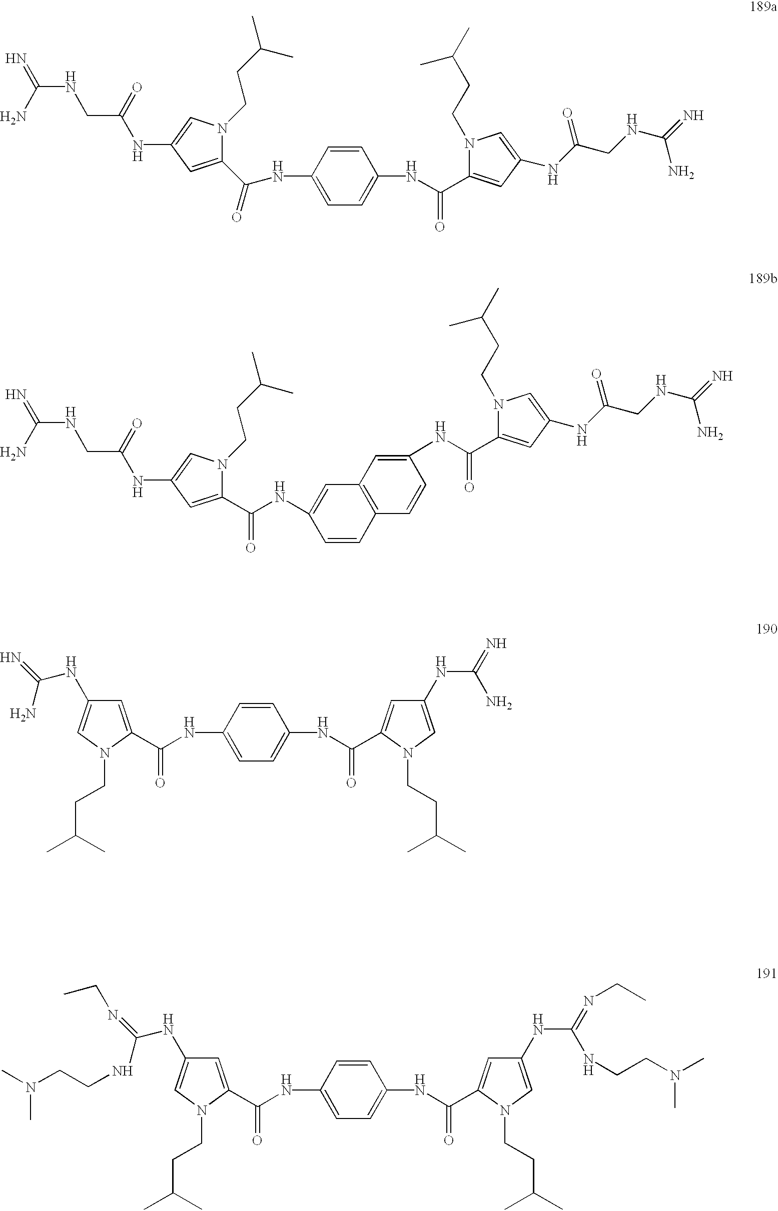
and pharmaceutically acceptable salts thereof.

20. A compound of the formula (VII) 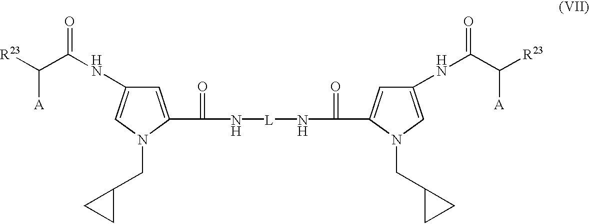
Wherein
L is selected from the group consisting of alkylene and cycloalkylene;
A is an amino acid side chain; and
R²³ is selected from the group consisting of guanidino, amino, and ornithylamino.

21. A compound of claim 20 selected from the group consisting of 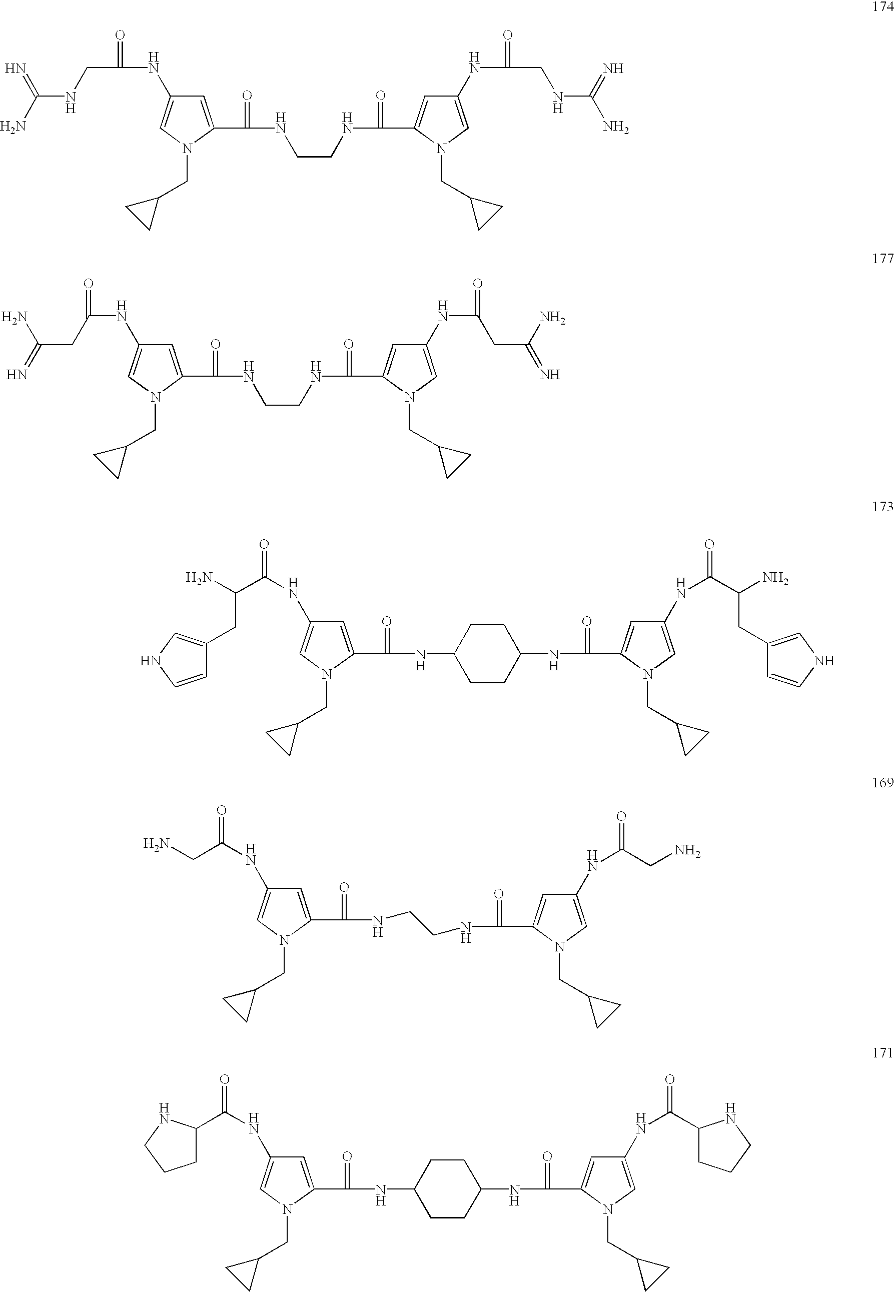
and pharmaceutically acceptable salts thereof.

22. A compound selected from the group consisting of: Compounds 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 48, 49, 50, 51, 52, 53, 54, 55, 56, 57, 58, 63, 66, 67, 68, 69, 70, 71, 72, 73, 74, 76, 77, 78, 79, 80, 81, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158. (depicted on pages 17-25, and FIGS. 1-8).
